SEQUENCE LISTING (5 sequences)

SEQ ID NO: 1 - protein, 631 aa, Escherichia virus N15
MSKVKIGELINTLVNEVEAIDASDRPQGDKTKRIKAAAARYKNALFNDKRKFRGKGLQKRITANTFNAYMSRARKRFDDKLHHSFDKNINKLSEKYPLYSEELSSWLSMPTANIRQHMSSLQSKLKEIMPLAEELSNVRIGSKGSDAKIARLIKKYPDWSFALSDLNSDDWKERRDYLYKLFQQGSALLEELHQLKVNHEVLYHLQLSPAERTSIQQRWADVLREKKRNVVVIDYPTYMQSIYDILNNPATLFSLNTRSGMAPLAFALAAVSGRRMIEIMFQGEFAVSGKYTVNFSGQAKKRSEDKSVTRTIYTLCEAKLFVELLTELRSCSAASDFDEVVKGYGKDDTRSENGRINAILAKAFNPWVKSFFGDDRRVYKDSRAIYARIAYEMFFRVDPRWKNVDEDVFFMEILGHDDENTQLHYKQFKLANFSRTWRPEVGDENTRLVALQKLDDEMPGFARGDAGVRLHETVKQLVEQDPSAKITNSTLRAFKFSPTMISRYLEFAADALGQFVGENGQWQLKIETPAIVLPDEESVETIDEPDDESQDDELDEDEIELDEGGGDEPTEEEGPEEHQPTALKPVFKPAKNNGDGTYKIEFEYDGKHYAWSGPADSPMAAMRSAWETYYS

SEQ ID NO: 2 - DNA, 56 bp, Unknown
  Description of Unknown Enzyme recognition nucleic acid sequence
tatcagcacacaatagtccattatacgcgcgtataatgggcaattgtgtgctgata

SEQ ID NO: 3 - DNA, 56 bp, Unknown
  Description of Unknown Enzyme recognition nucleic acid sequence
tatcagcacacaattgcccattatacgcgcgtataatggactattgtgtgctgata

SEQ ID NO: 4 - DNA, 52 bp, Artificial Sequence
  Description of Artificial Sequence Synthetic oligonucleotide
tatcagcacacaatagtccattatacgtataatggactattgtgtgctgata

SEQ ID NO: 5 - DNA, 60 bp, Artificial Sequence
  Description of Artificial Sequence Synthetic oligonucleotide
tatcagcacacaattgcccattatacgcgcgcgcgtataatgggcaattgtgtgctgata

(19) United States
(12) Patent Application Publication
(10) Pub. No.: US 2023/0235392 A1
(43) Pub. Date: Jul. 27, 2023

(54) METHODS FOR PAIRED-END SEQUENCING LIBRARY PREPARATION

(71) Applicant: Element Biosciences, Inc., San Diego, CA (US)

(72) Inventors: Sinan ARSLAN, San Diego, CA (US); Junhua ZHAO, San Diego, CA (US); Molly HE, San Diego, CA (US); William LIGHT, San Diego, CA (US); Matthew KELLINGER, San Diego, CA (US); Michael PREVITE, San Diego, CA (US)

(21) Appl. No.: 17/999,023

(22) PCT Filed: May 19, 2021
(86) PCT No.: PCT/US21/33191

§ 371 Date: Nov. 16, 2022

Related U.S. Application Data
(60) Provisional application No. 63/027,891, filed on May 20, 2020.

(51) Int. Cl. C12Q 1/6874; C12Q 1/6855; C12Q 1/6876; C12Q 1/6806

(52) U.S. Cl. CPC C12Q 1/6874; C12Q 1/6855; C12Q 1/6876; C12Q 1/6806; C12Q 2600/16

(57) ABSTRACT

Provided herein are methods for generating circular nucleic acid molecules and circular nucleic acid libraries. The methods can be used to generate clonal populations of target nucleic acid molecules for downstream applications such as sequencing. Nucleic acid sequence methods, systems and kits are also provided for sequencing circular nucleic acid molecules.

### CROSS-REFERENCE

This application claims the benefit of U.S. Provisional Application No. 63/027,891, filed May 20, 2020, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on May 19, 2021, is named 52933-730_601_SL.txt and is 6,628 bytes in size.

### BACKGROUND

Next-generation sequencing (NGS) has provided rapidly increasing amounts of genetic information over the last two decades, which has had major implications for research and clinical practice.

### SUMMARY

Provided herein are methods for generating circular nucleic acid molecules and circular nucleic acid libraries for next-generation sequencing.

Aspects disclosed herein, in some ways, provide methods of nucleic acid sequencing, said method comprising: (a) bringing a nucleic acid sequence into contact with a surface under conditions sufficient to couple said nucleic acid sequence derivative to said surface; (b) enzymatically circularizing said nucleic acid sequence or derivative thereof to produce a circular nucleic acid sequence; (c) contacting said circular nucleic acid sequence or derivative thereof with a primer sequence complementary thereto, thereby producing a primed nucleic acid sequence; and (d) performing a nucleotide binding reaction with said primed nucleic acid sequence or derivative thereof to identify a nucleotide of said primed nucleic acid sequence or derivative thereof, which nucleotide binding reaction is performed in absence of incorporation of a nucleotide into said primed nucleic acid sequence or derivative thereof. In some embodiments, said enzymatically circularizing said nucleic acid sequence or comprises performing splint ligation. In some embodiments, (a) comprises bringing a fluid comprising said nucleic acid sequence at a concentration of less than or equal to about 1 nanomolar (nM) into contact with said surface. In some embodiments, (a) comprises bringing a fluid comprising said nucleic acid sequence at a concentration of less than or equal to about 100 picomolar (pM) into contact with said surface. In some embodiments, (a) comprises bringing a fluid comprising said nucleic acid sequence at a concentration comprising greater than or equal to about 80 picomolar (pM) into contact with said surface. In some embodiments, (a) comprises bringing a fluid comprising said nucleic acid sequence or derivative thereof at a concentration comprising between about 20 pM and about 1 nM. In some embodiments, said primed nucleic acid sequence or derivative thereof is coupled to said surface at a surface density of greater than or equal to about 4,000 primed nucleic acid sequences per micrometer (μm)². In some embodiments, said primed nucleic acid sequence or derivative thereof is coupled to said surface at a surface density of less than or equal to about 15,000 primed nucleic acid sequences per μm². In some embodiments, a plurality of colonies comprising said primed nucleic acid sequence or derivative thereof is present at said surface at a density of greater than or equal to about 300 thousand (K)/mm². In some embodiments, said colony density comprises less than or equal to about 500 K/mm². In some embodiments, said primed nucleic acid sequence or derivative thereof comprises one or more adaptors comprising an index site having a sequence complementary to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said surface comprises a hydrophilic polymer layer coupled thereto. In some embodiments, said primed nucleic acid sequence or derivative thereof comprises a concatemer of two or more repeats of an identical sequence. Some embodiments further comprise amplifying said circular nucleic acid sequence or derivative thereof using rolling circle amplification (RCA) prior to (c). Some embodiments further comprise (e) performing a primer extension reaction on said primed nucleic acid sequence or derivative thereof; and (f) repeating (a) to (e) for each successive nucleotide to identify a sequence of said primed nucleic acid sequence or derivative thereof. In some embodiments, (a)-(f) are performed in less than or equal to about 120 minutes. In some embodiments, (d)-(e) are performed in less than or equal to about 15 minutes. In some embodiments, (f) is performed in less than or equal to about 15 minutes. In some embodiments, performing said nucleotide binding reaction in (d) comprises: (i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and (ii) detecting said stable multivalent binding complex to determine said identity of said nucleotide of said primed nucleic acid sequence or derivative thereof. In some embodiments, said one or more polymer-nucleotide conjugates comprises a polymer core and a detectable label coupled thereto. In some embodiments, said one or more polymer-nucleotide conjugates comprises two or more types of said one or more polymer-nucleotide conjugates. In some embodiments, said one or more polymer-nucleotide conjugates comprises three or more types of said one or more polymer-nucleotide conjugates. In some embodiments, said one or more polymer-nucleotide conjugates comprises four types of said one or more polymer-nucleotide conjugates. In some embodiments, said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type. In some embodiments, said one or more polymer-nucleotide conjugates comprises a plurality of types of polymer-nucleotide conjugates, and wherein each of said plurality of types of said polymer-nucleotide conjugates comprises a distinct detectable label. In some embodiments, said enzymatically circularizing said nucleic acid sequence or derivative thereof in (b) comprises: (i) hybridizing a 5′ end of a single-stranded nucleic acid molecule to a 3′ end of said nucleic acid sequence or derivative thereof and hybridizing a 3′ end of said single-stranded nucleic acid molecule to a 5′ end of said nucleic acid sequence or derivative thereof, or (ii) hybridizing a 3′ end of a single-stranded nucleic acid molecule to a 5′ end of said nucleic acid sequence or derivative thereof and hybridizing a 5′ end of said single-stranded nucleic acid molecule to a 3′ end of said nucleic acid sequence or derivative thereof. In some embodiments, said single-stranded nucleic acid molecule comprises between about 20-30 contiguous nucleotides. In some embodiments, said nucleic acid sequence or derivative thereof comprises one or more unique molecular identifiers (UMI) at a 5′ end or a 3′ end thereof. Some embodiments further comprise adding one or more adaptors to a 5′ end or a 3′ end of said nucleic acid sequence or derivative thereof comprising an index site having a nucleic acid sequence corresponding to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said enzymatically circularizing said nucleic acid sequence or derivative thereof comprises ligating a 5′ end and a 3′ end of said nucleic acid sequence or derivative thereof together under conditions sufficient to produce said circular nucleic acid sequence or derivative thereof. Some embodiments further comprise performing (a) to (d) for a plurality of said nucleic acid sequence or derivative thereof. Some embodiments further comprise incorporating a nucleotide into said primed nucleic acid sequence.

Aspects disclosed herein, in some ways, provide methods of nucleic acid sequencing, said method comprising: (a) circularizing a nucleic acid sequence to provide a circular nucleic acid sequence coupled to a surface; (b) contacting said circular nucleic acid sequence or derivative thereof with a primer sequence complementary thereto, thereby producing a primed nucleic acid sequence; and (c) performing a nucleotide binding reaction with said primed nucleic acid sequence or derivative thereof to identify a nucleotide of said primed nucleic acid sequence or derivative thereof, which nucleotide binding reaction is performed in absence of incorporation of a nucleotide into said primed nucleic acid sequence or derivative thereof. In some embodiments, said circularizing said nucleic acid sequence thereof comprises performing splint ligation. In some embodiments, said circular nucleic acid sequence is coupled to said surface at a surface density of greater than or equal to about 4,000 primed nucleic acid sequences per micrometer (μm)². In some embodiments, said circular nucleic acid sequence is coupled to said surface at a surface density of less than or equal to about 15,000 primed nucleic acid sequences per μm². In some embodiments, a plurality of colonies comprising said circular nucleic acid sequence or a derivative thereof is present at said surface at a density of greater than or equal to about 300 thousand(K)/mm². In some embodiments, said colony density comprises less than or equal to about 500 K/mm². In some embodiments, said circular nucleic acid sequence or derivative thereof comprises one or more adaptors comprising an index site having a sequence complementary to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said surface comprises a hydrophilic polymer layer coupled thereto. In some embodiments, said circular nucleic acid sequence or derivative thereof comprises a concatemer of two or more repeats of an identical sequence. Some embodiments further comprise amplifying said circular nucleic acid sequence or derivative thereof using rolling circle amplification (RCA) prior to (c). In some embodiments, said rolling circle amplification is performed in at least about 10 minutes to at least about 90 minutes. Some embodiments further comprise (e) performing a primer extension reaction on said primed nucleic acid sequence or derivative thereof; and (f) repeating (a) to (e) for each successive nucleotide to identify a sequence of said primed nucleic acid sequence or derivative thereof. In some embodiments, (a)-(f) are performed in less than or equal to about 120 minutes. In some embodiments, (d)-(e) are performed in less than or equal to about 15 minutes. In some embodiments, (f) is performed in less than or equal to about 15 minutes. In some embodiments, performing said nucleotide binding reaction in (d) comprises: (i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and (ii) detecting said stable multivalent binding complex to determine said identity of said nucleotide of said primed nucleic acid sequence or derivative thereof. In some embodiments, said one or more polymer-nucleotide conjugates comprises a polymer core and a detectable label coupled thereto. In some embodiments, said one or more polymer-nucleotide conjugates comprises two or more types of said one or more polymer-nucleotide conjugates. In some embodiments, said one or more polymer-nucleotide conjugates comprises three or more types of said one or more polymer-nucleotide conjugates. In some embodiments, said one or more polymer-nucleotide conjugates comprises four types of said one or more polymer-nucleotide conjugates. In some embodiments, said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type. In some embodiments, said one or more polymer-nucleotide conjugates comprises a plurality of types of polymer-nucleotide conjugates, and wherein each of said plurality of types of said polymer-nucleotide conjugates comprises a distinct detectable label. In some embodiments, said circularizing said nucleic acid sequence or derivative thereof in (b) comprises: (i) hybridizing a 5′ end of a single-stranded nucleic acid molecule to a 3′ end of said nucleic acid sequence or derivative thereof and hybridizing a 3′ end of said single-stranded nucleic acid molecule to a 5′ end of said nucleic acid sequence or derivative thereof, or (ii) hybridizing a 3′ end of a single-stranded nucleic acid molecule to a 5′ end of said nucleic acid sequence or derivative thereof and hybridizing a 5′ end of said single-stranded nucleic acid molecule to a 3′ end of said nucleic acid sequence or derivative thereof. In some embodiments, said single-stranded nucleic acid molecule comprises between about 20-30 contiguous nucleotides. In some embodiments, said nucleic acid sequence or derivative thereof comprises one or more unique molecular identifiers (UMI) at a 5′ end or a 3′ end thereof. Some embodiments further comprise adding one or more adaptors to a 5′ end or a 3′ end of said nucleic acid sequence or a derivative thereof comprising an index site having a nucleic acid sequence corresponding to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said enzymatically circularizing said nucleic acid sequence or derivative thereof comprises ligating a 5′ end and a 3′ end of said nucleic acid sequence or derivative thereof together under conditions sufficient to produce said circular nucleic acid sequence or derivative thereof. Some embodiments further comprise performing (a) to (d) for a plurality of said nucleic acid sequence or derivative thereof. Some embodiments further comprise incorporating a nucleotide into said primed nucleic acid sequence.

Aspects disclosed herein, in some ways, provide systems of nucleic acid sequencing, said system comprising: a surface; and one or more computer processors individually or collectively programmed to implement a method comprising: (a) bringing a nucleic acid sequence into contact with said surface under conditions sufficient to couple said nucleic acid sequence or derivative thereof to said surface; (b) enzymatically circularizing said nucleic acid sequence or a derivative thereof to produce a circular nucleic acid sequence; (c) contacting said circular nucleic acid sequence or derivative thereof with a primer sequence complementary thereto, thereby producing a primed nucleic acid sequence; and (d) performing a nucleotide binding reaction with said primed nucleic acid sequence or a derivative thereof to identify a nucleotide of said primed nucleic acid sequence or derivative thereof. Some embodiments further comprise: a first fluid comprising a synthetic ligating enzyme or enzymatically-active fragment thereof, and a synthetic splint nucleic acid molecule. Some embodiments further comprise a second fluid comprising one or more nucleotide moieties and a polymerizing enzyme. In some embodiments, said surface comprises a hydrophilic polymer layer coupled thereto. Some embodiments further comprise an imaging module comprising one or more light sources, one or more optical components, and one or more image sensors operably connected to said surface for detecting said binding complex. Some embodiments further comprise a fluidics module configured to bring said nucleic acid sequence or derivative thereof into contact with said surface in (b). In some embodiments, said method further comprises: (e) performing a primer extension reaction on said primed nucleic acid sequence or derivative thereof; and (f) repeating (a) to (e) for each successive nucleotide to identify a sequence of said primed nucleic acid sequence or derivative thereof. In some embodiments, said method is performed in less than or equal to about 30 minutes. In some embodiments, said method further comprises: amplifying said circular nucleic acid sequence or a derivative thereof using rolling circle amplification (RCA) prior to (c). In some embodiments, said rolling circle amplification is performed in at least about 10 minutes to at least about 90 minutes. In some embodiments, said surface comprises an interior surface of a flow cell. In some embodiments, said concentration comprises less than or equal to about 100 picomolar (pM). In some embodiments, said concentration comprises less than or equal to about 80 picomolar (pM). In some embodiments, said concentration comprises between about 20 pM and about 1 nM. In some embodiments, said primed nucleic acid sequence or said derivative thereof is coupled said surface at a surface density of greater than or equal to about 4,000 primed nucleic acid sequences per micrometer (μm)². In some embodiments, said surface density comprises less than or equal to about 15,000 primed nucleic acid sequences per μm². In some embodiments, a plurality of colonies comprising said primed nucleic acid sequence or said derivative thereof is present at said surface with a colony density of greater than or equal to about 300 thousand (K)/mm². In some embodiments, said colony density comprises less than or equal to about 500 K/mm². In some embodiment, said primed nucleic acid sequence or said derivative thereof comprises one or more adaptors comprising an index site having a sequence complementary to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said surface comprises a hydrophilic polymer layer. In some embodiments, said hydrophilic polymer layer comprises a polymer selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. In some embodiments, said primed nucleic acid sequence or derivative thereof comprises a concatemer of two or more repeats of an identical sequence. In some embodiments, methods further comprise: (e) amplifying said circular nucleic acid sequence using rolling circle amplification (RCA). In some embodiments, methods further comprise: (e) performing a primer extension reaction of said primed nucleic acid sequence or said derivative thereof; and (f) repeating (a) to (e) for each successive nucleotide to identify a sequence of said primed nucleic acid sequence or said derivative thereof. In some embodiments, (a) to (f) are performed in less than or equal to about 30 minutes. In some embodiments, performing said nucleotide binding reaction in (d) comprises: (i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugate and a nucleotide of said primed nucleic acid sequence or derivative thereof; and (ii) detecting said stable multivalent binding complex to determine said identity of said nucleotide of primed nucleic acid sequence or derivative thereof. In some embodiments, said one or more polymer-nucleotide conjugates comprises a polymer core and a detectable label coupled thereto. In some embodiments, (d) is performed under conditions sufficient to prevent incorporation of said nucleotide moiety of said one or more polymer-nucleotide conjugates into said primed nucleic acid sequence or derivative thereof. In some embodiments, said one or more polymer-nucleotide conjugates comprises two or more types of said one or more polymer-nucleotide conjugates. In some embodiments, said one or more polymer-nucleotide conjugates comprises three or more types of said one or more polymer-nucleotide conjugates. In some embodiments, said one or more polymer-nucleotide conjugates comprises four types of said one or more polymer-nucleotide conjugates. In some embodiments, each of said types of said one or more polymer-nucleotide conjugates comprises a nucleotide moiety with a distinct nucleobase type. In some embodiments, each of said types of said one or more polymer-nucleotide conjugates comprises a distinct detectable label. In some embodiments, said enzymatically circularizing said nucleic acid sequence in (a) comprises: (i) hybridizing a 5′ end of a single-stranded nucleic acid molecule to a 3′ end of said nucleic acid sequence or derivative thereof; and (ii) hybridizing a 3′ end of said single-stranded nucleic acid molecule to a 5′ end of said nucleic acid sequence or derivative thereof. In some embodiments, said single-stranded nucleic acid molecule comprises between about 20-30 contiguous nucleotides. In some embodiments, said nucleic acid sequence or derivative thereof comprises one or more unique molecular identifiers (UMI) at a 5′ end or a 3′ end thereof. In some embodiments, methods further comprise: adding one or more adaptors to a 5′ end or a 3′ end of said nucleic acid sequence or derivative thereof comprising an index site having a nucleic acid sequence corresponding to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides. In some embodiments, said enzymatically circularizing said nucleic acid sequence or derivative thereof comprises: (i) ligating a 5′ end and a 3′ end of said nucleic acid sequence or derivative thereof under conditions sufficient to produce said circular nucleic acid sequence or said derivative thereof. In some embodiments, said methods further comprise performing (a) to (d) for a plurality of said nucleic acid sequence or derivative thereof. In some embodiments, performing said nucleotide binding reaction in (d) comprises: (i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and (ii) detecting said stable multivalent binding complex to determine said identity of said nucleotide of said primed nucleic acid sequence or derivative thereof. Some embodiments further comprise said one or more polymer-nucleotide conjugates. Some embodiments further comprise two or more types of said one or more polymer-nucleotide conjugates. Some embodiments further comprise three or more types of said one or more polymer-nucleotide conjugates. Some embodiments further comprise four types of said one or more polymer-nucleotide conjugates. In some embodiments, said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type. In some embodiments, said one or more polymer-nucleotide conjugates comprises a plurality of types of polymer-nucleotide conjugates, and wherein each of said plurality of types of said polymer-nucleotide conjugates comprises a distinct detectable label. In some embodiments, said polymer-nucleotide composition comprises a detectable label. In some embodiments, said detectable label comprises a fluorescent label. Some embodiments further comprise said nucleic acid sequence or derivative thereof, wherein said nucleic acid sequence or derivative thereof comprises one or more unique molecular identifiers (UMI) at a 5′ end or a 3′ end thereof. Some embodiments further comprise said nucleic acid sequence or derivative thereof, wherein said nucleic acid sequence or derivative thereof comprises one or more adaptors comprising an index site having a nucleic acid sequence corresponding to at least a portion of a capture nucleic acid molecule coupled to said surface. In some embodiments, said index site comprises less than or equal to about 25 contiguous nucleotides. In some embodiments, said index site comprises less than or equal to about 10 contiguous nucleotides. In some embodiments, said index site comprises between about 5 and 25 contiguous nucleotides.

Aspects disclosed herein provide kits comprising: (a) a synthetic ligating enzyme or enzymatically-active fragment thereof; and (b) a synthetic splint oligonucleotide molecule. In some embodiments, said kits further comprise instructions for: (i) preparing a nucleic acid sequencing library of a circular nucleic acid by coupling said synthetic splint oligonucleotide molecule to a linear nucleic acid fragment, and ligating a 3′ end and a 5′ end of said circular nucleic acid together using said synthetic ligating enzyme. In some embodiments, said kits further comprise instructions for bringing said circular nucleic acid into contact with a primer sequence complementary to a portion thereof under conditions sufficient to produce a primed nucleic acid template. In some embodiments, said kits further comprise instructions for sequencing said primed nucleic acid template or a derivative thereof in a nucleotide binding reaction. In some embodiments, said instructions for sequencing said primed nucleic acid template or derivative thereof in said nucleotide binding reaction comprises instructions for bringing a polymer-nucleotide conjugate comprising a polymer core and a plurality of nucleotide moieties attached thereto into contact with said primed nucleic acid template or a derivative thereof, and detecting binding of a nucleotide moiety of said plurality of nucleotide moieties and a nucleotide in said primed nucleic acid template or a derivative thereof. In some embodiments, the kits further comprise: (d) a synthetic exonuclease enzyme or enzymatically-active fragment thereof. In some embodiments, said synthetic exonuclease enzyme or enzymatically-active fragment thereof comprises a double-stranded DNA specific exonuclease. In some embodiments, said synthetic exonuclease enzyme or enzymatically-active fragment thereof comprises: (i) Lambda exonuclease; (ii) Exonuclease I; (iii) DNase1; (iv) Exonuclease V; (v) T7 exonuclease; (vi) a variant of any one of (i) to (v); or (vii) a combination of any one of (i) to (vi). In some embodiments, the kits further comprise one or more buffers comprising an elution buffer, a ligation buffer, or a combination thereof. In some embodiments, said synthetic ligating enzyme or enzymatically-active fragment thereof is a DNA ligase comprising: (i) T4 ligase; (ii) T7 ligase; (iii) T3 ligase; (iv) E. coli ligase; (v) a variant of any one of (i) to (iv); or (vi) a combination of (i) to (v). In some embodiments, said synthetic ligating enzyme or enzymatically-active fragment thereof is a thermostable ligase. In some embodiments, said synthetic ligating enzyme or enzymatically-active fragment thereof comprises an ATP-dependent double stranded DNA specific ligase. In some embodiments, said synthetic ligating enzyme or enzymatically-active fragment thereof is derived from a bacteriophage selected from the group consisting of T7, T3, and T4.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure herein are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A depicts, in accordance with some embodiments herein, a method for processing a nucleic acid molecule.

FIG. 1B depicts, in accordance with some embodiments herein, a method for processing a nucleic acid molecule.

FIG. 1C depicts, in accordance with some embodiments herein, a method for processing a nucleic acid molecule.

FIG. 1D depicts, in accordance with some embodiments herein, a method for processing a nucleic acid molecule.

FIG. 2A depicts, in accordance with some embodiments herein, an example of a double-stranded enzyme recognition nucleic acid molecule. FIG. 2A discloses SEQ ID NOS: 2 and 3, respectively, in order of appearance.

FIG. 2B depicts, in accordance with some embodiments herein, an example of the double-stranded enzyme recognition nucleic acid molecule after enzyme treatment. FIG. 2B discloses SEQ ID NOS 4 and 5, respectively, in order of appearance.

FIG. 3 depicts, in accordance with some embodiments herein, an example of a method for generating a circular nucleic acid molecule.

FIG. 4 depicts, in accordance with some embodiments herein, an example of a workflow of generating a circular nucleic acid library.

FIG. 5 depicts, in accordance with some embodiments herein, an example of a method for generating circular nucleic acid molecules.

FIG. 6A depicts, in accordance with some embodiments herein, an example of a method for generating a circular nucleic acid molecule with transposase.

FIG. 6B depicts, in accordance with some embodiments herein, an example of a method of generating two circular nucleic acid molecules using transposase.

FIG. 7 depicts, in accordance with some embodiments herein, another example of a method of generating two circular nucleic acid molecules using transpose.

FIG. 8 depicts, in accordance with some embodiments herein, an example of a method for amplifying a library using rolling circle amplification in solution.

FIG. 9 depicts, in accordance with some embodiments herein, an example of a method for amplifying a library using rolling circle amplification where the circle is immobilized.

FIG. 10A depicts, in accordance with some embodiments herein, an example of sequencing signals generated by the method disclosed herein.

FIG. 10B depicts, in accordance with some embodiments herein, an example of sequencing signals generated by ligation based circulation.

FIG. 10C depicts an example of sequencing signals generated of an uncircularized library.

FIG. 11 shows a computer control system that is programmed or otherwise configured to implement methods provided herein.

FIG. 12A depicts, in accordance with some embodiments herein, a method of amplifying a circular DNA library.

FIG. 12B depicts, in accordance with some embodiments herein, the results of three rounds of sequencing.

FIG. 13A depicts, in accordance with some embodiments herein, a comparison of read intensity of primer hybridization after hybridization and primer hybridization during amplification.

FIG. 13B depicts, in accordance with some embodiments herein, the results of three rounds of sequencing.

FIG. 14 depicts, in accordance with some embodiments herein, the processivity of the polymerase during sequencing.

FIG. 15 depicts, in accordance with some embodiments herein, the on flow cell (On-FC) circularization of nucleic acids.

FIG. 16 depicts, in accordance with some embodiments herein, the percentage of polonies that provide a signal high enough quality for sequencing (pass filter rate percent) versus polony density.

FIG. 17 depicts, in accordance with some embodiments herein, process flow diagrams for circularization in solution and circularization on flow cell

FIG. 18A depicts, in accordance with some embodiments herein, the polony density of a library circularized on the surface (“linear”) and a library circularized in solution (“circular”).

FIG. 18B depicts, in accordance with some embodiments herein, the library input of a library circularized on the surface (“linear”) and a library circularized in solution (“circular”).

FIG. 19 depicts, in accordance with some embodiments herein, an example polony image using the sequencing methodologies described herein.

FIG. 20 depicts, in accordance with some embodiments herein, error rate of read 1 and read 2 as a function of cycle number.

FIG. 21 depicts, in accordance with some embodiments herein, error rate and polony density on different parts of a flow cell.

FIG. 22 depicts, in accordance with some embodiments herein, pass filter rate percent vs. polony density.

FIG. 23 depicts, in accordance with some embodiments herein, C50 error percent vs. polony density.

FIG. 24A depicts, in accordance with some embodiments herein, a capillary of a flow cell described herein.

FIG. 24B depicts, in accordance with some embodiments herein, a cartridge containing two of the capillaries provided in FIG. 24A.

FIG. 25 depicts, in accordance with some embodiments herein, a fluidics system described herein.

FIG. 26A depicts, in accordance with some embodiments herein, a fluidics system described herein.

FIG. 26B depicts, in accordance with some embodiments herein, a fluidics system described herein.

FIG. 27 depicts, in accordance with some embodiments herein, a cartridge shown in FIG.

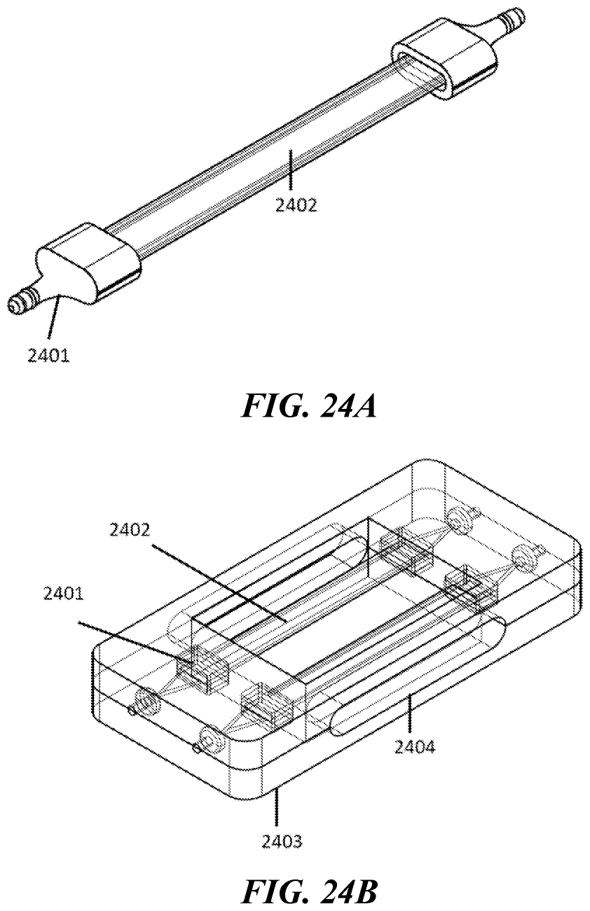

FIG. 24B with a metal plate to regular temperature during use.

FIG. 28 depicts, in accordance with some embodiments herein, a polymer-nucleotide conjugate.

### DETAILED DESCRIPTION

Next generation sequencing (NGS) platforms may begin by isolating DNA or RNA from a biological sample, and, in the case of RNA, converting the RNA to complementary DNA (cDNA) using reverse transcription. Next, the DNA or cDNA may undergo further processing sometimes known as “library preparation” to generate a sequencing library suitable for the NGS platform that will be used. For most NGS platforms, this processing includes fragmenting the DNA or cDNA, such as by enzymatic, mechanical or chemical means. In some cases, certain fragments are enriched, either through size exclusion, polymerase chain reaction (PCR) amplification, or other suitable means. In some cases, adapters are added to one or more of the ends of the fragments. Such adaptors may include amplification handles, sample barcodes or indices, sequencing primers, or unique molecular identifiers (UMIs). In some cases, two asymmetrical adapters are added to either end of the fragment to allow efficient amplification and the introduction of dual indices. Such adapters may include Y-adapters (e.g., two strands with a pairing end and a non-pairing end), or enzyme-cleavable hairpin adapters (e.g., a 1-piece, partially annealed adapter).

However, existing methodologies for adding such adaptors (e.g., Y-adaptors, hairpin adaptors) or otherwise preparing a sequencing library suffer from key disadvantages that limit their widespread application. For example, Y-adapters require the annealing of two DNA oligonucleotides (e.g., the Y-adaptors). Poorly annealed adapters result in low efficiency ligation and low DNA-library conversion. Additionally, the single-stranded portion of a Y-adapter can bind to specific sequences in many genomes unintentionally, leading to depletion of particular sequences and PCR bias. While the 1-piece hairpin adapter can resolve this challenge, this strategy requires an additional enzymatic cleavage step to release the two strands of DNA or cDNA used as the template to be effectively amplified during the PCR amplification step. Thus, there exists a need for nucleic acid sequencing library preparation methods and systems that do not require PCR and do not require additional enzymatic steps in the library preparation process.

In addition, existing NGS workflows involve sequencing both strands of a double-stranded DNA or cDNA molecules, sometimes known as the forward (Read 1) and reverse (Read 2, and the reverse complement to Read 1) strands to ensure sequencing accuracy, good signal quality, and high throughput multiplexity. Such methodologies are sometimes known as “paired-end” sequencing. However, most NGS workflows require a new template to be generated after sequencing of the Read 1 in these libraries, to allow the Read 2 to be sequenced. Re-synthesis of the new template during the sequencing process can take one hour or more before Read 2 sequencing can start. Failure to sequence Read 2 can lead to suboptimal genetic information, as it creates bioinformatics difficulty. Thus, there exists a need for optimized next-generation paired-end sequencing strategies that do not require the re-synthesis of the reverse complement strand (Read 2), thereby reducing reaction times and maintaining improved accuracy.

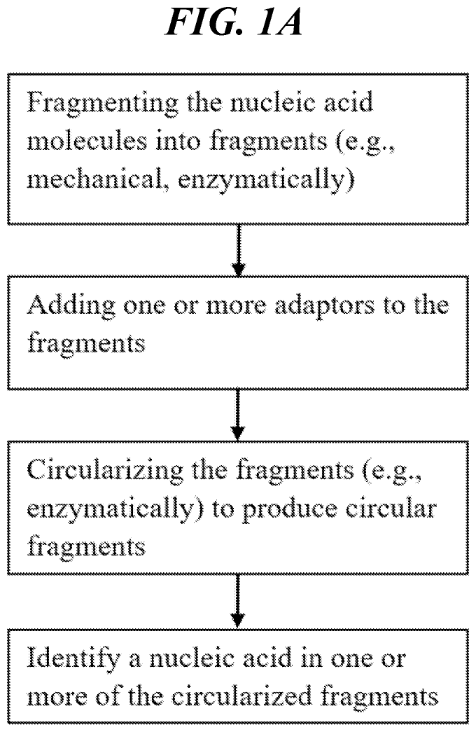

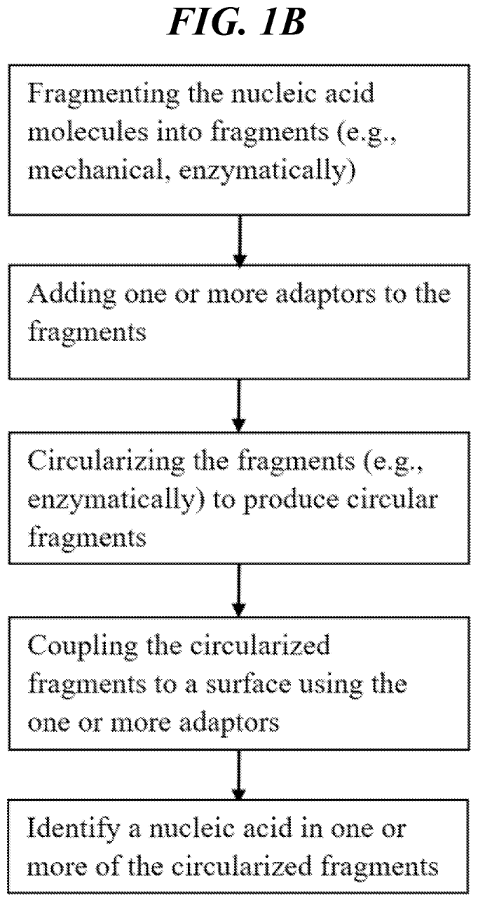

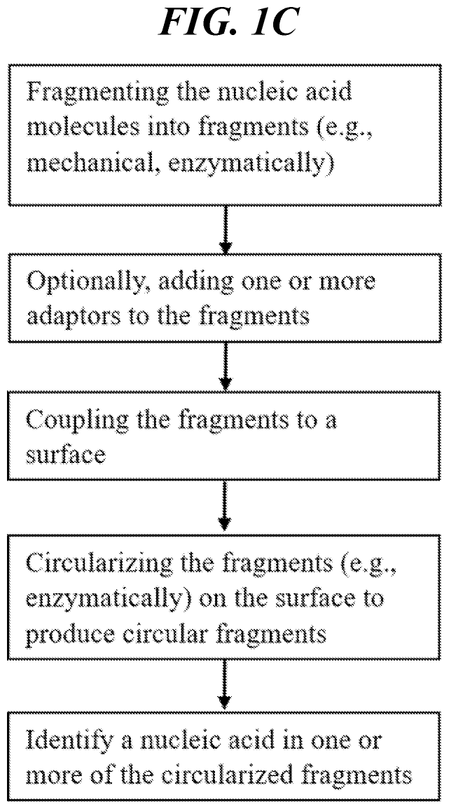

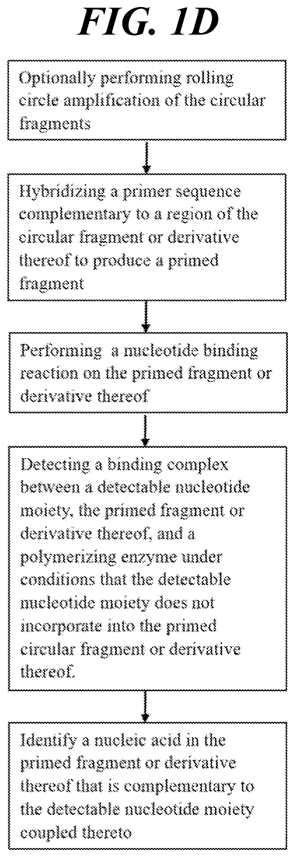

Provided herein are nucleic acid library preparation methods, systems and kits for sequencing (e.g., paired-end sequencing) of a target nucleic acid molecule on a surface that are faster, more efficient, and yield improved accuracy than existing nucleic acid library preparation and sequencing methods. FIGS. 1A-1D illustrates by example some of the methods provided herein. FIG. 1A shows one embodiment, where the method includes fragmenting nucleic acid molecules to produce smaller nucleic acid molecule fragments; adding one or more adaptors to the fragments; circularizing the fragments; and identifying the nucleic acid sequences of the circularized fragments (e.g. sequencing). In some embodiments, as shown in FIG. 1B, the circularization of the fragments is performed in-solution (before coupling the fragments to the surface). In some embodiments, as shown in FIG. 1C, the circularization of the fragments is performed on the surface. In such a case, adding the one or more adaptors may be optional. In some embodiments, at least a portion of the nucleic acid sequence of the fragment is complementary to a splint nucleic acid molecule coupled to the surface. Following addition of the fragment to the surface, the fragment and the splint nucleic acid molecule hybridize, and the splint ligation reaction follows upon addition of a ligating enzyme. In some embodiments, as illustrated by FIG. 1D, the method further includes performing rolling circle amplification of the circular fragments to produce derivatives of the circular fragments; hybridizing a primer sequence complementary to a region of the circular fragment or the derivative to produce a primed fragment; performing a nucleotide binding reaction on the primed fragment or the derivative; detecting a binding complex between a detectable nucleotide moiety, the primed fragment or the derivative, and a polymerizing enzyme under conditions that the detectable nucleotide moiety does not incorporate into the primed circular fragment or derivative thereof; and identifying a nucleotide in the primed fragment or derivative thereof that is complementary to the detectable nucleotide moiety coupled to the nucleic acid.

### Obtaining Nucleic Acids

Provided herein, in some embodiments, are methods for obtaining nucleic acids. In some embodiments, a method for obtaining nucleic acids may comprise lysing one or more cells of a biological sample to create a lysate. In some embodiments, the method may comprise lysing one or more viruses. In some embodiments, lysing may comprise a physical method, a chemical method, an enzymatic method, or any combinations thereof. In some embodiments, the physical method may be grinding. In some embodiments, the physical method is a method that mechanically lyses cells or viruses. In some embodiments, the chemical method may comprise using a detergent. In some embodiments, the detergent may be sodium dodecyl sulfate. In some embodiments, the detergent may be Triton X-100. In some embodiments, the chemical method may comprise using chaotropes. In some embodiments, the chaotrope may comprise guanidine salts. In some embodiments, the chaotrope may comprise an alkaline solution. In some embodiments, the chemical method may comprise a method that uses a chemical to lyse cells or virus.

In some embodiments, the method may comprise separating nucleic acids from non-nucleic acid chemicals. In some embodiments, the separation may comprise centrifugation. In some embodiments, the separation may comprise filtration. In some embodiments, the separation may comprise flowing through a packed bed. In some embodiments, the separation may comprise any method that separates nucleic acid from non-nucleic acid chemicals. In some embodiments, the method may comprise using purification chemistry. In some embodiments, the purification method may isolate DNA. In some embodiments, the purification method may isolate RNA. In some embodiments, the purification method may isolate double-stranded DNA. In some embodiments, the purification method may isolate double-stranded RNA. In some embodiments, the purification method may isolate double stranded nucleic acids that are hybridizations between single-stranded DNA with single-stranded RNA. In some embodiments, the purification method may isolate single-stranded nucleic acids. In some embodiments, the purification method may isolate single-stranded DNA. In some embodiments, the purification method may isolate single-stranded RNA. In some embodiments, the purification method may isolate a single-stranded DNA-RNA hybrid. In some embodiments, the purification method may isolate double-stranded nucleic acids. In some embodiments, the purification method may isolate single-stranded nucleic acids. In some embodiments, the purification method may comprise any method that purifies a specific portion of the genome of the biological material. In some embodiments, the method may comprise selectively sampling a portion of the genome of the biological material. In some embodiments, the selectively sampling method may selectively sample parts of the genome that comprise a target nucleic acid sequence. In some embodiments, the selectively sampling method may selectively sample parts of the genome that are accessible by an enzyme. In some embodiments, the enzyme is a transposase. In some embodiments, the method may comprise washing a solution comprising nucleic acids with a solvent. In some embodiments, the solvent may comprise alcohols. In some embodiments, the nucleic acids described herein are obtained or derived from a biological sample.

In some embodiments, the method for obtaining nucleic acids may comprise synthesizing nucleic acids. In some embodiments, the synthetic method for obtaining nucleic acids may comprise an amplification procedure. In some embodiments, the amplification procedure is PCR. In some embodiments, the amplification procedure is an epigenomic amplification procedure, such as ATAC-Seq.

In some embodiments, the amplification procedure is MNase-seq. In some embodiments, the amplification procedure is ChIP-seq. In some embodiments, the amplification procedure is DNAse-seq. In some embodiments, the amplification procedure is a ligase chain reaction. In some embodiments, the amplification procedure is a transcription-mediated amplification. In some embodiments, the synthetic method may comprise oligonucleotide synthesis. In some embodiments, the synthetic method may comprise annealing based connection of oligonucleotides. In some embodiments, the synthetic method may comprise endonuclease-mediated assembly. In some embodiments, the synthetic method may comprise site-specific recombination. In some embodiments, the synthetic method may comprise long-overlap-based assembly. In some embodiments, the nucleic acids are synthesized.

In some embodiments, the nucleic acid comprises DNA. In some embodiments, the nucleic acid comprises a denatured DNA molecule or fragment thereof. In some instances, the nucleic acid comprises DNA selected from: genomic DNA, viral DNA, mitochondrial DNA, plasmid DNA, amplified DNA, circular DNA, circulating DNA, cell-free DNA, or exosomal DNA. In some embodiments, the DNA is single-stranded DNA (ssDNA), double-stranded DNA, denaturing double-stranded DNA, synthetic DNA, and combinations thereof. The circular DNA may be cleaved or fragmented. In some embodiments, the nucleic acid comprises RNA. In some embodiments, the nucleic acid comprises fragmented RNA. In some embodiments, the nucleic acid comprises partially degraded RNA. In some embodiments, the nucleic acid comprises a microRNA or portion thereof. In some embodiments, the nucleic acid comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Provided herein are methods for obtaining a nucleic acid from a biological sample. In some embodiments, the nucleic acid is extracted from the biological sample. In some embodiments, the extracted nucleic acid is purified to, for example, separate the nucleic acids from other components of the biological sample. In some embodiments, the nucleic acid undergoes one or more of: shearing, cleavage, digestion, or fragmentation steps to obtain a plurality of nucleic acid fragments (e.g., template molecules) of a desired average length, polyadenylation steps, adapter ligation steps to attach adapter sequences to a first and/or second end of the nucleic acid template molecules, library amplification steps, target sequence capture and/or purification steps, or any combination thereof.

### Fragmenting Nucleic Acids

Provided here, in some embodiments, are methods for fragmenting a nucleic acid that has been obtained. In some embodiments, fragmenting comprises at least one of shearing, sonicating, restriction digesting, sequence specific endonuclease treatment, sequence-independent endonuclease treatment and chemical digesting, as well as other shearing approaches. Various shearing options include acoustic shearing, point-sink shearing, and needle shearing. In some steps, the restriction digesting is the intentional sequence specific breaking of nucleic acid molecules. One type of the restriction digesting is an enzyme-based treatment to fragment the double-stranded nucleic acid molecules either by the simultaneous cleavage of both strands, or by generation of nicks on each strand of the double-stranded nucleic acid molecules to produce double-stranded nucleic acid molecules breaks. One type of sonication subjects nucleic acid molecules to acoustic cavitation and hydrodynamic shearing by exposure to brief periods of sonication. As one type of shearing, the acoustic shearing transmits high-frequency acoustic energy waves to nucleic acid molecules. As another type of shearing, the point-sink shearing uses a syringe pump to create hydrodynamic shear forces by pushing a nucleic acid library through a small abrupt contraction. As yet another type of shearing, the needle shearing creates shearing forces by passing DNA libraries through small gauge needle. After the fragmenting, some of the double-stranded nucleic acid fragments contain a region of a nucleic acid sequence with at least about 20, 30, 40, 50, 60, 70, 80, 90, 100, 110, 120, 130, 140, 150, 160, 170, 180, 190, 200, 225, 250, 300, 350, 400, 450, 500, 550, 600 bp or more. In some cases, after the fragmenting, some of the double-stranded nucleic acid fragments contain a region of a nucleic acid sequence with less than about 20.

In some embodiments, the fragmenting further comprises end repair, sticky end generation, and overhang generation. One type of the overhang generation comprises 5′ end generation. One type of the overhang generation comprises 3′ end generation. Some of the steps, such as end repair, sticky end generation, or overhang generation are performed in a tube. Some of the steps, such as such as end repair, sticky end generation, or overhang generation are performed with a solution containing the double-stranded nucleic acid fragments, end repair buffer, and end repair enzyme.

### Adding Adaptors to Nucleic Acid Fragments

Adapters are nucleic acid molecules with known or unknown sequence. Adapters are variously attached to the 3′ end, 5′ end, or both ends of a nucleic acid molecule (e.g. target nucleic acid). Adapters comprise known sequences and/or unknown sequences. Double-stranded and single-stranded adapters are both compatible with various embodiments of the present disclosure. Some of the adapters comprise a barcode (e.g. unique identifier sequence). In some cases, adapters are amplification adapters. The amplification adapters attach to a target nucleic acid and help the amplification of the target nucleic acid. For example, a given amplification adapter comprises one or more of: a primer binding site, a unique identifier sequence, a non-unique identifier sequence, and a surface binding site. In some cases, a target nucleic acid molecule attached with at least one amplification adapter is immobilized on a surface.

In some embodiment, an amplification primer hybridizes to the adapter to be extended using the target nucleic acid molecule as a template in an amplification reaction. Unique identifiers in an adapter may be used to label the amplicons. Some of the adapters are sequencing adapters. Some of the sequencing adapters attach to a target nucleic acid and help the sequencing of the target nucleic acid molecule. For example, a sequencing adapter comprises one or more of: a sequencing primer binding site, a unique identifier site, a non-unique identifier site, and a surface binding site. Some of the target nucleic acid molecules attached with a sequencing adapter are immobilized on a surface on a sequencer. Some of the sequencing primers hybridize to the adapter to be extended using the target nucleic acid molecule as a template in a sequencing reaction. Unique identifiers in an adapter are used in some cases to label the sequence reads of different target sequences, thus allowing high-throughput sequencing of a plurality of target nucleic acid molecules.

Adapters recognize or are complementary to a primer, such as a universal primer. Alternately or in combination, some adapters are specific to a sequencing method. Some of the adapters are single-stranded oligonucleotide added to the ends of the double-stranded target nucleic acid molecule before the joining Some of the adapters are double-stranded oligonucleotide added to the ends of other nucleic acid molecules. Some of the adapters are synthesized to have blunt ends to both terminals. Some of the adapters are synthesized to have sticky end at one end and blunt end at the other. Some of the adapters are synthesized to have sticky end to both terminals.

As mentioned above, adapters may comprise a universal primer site, a surface binding site, or an index site. Some of the adapters comprise at least two of the universal primer sites, the surface binding site, and the index site. Some of the adapters comprise the universal primer site, the surface binding site, and the index site. Some of the universal primer sites comprise one or more universal primers. Some of the universal primers are PCR/sequencing primers that bind to a sequence found in a plurality of plasmid cloning vectors. Some of the universal primer sites comprise one or more amplification primers. Some of the universal primers comprise one or more nucleic acid molecules that are complementary to one or more amplification primers. Some of the universal primer sites comprise one or more nucleic acid molecules that are complementary to one or more universal primers. Some of the surface binding sites are complementary to binding regions covalently attached to a surface. Some of the surface binding sites are configured to immobilize the circular nucleic acid molecules to the surface. After immobilization, the circular nucleic acid molecules are amplified.

In some embodiments, index sites comprise one or more index primers. Some of the index primers enable multiple samples to be sequenced together on the same instrument flow cell or chip. One of such index primers has at least 6 bases, 7 bases, 8 bases, 9 bases, 10 bases or greater. Smaller index primers are also contemplated. Some of the adapters contain single or dual sample indexes depending on the number of libraries combined and the level of accuracy desired. Some of the adapters contain unique molecular identifiers to increase error correction and accuracy. Some of the unique molecular identifiers are short sequences that incorporate a unique barcode onto each molecule within a given sample library. Some of the unique molecular identifiers reduce the rate of false-positive variant calls and increase sensitivity of variant detection. Some of the adapters containing the unique molecular identifiers are xGen Dual Index UMI adapters. Some of the adapters comprise platform-specific sequences for fragment recognition by a sequencer. Some of the platform-specific sequences comprise the P5 and P7 sequences enabling library fragments to bind to the flow cells.

In some embodiments, the adapters are inserted between the double-stranded enzyme recognition nucleic acid molecule and the double-stranded target nucleic acid molecule by a transposase. One type of the transposase is an enzyme that binds to the end of a transposon and catalyzes the movement of the transposon to another part of a nucleic acid molecule. Such movement is performed by a cut and paste mechanism or a replicative transposition mechanism. One type of the transposase is Tn5 transposase. Some of the adapters are ligated to the double-stranded target nucleic acid molecule by a ligase before the joining One type of the ligase is a DNA ligase.

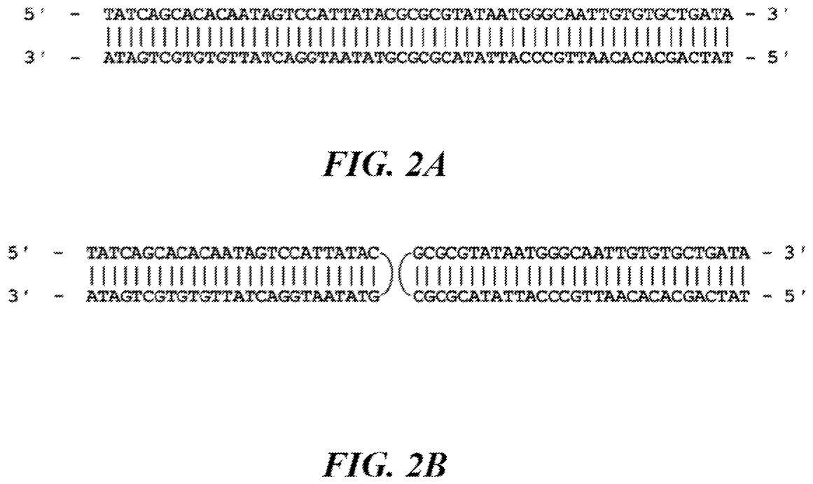

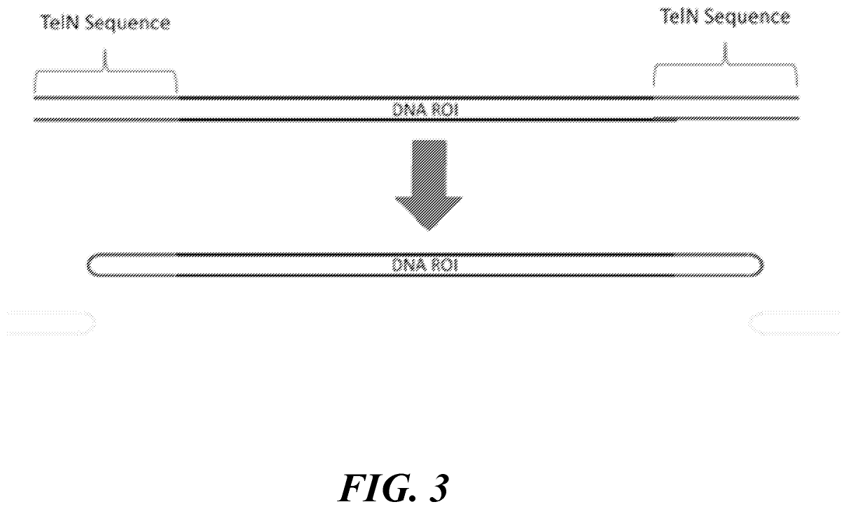

One type of the target double-stranded nucleic acid molecule is a target double-stranded DNA molecule. In the illustrated example of FIG. 2, to create a circular DNA molecule with the target double-stranded DNA molecule, a double-stranded enzyme recognition DNA molecule is inserted flanking the target double-stranded DNA molecule. Both ends of the target double-stranded DNA molecule are inserted with the double-stranded enzyme recognition DNA molecule. Then the Te1N protelomerase catalyzes the double-stranded enzyme recognition DNA molecule on both ends of the target double-stranded DNA molecule to produce a circularized DNA molecule with the target double-stranded DNA molecule circularized, as demonstrated in FIG. 2. The circular DNA molecule produced herein can be used as a template to grow monoclonal DNA populations that are spatially resolved and attached covalently to a surface. In some embodiments, the methods disclosed herein ensure that the target nucleic acid molecules are appropriately spaced in the support to favor formation of monoclonal populations of amplified nucleic acid molecule without substantial cross-contamination between different clonal populations.

### Circularizing Nucleic Acid Fragments

Provided herein are methods for generating circular nucleic acid molecules and circular nucleic acid libraries. In some embodiments, the circular nucleic acid molecules are generated using ligation, such as splint-ligation. Some of such methods create circular nucleic acid molecules (e.g., circular DNA molecules) without ligation.

### Nucleic Acid Ligation

Methods, systems and kits described herein, in some embodiments, utilize ligation to circularize a nucleic acid molecule. In some embodiments, the method includes providing a target nucleic acid and a splint nucleic acid molecule, wherein: a 5′ end of the splint nucleic acid molecule is complementary to a segment of the target nucleic acid at the 3′ end, and the 3′ end of the splint nucleic acid molecule is complementary to a 5′ end of the target nucleic acid; and bringing the target nucleic acid into contact with a ligating enzyme under conditions sufficient to ligate the 5′ end and 3′ of the target nucleic acid molecule. In some embodiments, the splint nucleic acid hybridizes with the 3′ end and the 5′ end of the target nucleic acid sequence, thus forming a temporary circular loop. In some embodiments, the ligating enzyme catalyzes the phosphodiester bond formation between the 3′-phosphate end and the 5′-hydroxyl of the target nucleic acid sequence which results in a primed circular nucleic acid sequence. In some embodiments, the enzyme is a ligase or an enzymatically-active fragment thereof. In some embodiments, the enzyme may be T4 ligase, DNA ligase I, DNA ligase III, DNA ligase IV, T7 ligase, T3 ligase, E. coli ligase, a variant of any one these. In some embodiments, the ligating enzyme or enzymatically-active fragment thereof is a thermostable ligase. In some embodiments, the synthetic ligating enzyme or enzymatically-active fragment thereof comprises an ATP-dependent double stranded DNA specific ligase. In some embodiments, the ligating enzyme or enzymatically-active fragment thereof is derived from a bacteriophage selected from the group consisting of T7, T3, and T4. In some embodiments, the ligase or enzymatically-active fragment thereof is synthetic.

The splint nucleic acid or the target nucleic acid may be single-stranded RNA, double-stranded RNA, single-stranded DNA, double-stranded DNA, single-stranded RNA/DNA hybrid, double-stranded RNA/DNA hybrid, nucleic acid with canonical nucleotides, nucleic acid with non-canonical nucleotides, nucleic acid with both canonical and non-canonical nucleotides, nucleic acid attached to non-nucleic acid components, or any chemical that comprises a nucleic acid sequence. In some embodiments, the splint nucleic acid may be at least about 3 to at least about 10 nucleotides. In some embodiments, the splint nucleic acid may be at least about 10 to at least about 20 nucleotides. In some embodiments, the splint nucleic acid may be at least about 20 to at least about 30 nucleotides. In some embodiments, the splint nucleic acid may be at least about 30 to at least about 40 nucleotides. In some embodiments, the splint nucleic acid may be at least about 40 to at least about 50 nucleotides. In some embodiments, the splint nucleic acid may be at least about 50 nucleotides.

The appropriate conditions for splint ligation may be provided for by an aqueous environment; which may include reagents, pH buffers, and heating, cooling, or maintaining the environment to a predetermined set of temperatures for a predetermined amount of time.

### Ligation-Independent Nucleic Acid Circularization

Methods, systems and kits described herein, in some embodiments, do not utilize ligation. Rather, in some embodiments, an enzyme (e.g., a protelomerase) that identifies a nucleic acid having a target enzyme recognition sequence, cleaves the enzyme recognition nucleic acid molecule at a target site so as to generate an end having a 5′ and 3′ exposed cleavage ends, rejoins 5′ and 3′ cleavage ends of a single exposed end at the target site to form a single linear molecule from the cleaved 5′ and 3′ ends. When this reaction is performed on both ends of a double-stranded nucleic acid molecule having a target molecule added at each end, the result is a circular nucleic acid molecule. In another embodiment, an enzyme (e.g., transposase) transposes 3′ and 5′ end adaptors on a double-stranded nucleic acid molecule thereby circularizing the nucleic acid molecule.

A number of enzymes or enzyme combinations are compatible with this reaction. Often, the enzyme is a protelomerase. One type of protelomerase is Te1N protelomerase, such as that from E. coli phage N1. One type of the enzyme recognizes one or more enzyme recognition nucleic acid molecules attached to random linear double-stranded nucleic acid molecules to create a circular nucleic acid library suitable for sequencing. Some of the libraries generated require clonal amplification of the circular nucleic acid molecules before sequencing process. The use of the enzyme has several advantages to other nucleic acid library preparation methods.

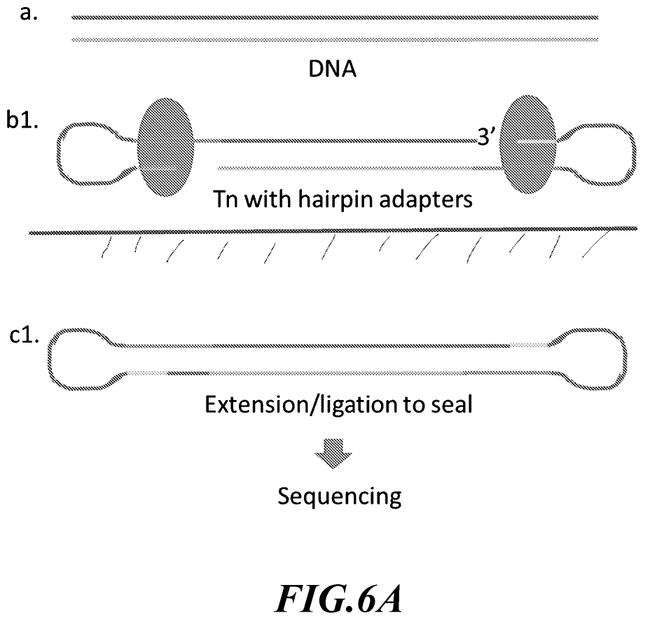

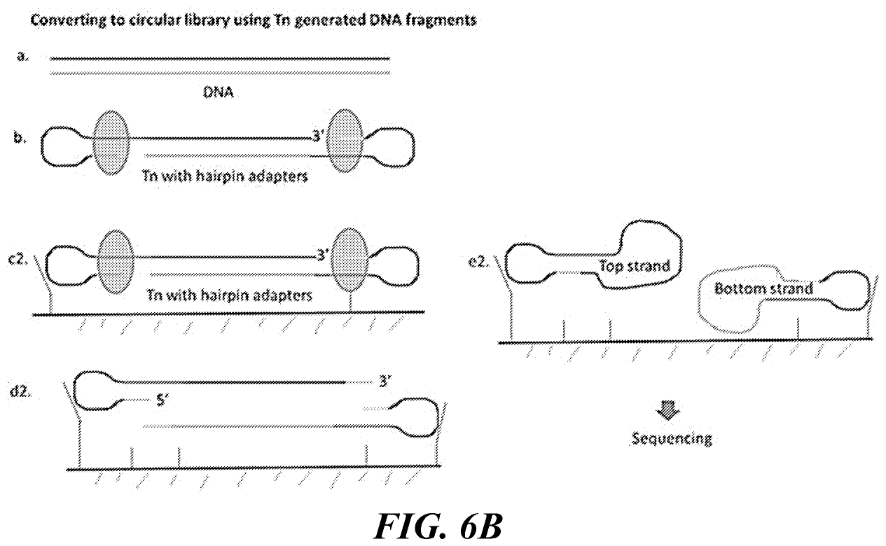

In another example, a transposase is used to transpose (e.g., “cut and paste”) a hairpin adaptor to the 5′ end of the target double-stranded nucleic acid molecule. Two single stranded circular nucleic acid molecules are produced by extension and ligation, as shown in FIG. 6A and FIG. 6B. In some embodiments, a circular nucleic acid molecule (e.g., DNA) library is generated using a transposase (e.g., Transposase 5) to transpose a hairpin adaptor on the 5′ end of a target nucleic acid molecule. In some cases, the transposase or the hairpin adaptor are coupled to the surface directly or indirectly via a surface-bound nucleic acid molecule. Rolling circle amplification can be used to amplify the circular nucleic acid molecule on the surface (e.g., “read 1” or “R1”) thereby generating a reverse complement strand (“read 2” or “R2”), each of which may be sequenced simultaneously to improve accuracy and speed. In some cases, R1 and R2 may be sequenced in read switching intervals of 10 minutes or less. In some cases, R1 and R2 are sequenced simultaneously.

One of such advantages is that the circular nucleic acid molecule contains both the forward and reverse sequences of a target nucleic acid molecule or nucleic acid region of interest. If the circular nucleic acid molecule contains both the forward and reverse sequences, it eliminates the process to synthesize a complementary strand to obtain “paired-end” information. In some embodiments, both 5′ flanking regions to the target nucleic acid molecule contains different sequences and can be hybridized with different sequencing primers to obtain paired-end sequencing reads. Such method eliminates the process to resynthesize and linearize DNA strands between Read 1 and Read 2 to obtain paired end information. Some of the methods disclosed herein simplify a library preparation workflow by removing several reagents used for re-synthesis and decrease overall runtime.

Another advantage is that some of the methods disclosed herein are more efficient than other nucleic acid library preparation methods. These methods suffer from several drawbacks: (1) multiple steps (e.g., high temperature annealing of nucleic acid splint followed by low temperature ligation); (2) inefficiency (e.g., ligation rarely goes to completion in a realistic amount of time amenable for nucleic acid sequence library preparation); (3) incomplete reaction (e.g., preexisting ligation-based circularization rarely results in complete circularization of library strands resulting in loss of a significant fraction of the initial target nucleic acid). Methods disclosed herein, on the other hand, allow library generation in as few as 5 minutes or less, such as 1 hour, 45 minutes, 30 minutes, 25 minutes, 20 minutes, 15 minutes, 10 minutes, 9 minutes, 8 minutes, 7 minutes, 6 minutes, 5 minutes, or no more than 5 minutes, or any time period within the range defined by this list. Alternatives may run longer. Consistent with this rapid library generation, library generation as disclosed herein may be performed isothermally, such as in PCR compatible or other regularly available enzyme buffers.

### Protelomerase-Mediated Circularization

Disclosed herein, in some embodiments, are methods, systems, and kits for circularizing a double-stranded nucleic acid molecule using a protelomerase. In some embodiments, the protelomerase cuts the double-stranded nucleic acid molecule at an enzyme-recognition sequence and leaves covalently closed ends between the forward and reverse strands of the double-stranded nucleic acid molecule, as shown in FIG. 2A and FIG. 2B.

In some embodiments, the protelomerase cleaves the double-stranded enzyme recognition nucleic acid molecule and, after the cleavage, rejoins cleavage ends of the double-stranded enzyme recognition nucleic acid molecule. In some embodiments, the protelomerase cleaves the double-stranded enzyme recognition nucleic acid molecule and, after the cleavage, rejoins cleavage ends of the double-stranded enzyme recognition nucleic acid molecule to form hairpin structures at one or both of the double stranded exposed ends resulting from cleavage of the molecule.

In some embodiments, the protelomerase is Te1N protelomerase. In some embodiments, Te1N circularizes the double-stranded nucleic acid molecule by (a) recognizing the Te1N recognition sequence, (2) catalyzing double-strand hydrolysis at the Te1N recognition sequence thereby producing two double-stranded nucleic acid molecules, and (c) joining the 3′ end of one strand and the 5′ end of the other strand together at both ends of the two double-stranded nucleic acid molecules.

In some embodiments, the joining is carried out by a nucleic acid polymerase during polymerization reactions, such as for example, in a nucleic acid sequencing reaction. In such a case, one or more primer, whether in soluble form or attached to a support, are incubated with a polymerization or extension reaction mix, which may comprise any one or more reagents such as enzyme, dNTPs and buffers. In some cases, the one or more primer is extended through an extension. In some cases, the extension is achieved by an enzyme with polymerase activity or other extension activity. The enzyme may have other activities including 3′-5′ exonuclease activity (proofreading activity) and/or 5′-3′ exonuclease activity. Alternatively, in some embodiments the enzyme can lack one or more of these activities. In some embodiments, the polymerase has strand-displacing activity. Examples of useful strand-displacing polymerases include Bacteriophage Φ29 DNA polymerase and Bst DNA polymerase. In some cases, the enzyme is active at elevated temperatures, e.g., at least 45° C., at least 50° C., at least 60° C., at least 65° C., at least 70° C., at least 75° C., or at least 85° C. One example of a polymerase is Bst DNA Polymerase (Exonuclease Minus), a 67 kDa Bacillus stearothermophilus DNA Polymerase protein (large fragment), exemplified in accession number 2BDP_A, which has 5′-3′ polymerase activity and strand displacement activity but lacks 3′-5′ exonuclease activity. Other polymerases include Taq DNA polymerase I from Thermus aquaticus (exemplified by accession number 1TAQ), Eco DNA polymerase I from Escherichia coli (accession number P00582), Aea DNA polymerase I from Aquifex aeolicus (accession number 067779), or functional fragments or variants thereof, e.g., with at least 80%, 85%, 90%, 95% or 99% sequence identity at the nucleotide level.

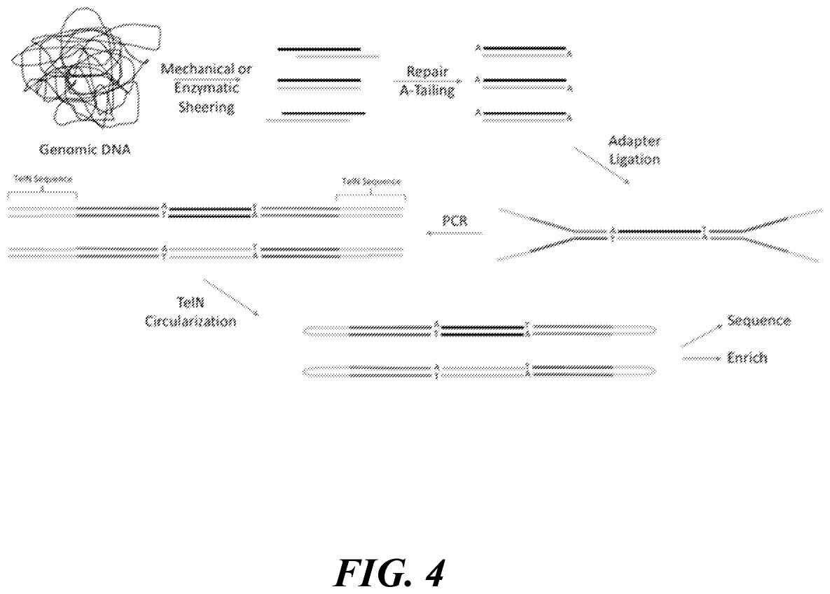

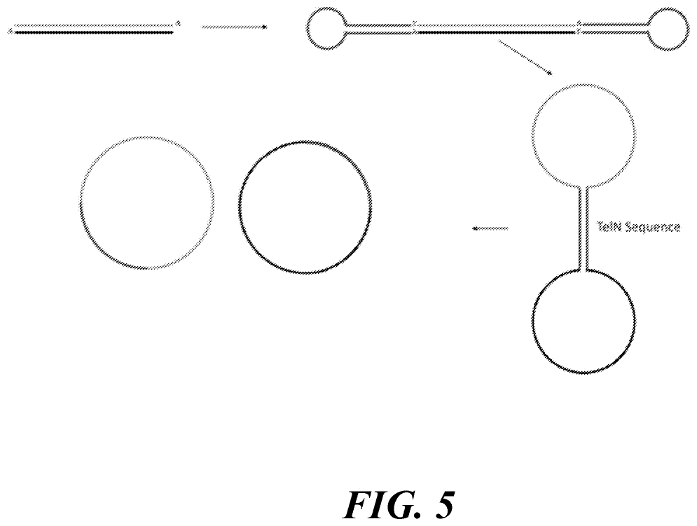

A non-limiting example of a nucleic acid sequencing workflow is provided in FIG. 4. In this example, the double-stranded enzyme recognition nucleic acid sequence is added to the double-stranded nucleic acid molecules by adapter ligation or PCR, if the desired libraries are PCR-free. Such methods comprise preparing a plurality of primers, wherein a given primer of the plurality of primers comprises one strand of the enzyme recognition nucleic acid molecule; annealing the given primer to a single strand of a given double-stranded nucleic acid fragment; extending the given primer to generate a reverse strand of the single strand of the given double-stranded nucleic acid fragment; and creating a forward strand complementary to the reverse strand, wherein the forward strand comprises the single strand of the given double-stranded nucleic acid fragment and the given primer. Some of the forward strands are amplified. Alternatively, the double-stranded enzyme recognition nucleic acid sequence may be added by ligation.

In step one, a double-stranded nucleic acid molecule is sheared mechanically or enzymatically into a plurality of double-stranded nucleic acid fragments. The plurality of double-stranded nucleic acid fragments are 100-5000 bp fragments.

In step two, the plurality of double-stranded nucleic acid fragments is modified. The modification comprises repairing and A-tailing by polymerase. The process of A tailing is performed by adding adenine to 3′ end of each of the plurality of double-stranded nucleic acid fragments.

In step three, one or more adapters are ligated onto the A-tailed double-stranded nucleic acid fragments. The one or more adapters are ligated onto the both ends of A-tailed double-stranded nucleic acid fragments. The one or more adapters comprise a universal primer site, a surface binding site, a P5 site, a P7 site, or an index site. The double-stranded enzyme recognition nucleic acid molecules are inserted at both ends of the adapter-ligated A—tailed double-stranded nucleic acid fragments to form joint double-stranded nucleic acid molecules.

In step four of some workflows, PCR is used to amplify the joint double-stranded nucleic acid molecules.

In step five, Te1N protelomerase is added to the reaction to generate the circular nucleic acid sequence library. The circular nucleic acid sequence library is then purified by Solid Phase Reversible Immobilization (SPRI). The purification process uses SPRI magnetic beads. The magnetic beads are coated with carboxyl groups that can reversibly bind to the circular nucleic acid sequences. The magnetic beads are formulated to specifically bind to the circular nucleic acid sequences and purify out unwanted excess primers, adapter dimers, and salts and enzymes from a wide variety of reactions.

If less PCR cycling is desired, the PCR step (step four) is replaced with an end-elongation step. The end elongation step anneals primers to the joint double-stranded nucleic acid molecules and extends in both 3′ directions completing the joint double-stranded nucleic acid molecules without introducing significant PCR bias.

Some methods disclosed herein comprise separating the plurality of circular nucleic acid molecules before any amplification steps. One type of the separating is performed with separation material. One type of the separation material comprises a plurality of beads. Another type of the separation material comprises an array, such as an array of wells or an array of beads. Some of the separation material comprises a column, such as a packed column, a size-exclusion column, a magnetic column, or any combination thereof. In some embodiments, the separation material comprises a bead, a capillary, a plate, a membrane, a wafer, a well, a plurality of any of these, an array of any of these, or any combination thereof. Some of the separation material positively selects a circular nucleic acid molecule of interest by associating the circular nucleic acid molecule of interest with the separation material. Some of the separation material negatively selects for a circular nucleic acid molecule of interest by associating other circular nucleic acid molecules of a sample with the separation material.

The circular nucleic acid libraries disclosed herein comprise at least 1, 10, 100, 1000, 10000, 100000 or more than 100000 distinct circular nucleic acid molecules. Some of the circular nucleic acid libraries comprise between about 1 to 100000, 10 to 10000, or 100 to 1000 circular nucleic acid molecules with distinct sequences.

In some embodiments, the circular nucleic acid molecules comprise at least one adapter between the enzyme recognition nucleic acid molecule and the double-stranded nucleic acid fragment. Some of the adapters are single-stranded oligonucleotide added to the ends of the double-stranded nucleic acid fragment. Some of the adapters are double-stranded oligonucleotide added to the ends of other nucleic acid molecules. Some of the adapters are synthesized to have blunt ends to both terminals. Some of the adapters are synthesized to have sticky end at one end and blunt end at the other. Some of the adapters are synthesized to have sticky end to both terminals. Some of the adapters comprise a universal primer site, a surface binding site, or an index site. The universal primer site, the surface binding site, and the index site are described elsewhere herein. Some of the adapters contain unique molecular identifiers to provide the highest levels of error correction and accuracy. Some of the unique molecular identifiers are short sequences that incorporate a unique barcode onto each molecule within a given sample library. Some of the unique molecular identifiers reduce the rate of false-positive variant calls and increase sensitivity of variant detection. Some of the adapters containing the unique molecular identifiers are xGen Dual Index UMI adapters. Some of the adapters comprise platform-specific sequences for fragment recognition by a sequencer. Some of the platform-specific sequences comprise the P5 and P7 sites enabling library fragments to bind to the flow cells. The adapters, the universal primer site, the surface binding site, and the index site are described elsewhere herein.

Some of the adapters are inserted between the double-stranded enzyme recognition nucleic acid molecule and the double-stranded target nucleic acid molecule by a transposase. One type of the transposase is an enzyme that binds to the end of a transposon and catalyzes the movement of the transposon to another part of a nucleic acid molecule. Such movement is performed by a cut and paste mechanism or a replicative transposition mechanism. One type of the transposase is Tn5 transposase. Some of the adapters are ligated to the double-stranded nucleic acid molecule by a ligase before the joining.

In some embodiments, the Te1N protelomerase comprises an amino acid sequence of SEQ ID NO: 1. Variants of this sequence, and enzymes having different sequence but comparable enzymatic activity or effecting comparable results when contacted to nucleic acids are also contemplated as consistent with and part of the disclosure herein. The SEQ ID NO: 1 is MSKVKIGELINTLVNEVEAIDASDRPQGDKTKRIKAAAARYKNALFNDKRKFRGKGLQKRITAN TFNAYMSRARKRFDDKLHHSFDKNINKLSEKYPLYSEELSSWLSMPTANIRQHMSSLQSKLKEI MPLAEELSNVRIGSKGSDAKIARLIKKYPDWSFALSDLNSDDWKERRDYLYKLFQQGSALLEEL HQLKVNHEVLYHLQLSPAERTSIQQRWADVLREKKRNVVVIDYPTYMQSIYDILNNPATLFSLN TRSGMAPLAFALAAVSGRRMIEIMFQGEFAVSGKYTVNFSGQAKKRSEDKSVTRTIYTLCEAKL FVELLTELRSCSAASDFDEVVKGYGKDDTRSENGRINAILAKAFNPWVKSFFGDDRRVYKDSRA IYARIAYEMFFRVDPRWKNVDEDVFFMEILGHDDENTQLHYKQFKLANFSRTWRPEVGDENTR LVALQKLDDEMPGFARGDAGVRLHETVKQLVEQDPSAKITNSTLRAFKFSPTMISRYLEFAADA LGQFVGENGQWQLKIETPAIVLPDEESVETIDEPDDESQDDELDEDEIELDEGGGDEPTEEEGPEE HQPTALKPVFKPAKNNGDGTYKIEFEYDGKHYAWSGPADSPMAAMRSAWETYYS. In some embodiments, the protelomerase comprises an amino acid sequence that is more than or equal to about 90% identical to SEQ ID NO: 1. In some embodiments, the protelomerase comprises an amino acid sequence that is more than or equal to about 91, 92, 93, 94, 95, 96, 97, 98, or 99% identical to SEQ ID NO: 1.

(a) Transposase-Mediated Circularization

Provided herein are methods, systems and kits for generating one or more circular nucleic acid molecules utilizing a transposase. Such methods a method comprise, in some embodiments: (a) providing a double-stranded nucleic acid or fragment thereof (e.g., the target double-stranded nucleic acid molecule), (b) coupling an adapter molecule to a 5′ end of at least one strand of the double-stranded nucleic acid molecule or fragment thereof with a transposase; and (c) adding one or more nucleic acids to the at least one strand of the double-stranded nucleic acid molecule or fragment thereof thereby forming a circular nucleic acid molecule.

In some embodiments, forming the circular nucleic acid occurs in a discrete region of the surface. In some embodiments, the first circular nucleic acid is formed on a first discrete region of a surface and the second nucleic acid is formed on a second discrete region of the surface. In some embodiments, forming the circular nucleic acid molecule occurs in solution. In some embodiments, the circular nucleic acid molecule is a single-stranded circular nucleic acid molecule. In some embodiments, the circular nucleic acid molecule is a double-stranded circular nucleic acid molecule. In some embodiments, at least one strand of the double-stranded nucleic acid or fragment thereof is a forward strand.

In some embodiments, the method further comprises forming the circular nucleic acid molecule. In some embodiments, the method further comprises coupling the adapter molecule to the 5′ end of both strands of the double-stranded nucleic acid or fragment thereof. In some embodiments, the method further comprises forming two circular nucleic acid molecules comprising a first circular nucleic acid molecule and a second circular nucleic acid molecule, wherein said first circular nucleic acid molecule comprises a forward strand of the double-stranded nucleic acid molecule or fragment thereof, and wherein said second circular nucleic acid molecule comprises a corresponding reverse strand of the double-stranded nucleic acid molecule or fragment thereof. In one embodiment, the method further comprises amplifying the circular nucleic acid molecule using rolling circle amplification.

In one embodiment, at least one strand of the double-stranded nucleic acid molecule or fragment thereof is sequenced. In one embodiment, both strands of the double-stranded nucleic acid molecule or fragment thereof is sequenced. In one embodiment, the at least one strand of the double-stranded nucleic acid molecule or fragment thereof is sequenced in 10 minutes or less. In one embodiment, the at least one strand of the double-stranded nucleic acid molecule or fragment thereof is sequenced in about 5, 10, 15, 20, 25, or 30 minutes or less.

In one embodiment, the method further comprises synthesizing a complementary strand comprising a nucleic acid sequence that is the reverse complement to a nucleic acid sequence of the at least one strand of the double-stranded nucleic acid molecule or fragment thereof. In one embodiment, the method further comprises (a) removing the at least one strand of the double stranded nucleic acid molecule or fragment thereof; and (b) sequencing the complementary strand. In one embodiment, the removing in (a) is performed enzymatically. The removing may be performed by a strand-displacing polymerase, including without limitations, a viral polymerase, or by a single stranded DNA binding protein, a helicase, or any enzyme having a helicase or strand displacement activity. In one embodiment, the method further comprises amplifying the circular nucleic acid molecule using rolling circle amplification.

In one embodiment, the method further comprises (a) displacing the complementary strand from the at least one strand of the double-stranded nucleic acid molecule or fragment thereof spatially such that the complementary strand and the at least one strand of the double-stranded nucleic acid molecule or fragment thereof do not anneal; and (b) sequencing the complementary strand and the at least one strand of the double stranded nucleic acid molecule or fragment thereof. In one embodiment, the removing in (a) is performed enzymatically. The removing may be performed by a single-stranded nuclease, such as s1 nuclease or mung bean nuclease, or any enzyme having a single-stranded nuclease activity. In one embodiment, the sequencing of the complementary strand and sequencing of the at least one strand of the double stranded nucleic acid molecule or fragment thereof occurs substantially simultaneously. In one embodiment, the method is performed in half of an amount of time of a comparable sequencing reaction that does not sequence the complementary strand and the at least one strand of the double stranded nucleic acid molecule or fragment thereof simultaneously. In one embodiment, the method further comprises amplifying the circular nucleic acid molecule using rolling circle amplification.

In one embodiment, the sequencing of the complementary strand and sequencing of the at least one strand of the double stranded nucleic acid molecule or fragment thereof occurs substantially sequentially in 20 minutes or less. In one embodiment, the sequencing occurs substantially sequentially in 10, 15, 20, 25, 30, 35, 40, 45, 50, 55, or 60 minutes or less.

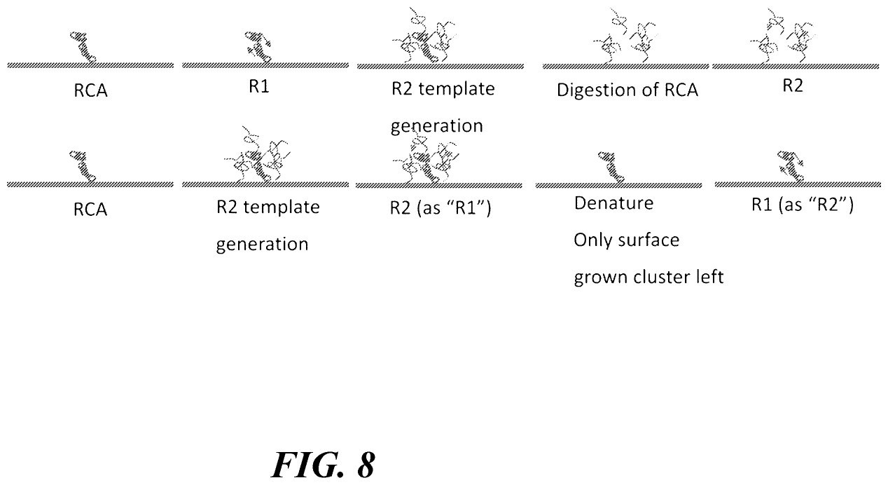

In one embodiment, once the adapter molecules are ligated to the target double-stranded nucleic acid molecule, a circular nucleic acid molecule is formed that contains the forward (“R1”) and reverse strand (“R2”) of the target double stranded nucleic acid molecule. In some embodiments, the circular nucleic acid is single stranded. The circular nucleic acid molecules may be amplified in solution as depicted in FIG. 8. Amplification may occur by rolling circle amplification. Rolling circle amplification may produce an interlinked concatenated circular nucleic acid molecules from the single-stranded circular nucleic acid molecules comprising R1 and R2. The circular nucleic acid molecules produced as a result of may be sequenced by paired end sequencing.

In some embodiments, paired end sequencing of the read 1 (“R1”) and read 2 (“R2”) occurs sequentially. For example, referring to FIG. 8, R1 is generated using rolling circle amplification (“RCA”), R1 is sequenced, an R2 template is generated from R1, and R1 is removed to allow for R2 to be sequenced. In some embodiments, R1 is removed using enzymatic digestion. In this case, R1 may be re-synthesized to start the process over again. In some embodiments, R1 is displaced by a strand displacement polymerase This method may result in an intensity boost of read 2.

In some embodiments, paired-end sequencing occurs simultaneously. Referring to FIG. 8, the R2 template is generated and instead of removing R1 by enzymatic digestion, R1 and R2 are displaced to different discrete regions and sequenced simultaneously.

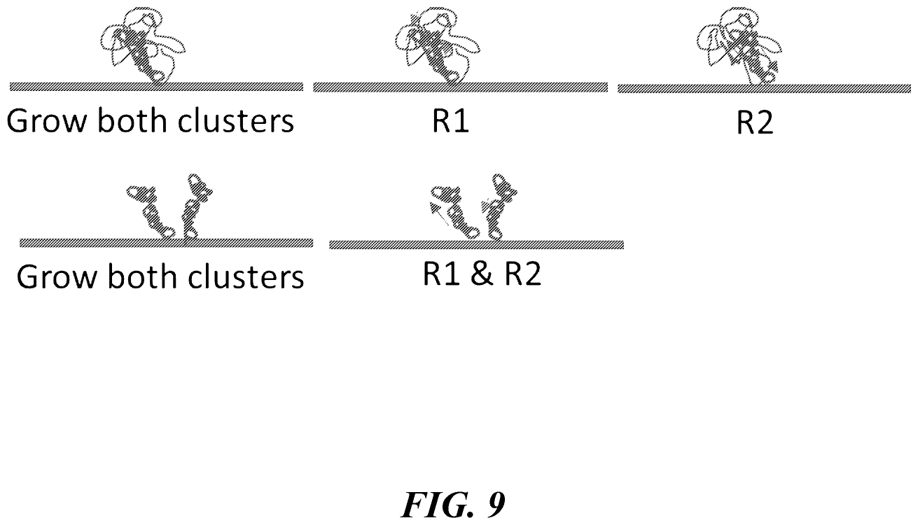

In another embodiment, the circular nucleic acid molecule described herein is coupled to a surface by one of the methods described herein. In some embodiments, the circular nucleic acid molecule is amplified on the surface by rolling circle amplification, as depicted in FIG. 9. Referring to FIG. 9, R1 is coupled to the surface, and is used to generated R2. In this example both R1 and R2 are sequenced on the surface using paired-end sequencing sequentially or simultaneously. This method does not require strand re-synthesis, because R1 is not enzymatically digested.

In some embodiments, the circular nucleic acid molecule is amplified using a strand displacement enzyme resulting in only 1 amplification to produce both strand contents and requiring no strand re-synthesis.

Methods disclosed herein also comprise identifying clusters of amplified circular nucleic acid molecules on the surface that are related, e.g., forward strand and corresponding reverse strand. For example, circularization of each strand separately can yield catenated or otherwise spatially associated template molecules which, when captured on a sequencing surface as disclosed herein, will be located in proximity to one another such that by comparing adjacent clusters, the paired sequences may be identified. Similarly, by separately circularizing each strand and capturing the templates on the surface prior to amplification (carrying out, for example, RCA from a surface bound primer) yields vicinal templates whose proximity indicates their relatedness. Primers specific to each strand may be placed in spatial proximity on a surface may be included, with or without prior circularization of each strand, to capture the strands in proximity on the sequencing surface and allow further amplification while retaining spatial proximity from which the relationship between the forward and reverse strands may be identified or inferred. It can be inferred, for example, that strands are related if the clusters from which the signals arise are colocalized within a sequencing surface, such as occupying the same location, occupying spatially unresolvable locations, or occupying positions within 20×, 15×, 10×, 5×, 4×, 3×, 2×, 1, 0.5×, or less of the radius of a particular template cluster or of an average template cluster. By thus linking related molecules, they can then be captured on a sequencing surface in a way that leaves them spatially related such that their relatedness can be deduced from their proximity.

In some embodiments, the method comprises: (a) denaturing a double-stranded enzyme recognition nucleic acid molecule to form two single-stranded enzyme recognition nucleic acid molecules; (b) joining each of the two single-stranded enzyme recognition nucleic acid molecules to each end of a target double-stranded nucleic acid molecule to form a joint nucleic acid molecule, wherein, after the joining, each of the two single-stranded enzyme recognition nucleic acid molecules takes a form of a hairpin; (c) denaturing the joint nucleic acid molecule; (d) hybridizing the two single-stranded enzyme recognition nucleic acid molecules in the joint nucleic acid molecule to form the double-stranded enzyme recognition nucleic acid molecule in the joint nucleic acid molecule; and (e) contacting the joint nucleic acid molecule with an enzyme, wherein the enzyme binds to the double-stranded enzyme recognition nucleic acid molecule to form two circular nucleic acid molecules. In some embodiments, one of the two circular nucleic acid molecules contains a reverse strand that is complementary to a forward strand in another one of the two circular nucleic acid molecules.

In some embodiments, the enzyme cleaves the double-stranded enzyme recognition nucleic acid molecule and, after the cleavage, rejoins cleavage ends of the double-stranded enzyme recognition nucleic acid molecule. In some embodiments, the enzyme cleaves the double-stranded enzyme recognition nucleic acid molecule and, after the cleavage, rejoins cleavage ends of the double-stranded enzyme recognition nucleic acid molecule to form hairpin structures. One type of the enzyme is a protelomerase. One type of the protelomerase is Te1N protelomerase. The Te1N protelomerase is described elsewhere herein.

Some of the joint nucleic acid molecules comprise at least one adapter between the enzyme recognition nucleic acid molecule and the target double-stranded nucleic acid molecule. Some of the adapters are described elsewhere herein. Some of the adapters comprise a universal primer site, a surface binding site, or an index site. The universal primer site, the surface binding site, and the index site are described elsewhere herein. Some of the adapters contain unique molecular identifiers, which are described elsewhere herein. Some of the adapters comprise the P5 and P7 sites enabling library fragments to bind to the flow cells. In some embodiments, the joint nucleic acid molecules do not comprise any adapter between the enzyme recognition nucleic acid molecule and the target double-stranded nucleic acid molecule.

As illustrated in FIG. 6A and FIG. 6B, two complementary single-stranded enzyme recognition nucleic acid molecule are placed the on each end of a target double-stranded nucleic acid molecule by hairpin ligation. A hairpin is a nucleic acid molecule containing both a region of single stranded molecule (a loop region) and regions of self-complementary molecule such that an intra-molecular duplex is formed under hybridizing conditions. Next, an intramolecular circularization is performed to create double-stranded enzyme recognition nucleic acid molecule. Next, Te1N protelomerase catalyzes the double-stranded enzyme recognition nucleic acid molecule to produce two independent circular single-stranded nucleic acid molecules. Each of the circular single-stranded nucleic acid molecules contains reverse complementary strand of another circular single-stranded nucleic acid molecule. In some cases, this method disclosed herein eliminates the duplex region of the target double-stranded nucleic acid molecule. Accordingly, one is able to separately package individual strands of a double-stranded starting molecule into sequencing library constituents.

Some of these methods disclosed herein are compatible with any or all of paired-end read sequencing, indexing, and unique molecular index (UMI) barcoding.

(b) Nucleic Acid Sequencing Adapters

Provided herein are adapters for generating one or more circular nucleic acid molecules. Some of the adapters are Y adapters. Some of the Y adapters comprise at least part of an enzyme recognition nucleic acid molecule, a universal primer site, a surface binding site, and an index site. Some of the Y adapters further comprise a P5 site or a P7 site. One of the Y adapters contains both a region of two single stranded molecules (a fork region) and regions of self-complementary molecule. Some of the regions of self-complementary molecule comprise at least part of an enzyme recognition nucleic acid molecule, a universal primer site, a surface binding site, or an index site. Some of the fork regions comprise at least part of an enzyme recognition nucleic acid molecule, a universal primer site, a surface binding site, or an index site. The adapters, the universal primer site, the surface binding site, and the index site are described elsewhere herein.

In some cases, an enzyme binds to the enzyme recognition nucleic acid molecule. The enzyme cleaves the enzyme recognition nucleic acid molecule and, after the cleavage, rejoins cleavage ends of the enzyme recognition nucleic acid molecule. In some embodiments, the enzyme cleaves the enzyme recognition nucleic acid molecule and, after the cleavage, rejoins cleavage ends of the enzyme recognition nucleic acid molecule to form hairpin structures. One type of the enzyme is a protelomerase. One type of the protelomerase is Te1N protelomerase. The Te1N protelomerase is described elsewhere herein.

Some of the adapters are hairpin adapters. Some of the hairpin adapters comprise at least part of an enzyme recognition nucleic acid molecule, a universal primer site, a surface binding site, and an index site. Some of the hairpin adapters further comprise a P5 site or a P7 site. One of the hairpin adapters contains both a region of single stranded molecule (a loop region) and regions of self-complementary molecule. Some of the regions of self-complementary molecule comprise at least part of an enzyme recognition nucleic acid molecule, a universal primer site, a surface binding site, or an index site. Some of the loop regions comprise at least part of an enzyme recognition nucleic acid molecule, a universal primer site, a surface binding site, or an index site. The adapter, the universal primer site, the surface binding site, and the index site are described elsewhere herein.

In some embodiments, the surface binding site comprises a nucleic acid sequence that is complementary to a surface-bound nucleic acid molecule by Watson-Crick base pairing, by Hoogsteen base pairing, by triplex pairing, by formation of a G-quartet, by incorporation of an affinity tag or epitope, or by other means of obtaining pairwise, 3-way, or 4-way binding of two or more nucleic acid molecules.

In some cases, the adapter molecule comprises at least one unnatural nucleic acid configured to participate in a G-quadruplex. In some embodiments, the surface binding site of the adapter molecule comprises the at least one unnatural nucleic acid. In some embodiments, the adapter molecule comprises 1, 2, 3, or 4 unnatural nucleic acids configured to participate in a G-quadruplex. In some cases, the adapter is bi-molecular, which means that there is at least one unnatural nucleic acid that participates in the G-quadruplex is positioned in at least two nucleic acid molecules (e.g., the surface-bound nucleic acid molecule and the surface binding site of the adapter molecule). In other words, each of the two nucleic acid molecules comprises half of the G-quadruplex and interaction between the two halves of the G-quadruplex forms a bond between the two nucleic acid molecules. In some cases, the G-quadruplex is tunable, because interaction between the unnatural nucleic acids that participate in the G-quadruplex can be manipulated with a change in condition, such as depletion of potassium (K+) or heat. The interactions between unnatural nucleic acids in the G-quadruplex, in some cases, is manipulated under different conditions than the conditions used to denature the target double-stranded nucleic acid molecule during, for example, amplification. Thus, in some embodiments described herein the adapter molecule is tunable such that association and disassociation of the one or more circular nucleic acid molecules to the surface is controlled separately from denaturing the circular nucleic acid molecule. At least one of the advantages of the tunable G-quadruplex described herein is that binding and release of the G-quad may be effected under different conditions than the Watson-crick base pairing of other regions of interaction, allowing G-quad regions and W-C base paired regions to be modulated separately. Additionally, G-quads may be maintained in a manner that is significantly more stable than ordinary base pairing interactions, allowing the use of harsher conditions in the flowcell, such as for washing, denaturation, and removal of used or unwanted sequencing templates.

In effect, this allows the adapter to be occluded from interaction with free ends during ligation, and to turn into two separate strands via a condition change (i.e. depletion of K+ or heat to denature the G-quadruplex). The net effect is that this should occur at a higher frequency than with a Y-shaped adapter, as the invention minimizes potential off-target interactions. As this G-quadruplex is split on the two molecules of the adapter, there is no need to use enzymatic cleavage to separate the relevant adapter sections, meaning that no additional reagents/steps are required, and there is no potential risk of cleaving library inserts, resulting in a loss of coverage of particular regions.

In some cases, an enzyme binds to the enzyme recognition nucleic acid molecule. If the enzyme recognition nucleic acid molecule is in the regions of self-complementary molecule, the enzyme cleaves the enzyme recognition nucleic acid molecule and, after the cleavage, rejoins cleavage ends of the enzyme recognition nucleic acid molecule. In some embodiments, the enzyme cleaves the enzyme recognition nucleic acid molecule and, after the cleavage, rejoins cleavage ends of the enzyme recognition nucleic acid molecule to form hairpin structures. One type of the enzyme is a protelomerase. One type of the protelomerase is Te1N protelomerase. The Te1N protelomerase is described elsewhere herein.

In some cases, a transposase is used to transpose the adapter molecule onto the target double-stranded nucleic acid molecule. In some embodiments, the enzyme is a transposase. In some embodiments, the transposase is transposase 5. In some cases, the adapter molecule comprises a transposon that is associated with the transposase, such that transposition of the adapter molecule onto the target double-stranded nucleic acid molecule is performed without a need for polymerase chain reaction.

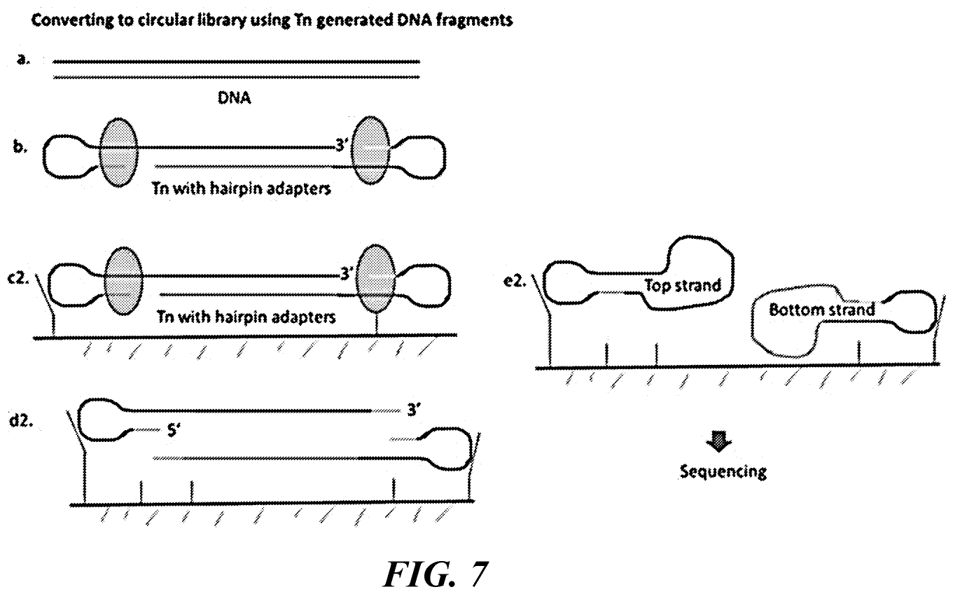

In a non-limiting example, depicted in FIG. 6, hairpin adapter molecules are transposed to the target double-stranded nucleic acid molecule (e.g., DNA). The transposase may be in solution or may be on the hairpin adapter molecules. The hairpin adapters and the target double-stranded nucleic acid molecule undergo extension and ligation to create a circular target nucleic acid molecule, which can then be sequenced. In another embodiment, depicted in FIG. 6, the transposase is used to ligate the hairpin adapters. The ligation may occur in solution. The hairpin adapter molecule may be attached to the solid support. The two strands may then be separately circularized, creating two nearby circular DNA molecules on a solid surface support. In another embodiment, the transposase inserts an adapter sequence or a barcode into the target double-stranded nucleic acid molecule. In another embodiment, depicted in FIG. 7, the transposon with hairpin adapters are immobilized on the surface. The target double-stranded nucleic acid molecule may hybridize and anneal to the hairpin adapters. A splint on the surface may mediate circularization of the top strand and bottom strand of the DNA target molecule, resulting in formation of two nearby circles immobilized to a solid support.

In some embodiments, the adapter molecule is coupled to a surface-bound nucleic acid molecule coupled to a surface. In some embodiments, the adapter molecule is couple to the surface-bound nucleic acid molecule by nucleic acid hybridization. In some embodiments, the surface bound nucleic acid molecule comprises at least one unnatural nucleic acid configured to participate in the G-quadruplex. In some embodiments, the surface-bound nucleic acid molecule comprises a transposon associated with the transposase. In some embodiments, one or more of the transposase and the adapter molecule is coupled to a surface.

### Nucleic Acid Amplification

Some of the methods disclosed herein further comprise amplification of the plurality of circular nucleic acid molecules. Some of the amplifications comprise amplification by polymerase chain reaction (PCR), loop mediated isothermal amplification, nucleic acid sequence based amplification, strand displacement amplification, multiple displacement amplification, rolling circle amplification, ligase chain reaction, helicase dependent amplification, ramification amplification method, or any combination thereof. One type of amplification is clonal amplification of the plurality of circular nucleic acid molecules. One of the clonal amplifications comprises performing rolling circle amplification. In some cases, the amplification comprises at least 2, 3, 4, 5, 6, 7, 8, 9, 10, 11, 12, 13 or greater cycles of amplification.

Any amplification method described herein may comprise repeated cycles of nucleic acid amplification. A cycle of amplification may comprise: (a) hybridization of one or more primers to a template strand or a complement thereof, (b) primer extension to form a first and/or second extended strand, and (c) partial or incomplete denaturation of the extended strand(s) from the template strand(s) or complements thereof, e.g., through the use of a non-thermal duplex destabilizing mechanism, such as the binding of a helicase or a single-stranded DNA binding protein, that shifts the equilibrium between single-stranded and double-stranded nucleic acid molecules towards the single-stranded form. One type of the template is a circular nucleic acid molecule.

Some of the circular nucleic acid molecules are amplified using primers. Some of the primers are supplied in solution or immobilized on a solid support. In some cases, the circular nucleic acid molecules are amplified using primers immobilized on/to one or more solid or semi-solid supports. In some cases, the support comprises immobilized primers that are complementary to a portion of an adapter in the circular nucleic acid molecule. In another example, the support may not significantly comprise an immobilized primer that is complementary to a portion of an adapter in the circular nucleic acid molecule.

In some cases, a plurality of circular nucleic acid molecules is amplified simultaneously in a single continuous liquid phase in the presence of one or more supports, where each support comprises one or more immobilization sites. In some cases, each circular nucleic acid molecule is amplified to generate a clonal population of amplicons, where individual clonal populations are immobilized within or on a different immobilization site from other amplified populations. For example, a different immobilization site can be a different discrete region on a support. In some cases, the amplified populations remain substantially clonal after amplification.

A circular nucleic acid molecule is for example amplified to generate clonal populations which comprise both forward strand and reverse strand of a double-stranded nucleic acid molecule. In an embodiment, clonality is maintained in the resulting amplified nucleic acid populations by maintaining association between circular nucleic acid molecule and its primer immobilized, thereby effectively associating or “tethering” associated clonal progeny together and reducing the probability of cross-contamination between different clonal populations. In some cases, a clonal population of substantially identical nucleic acids has a spatially localized or discrete macroscopic appearance. In an embodiment, a clonal population resembles a distinct spot or colony.

Some of the methods generate a localized clonal population of clonal amplicons, which may be immobilized in/to/on one or more supports. One type of the support is solid or semisolid (such as a gel or hydrogel). The amplified clonal population may be attached to the support's external surface or can also be within the internal surfaces of a support (e.g., where the support has a porous or matrix structure).

In some cases, amplification is achieved by multiple cycles of primer extension along a circular nucleic acid molecule. In some cases, one or more primers are immobilized in/on/to one or more supports. In some cases, one primer is immobilized by attachment to a support. In some examples, a second primer is present and may not be immobilized or attached to a support. In some cases, different circular nucleic acid molecules are amplified onto different supports or immobilization sites simultaneously in a single continuous liquid phase to form clonal nucleic acid populations. One type of the liquid phase is considered continuous if any portion of the liquid phase is in fluid contact or communication with any other portion of the liquid body. In another example, a liquid phase is considered continuous if no portion is entirely subdivided or compartmentalized or otherwise entirely physically separated from the rest of the liquid body. In some cases, the liquid phase is flowable. In some cases, the continuous liquid phase is not within a gel or matrix. In other cases, the continuous liquid phase is within a gel or matrix. For example, the continuous liquid phase occupies pores, spaces or other interstices of a solid or semisolid support.

Where the liquid phase is within a gel or matrix, one or more primers are immobilized on a support. In some cases, the support is the gel or matrix itself. Alternatively, the support is not the gel or matrix itself. In an example one primer is immobilized on a solid support contained within a gel and is not immobilized to gel molecules. The support is for example in the form of a planar surface or one or more microparticles.

For some circular nucleic acid molecules, the first hybridization step comprises hybridizing a primer to the circular nucleic acid molecule for extension. For some circular nucleic acid molecules, the primer extension reaction comprises a step of rolling circle amplification (RCA) in which a strand-displacing polymerase synthesizes a new strand that is a concatemer comprising multiple copies of the nucleic acid molecule and adapter sequences encompassed by the circular nucleic acid molecules. In some cases, the concatemer contains at least one single strand (either forward or reverse strand) of the double-stranded target nucleic acid molecule. In some cases, the concatemer contains both strands (both forward and reverse strands) of the double-stranded target nucleic acid molecule. In some cases, the concatemer further comprises at least one enzyme recognition nucleic acid fragment. In yet another case, the concatemer further comprises at least one adapter between one enzyme recognition nucleic acid fragment and a single strand of the double-stranded target nucleic acid molecule. In some cases, the concatemer contains multiple single strands of the double-stranded target nucleic acid molecule, multiple enzyme recognition nucleic acid molecules, and multiple adapters between each enzyme recognition nucleic acid fragment and each single strand of the double-stranded target nucleic acid molecule. Some of the multiple adapters are separated by at least one single strand of the double-stranded target nucleic acid molecule or at least one enzyme recognition nucleic acid fragment.

In some cases, a given adapter of the multiple adapters comprises multiple surface binding sites, thereby binding to different immobilization sites on a surface. In this situation, the concatemer having the given adapter forms one or more bridge structures on the surface. Some of the bridge structures are then amplified through one or more application process.

Some of the methods are performed under isothermal amplification conditions. Some of the methods performed under isothermal amplification conditions use one or more non-thermal duplex destabilization mechanisms to promote primer hybridization and accelerate the amplification reactions under isothermal conditions. Examples of suitable non-thermal duplex destabilization mechanisms include, but are not limited to, (i) the use of chemical denaturants (e.g., NaOH solutions, high salt concentrations, etc.), (ii) the use of helicase proteins to facilitate the unwinding and separation of double-stranded regions of the nucleic acid molecules during the amplification reaction, (iii) the use of single-stranded DNA-binding proteins (SSBs) to shift the equilibrium between single-stranded and double-stranded nucleic acid molecules towards the single-stranded form during the amplification reaction, and (iv) the use of “thermal breathing” (i.e., fluctuations in the degree of nucleotide base-pairing when the reaction temperature is held fixed at or near the melting temperature, Tm, for duplex nucleic acid molecules). The destabilization of the duplex structure need only occur near the ends of the duplex molecule in order to facilitate primer binding and accelerate the amplification Some of the non-thermal duplex destabilization mechanisms employed comprise the use of at least one helicase, at least one single-stranded DNA binding protein, thermal breathing, or any combination thereof. Some of the methods use one of the non-thermal duplex destabilization mechanisms. Some of the methods use a combination of two or more non-thermal duplex destabilization mechanisms.

Some of the non-thermal duplex destabilization mechanisms allow the amplification process to be performed under isothermal conditions. As used herein, the term “isothermal” indicates that the set of amplification reactions may all be performed within a specified range of a specified set temperature. One type of the thermal breathing-dependent isothermal amplification is performed by maintaining the amplification reaction temperature to be within ±1° C., ±2.5° C., ±5° C., ±7.5° C., or ±10° C. of a specified melting temperature for the circular nucleic acid molecule. One type of isothermal amplification is performed at a set temperature ranging from about 20° C. to about 80° C., or from about 20° C. to about 80° C. In some cases, the specified melting temperature is at least 20° C., at least 25° C., at least 30° C., at least 35° C., at least 40° C., at least 45° C., at least 50° C., at least 55° C., at least 60° C., at least 65° C., at least 70° C., at least 75° C., or at least 80° C. In some cases, the specified melting temperature is at most 80° C., at most 75° C., at most 70° C., at most 65° C., at most 60° C., at most 55° C., at most 50° C., at most 45° C., at most 40° C., at most 35° C., at most 30° C., at most 25° C., or at most 20° C.

Some of the methods for clonal amplification of nucleic acid molecules that comprise the use of one or more non-thermal duplex destabilization mechanisms enable one to achieve improved isothermal amplification rates such that the clonal population increases exponentially with a doubling time of at most 1 hour, 30 minutes, 20 minutes, 10 minutes, or 5 minutes or less. In other cases, the methods for clonal amplification of nucleic acid molecules that comprise the use of one or more non-thermal duplex destabilization mechanisms enable one to achieve improved isothermal amplification rates such that the clonal population increases exponentially with a doubling time of more than 1 hour.

Some of the methods for clonal amplification of nucleic acid molecules that comprise the use of one or more non-thermal duplex destabilization mechanisms enable one to achieve process times or isothermal amplification reaction times (i.e., the total time required to complete the clonal amplification process) of at most 50 minutes, 40 minutes, 30 minutes, 20 minutes, 10 minutes, or 5 minutes or less. In other cases, the methods for clonal amplification of nucleic acid molecules that comprise the use of one or more non-thermal duplex destabilization mechanisms enable one to achieve process times or isothermal amplification reaction times (i.e., the total time required to complete the clonal amplification process) of more than 50 minutes.

Some of the methods disclosed herein comprise sequencing the plurality of circular nucleic acid molecules. Such sequencing comprises bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

Some of the methods disclosed herein take at most about 5 hours, 4 hours, 3 hours, 2 hours, 1 hours, 30 minutes, 20 minutes, 10 minutes, 5 minutes or less to complete. In some cases, some of the methods disclosed herein take more than about 5 hours to complete. Some of the methods disclosed herein take from about 1 minute to 5 hours, 5 minutes to 4.5 hours, 10 minutes to 4 hours, 20 minutes to 3.5 hours, 30 minutes to 3 hours, 1 hour to 2.5 hour, or 1.5 hours to 2 hours to complete.

Some of the methods disclosed herein have higher efficiency to create nucleic acid libraries. Typical ligation based approaches cost 16 hours. Some of the methods disclosed herein take 30 minutes and are able to be optimized down to 5 minutes. Additionally, some of the methods disclosed herein create circular nucleic acid molecules to generate monoclonal, spatially resolved amplicons that demonstrate brighter signals during sequencing processes than circular nucleic acid molecules generated through ligation based approaches. Finally, some of the methods disclosed herein do not present complementary flanking sequencing or generate an entire complement to the library strand of interest that competes with amplification and inhibits amplicon growth.

### Hybridizing the Nucleic Acids to a Surface

Provided herein are methods of coupling a nucleic acid library to a surface, such as a low non-specific binding surface described herein. In some embodiments, coupling occurs before circularization of the library. In some embodiments, coupling occurs after circularization. In either case, a region of the nucleic acid molecule of the library is specific to a surface-bound capture molecule. In some embodiments, the library is amplified prior to coupling the library to the surface. In some embodiments, the library is amplified following coupling the library to the surface.

In some embodiments, nucleic acids in a library are coupled to a surface (e.g., low non-specific binding surface) by way of hybridization between a region of the nucleic acid molecule and a region of a capture molecule coupled to the surface. Unless noted otherwise, hybridization may occur between nucleic acids of any length and the hybridized nucleic acid may take on one or a combination of many structural forms, including, but not limited to: the B-form, the A-form, Z-form, stem loop, pseudoknot, or other hybridization structures formed by base-pairing interactions between two or more single-stranded nucleic acids. In some embodiments, hybridization occurs between two single-stranded nucleic acids of any length. In some embodiments, hybridization occurs between a single-stranded linear nucleic acid and a single-stranded linear nucleic acid. In some embodiments, hybridization occurs between a single-stranded linear nucleic acid and a single-stranded circularized nucleic acid. In some embodiments, hybridization occurs between a single-stranded circularized nucleic acid and a single-stranded circularized nucleic acid. In some embodiments, hybridization occurs between a DNA molecule and a DNA molecule. In some embodiments, hybridization occurs between a DNA molecule and an RNA molecule. In some embodiments, hybridization occurs between an RNA molecule and an RNA molecule. In some embodiments, hybridization occurs between a DNA molecule and a DNA/RNA hybrid molecule. In some embodiments, hybridization occurs between an RNA molecule and a DNA/RNA hybrid molecule. In some embodiments, hybridization occurs between a DNA/RNA hybrid molecule and a DNA/RNA hybrid molecule.

In some embodiments, a nucleic acid molecule of the library is coupled to the surface by hybridization between a nucleic acid sequence of the nucleic acid molecule and one or more capture nucleic acid molecules coupled the surface. In some embodiments, the one or more capture nucleic acid molecules is a splint nucleic acid molecule described herein, and facilitates circularization of the nucleic acid molecule on the surface in the presence of a ligating enzyme or catalytically-active portion thereof described herein.

In some embodiments, the one or more capture nucleic acid molecules (as referred to here as surface-bound primer) hybridizes to one or more adaptors of the nucleic acid molecule, such as an adaptor containing an index sequence disclosed herein. In some embodiments, the index sequence is any unique sequence of 8 to 10 nucleotides, usable as unique index sequence pairs.

### Hybridization Buffers

In some embodiments, the methods and compositions as disclosed herein may comprise or may further comprise the use of one or more hybridization buffers. Said buffers may serve to, for example, reduce the time required to hybridize one or more clusters or nucleic acid molecules to a surface or a surface-bound oligonucleotide, or a solution phase oligonucleotide, such as an adapter oligonucleotide, a capture oligonucleotide, a condenser oligonucleotide, or the like. Said hybridization buffers may or may also, in some embodiments, lead to improved condensation of nucleic acid clusters such as reduced cluster volume or cross section, reduced hybridization or clustering time, reduced preparation time, or the like. In some embodiments, a hybridization buffer may comprise one or more of an organic solvent, a buffer, and a polar aprotic solvent.

The organic solvent described herein can have a dielectric constant that is the same as or close to acetonitrile. The dielectric constant of the organic solvent can be in the range of about 20-60, about 25-55, about 25-50, about 25-45, about 25-40, about 30-50, about 30-45, or about 30-40. The dielectric constant of the organic solvent can be greater than 20, 25, 30, 35, or 40. The dielectric constant of the organic solvent can be lower than 30, 40, 45, 50, 55, or 60. The dielectric constant of the organic solvent can be about 35, 36, 37, 38, or 39.

Dielectric constant may be measured using a test capacitator. Representative polar aprotic solvents having a dielectric constant between 30 and 120 may include any such solvent including those disclosed elsewhere herein. Such solvents may particularly include, but are not limited to, acetonitrile, diethylene glycol, N,N-dimethylacetamide, dimethyl formamide, dimethyl sulfoxide, ethylene glycol, formamide, hexamethylphosphoramide, glycerin, methanol, N-methyl-2-pyrrolidinone, nitrobenzene, or nitromethane.

The organic solvent described herein can have a polarity index that is the same as or close to acetonitrile. The polarity index of the organic solvent can be in the range of about 2-9, 2-8,2-7, 2-6, 3-9, 3-8, 3-7, 3-6,4-9, 4-8, 4-7, or 4-6. The polarity index of the organic solvent can be greater than about 2, 3, 4, 4.5, 5, 5.5, or 6. The polarity index of the organic solvent can be lower than about 4.5, 5, 5.5, 6, 6.5, 7, 7.5, 8, 8.5, 9, or 10. The polarity index of the organic solvent can be about 5.5, 5.6, 5.7, or 5.8.

The Snyder Polarity Index may be calculated according to the methods disclosed in Snyder, L. R., Journal of Chromatography A, 92(2):223-30 (1974), which is incorporated by reference herein in it its entirety. Representative polar aprotic solvents having a Snyder polarity index between 6.2 and 7.3 may include any such solvent including those disclosed elsewhere herein. Such solvents may particularly include, but are not limited to, acetonitrile, dimethyl acetamide, dimethyl formamide, N-methyl pyrrolidone, N,N-dimethyl sulfoxide, methanol, or formamide.

Relative polarity may be determined according to the methods given in Reichardt, C., Solvents and Solvent Effects in Organic Chemistry, 3rd ed., 2003, which is incorporated herein by reference in its entirety, and especially with respect to its disclosure of polarities and methods of determining or assessing the same for solvents and solvent molecules. Representative polar aprotic solvents having a relative polarity between 0.44 and 0.82 may include any such solvent as is known in the art or disclosed elsewhere herein. Such solvents may particularly include, but are not limited to, dimethylsulfoxide, acetonitrile, 3-pentanol, 2-pentanol,2-butanol, Cyclohexanol, 1-octanol, 2-propanol, 1-heptanol, butanol, 1-hexanol, 1-pentanol, acetyl acetone, ethyl acetoacetate, 1-butanol, benzyl alcohol, 1-propanol, 2-aminoethanol, Ethanol, diethylene glycol, methanol, ethylene glycol, glycerin, or formamide.

The Solvent Polarity (ET(30)) may be calculated according to the methods disclosed in Reichardt,C., Molecular Interactions, Volume 3, Ratajczak, H. and Orville, W. J., Eds (1982), which is incorporated by reference herein in it its entirety.

Some examples of organic solvent include but are not limited to acetonitrile, dimethylformamide (DMF), dimethylsulfoxide (DMSO), acetanilide, N-acetyl pyrrolidone, 4-amino pyridine, benzamide, benzimidazole, 1,2,3-benzotriazole, butadienedioxide, 2,3-butylene carbonate, γ-butyrolactone, caprolactone (epsilon), chloro maleic anhydride, 2-chlorocyclohexanone, chloroethylene carbonate, chloronitromethane, citraconic anhydride, crotonlactone, 5-cyano-2-thiouracil, cyclopropylnitrile, dimethyl sulfate, dimethyl sulfone, 1,3-dimethyl-5-tetrazole, 1,5-dimethyl tetrazole, 1,2-dinitrobenzene, 2,4-dinitrotoluene, dipheynyl sulfone, 1,2-dinitrobenzene, 2,4-dinitrotoluene, dipheynyl sulfone, epsilon-caprolactam, ethanesulfonylchloride, ethyl ethyl phosphinate, N-ethyl tetrazole, ethylene carbonate, ethylene trithiocarbonate, ethylene glycol sulfate, ethylene glycol sulfite, furfural, 2-furonitrile, 2-imidazole, isatin, isoxazole, malononitrile, 4-methoxy benzonitrile, 1-methoxy-2-nitrobenzene, methyl alpha bromo tetronate, 1-methyl imidazole, N-methyl imidazole, 3-methyl isoxazole, N-methyl morpholine-N-oxide, methyl phenyl sulfone, N-methyl pyrrolidinone, methyl sulfolane, methyl toluenesulfonate, 3-nitroaniline, nitrobenzimidazole, 2-nitrofuran, 1-nitroso-2-pyrolidinone, 2-nitrothiophene, 2-oxazolidinone, 9,10-phenanthrenequinone, N-phenyl sydnone, phthalic anhydride, picolinonitrile (2-cyanopyridine), 1,3-propane sultone, β-propiolactone, propylene carbonate, 4H-pyran-4-thione, 4H-pyran-4-one (γ-pyrone), pyridazine, 2-pyrrolidone, saccharin, succinonitrile, sulfanilamide, sulfolane, 2,2,6,6-tetrachlorocyclohexanone, tetrahydrothiapyran oxide, tetramethylene sulfone (sulfolane), thiazole, 2-thiouracil, 3,3,3-trichloro propene, 1,1,2-trichloro propene, 1,2,3-trichloro propene, trimethylene sulfide-dioxide, and trimethylene sulfite.

Representative polar aprotic solvents having a solvent polarity between 44 and 60 may include any such solvent including those disclosed elsewhere herein. Such solvents may particularly include, but are not limited to, dimethyl sulfoxide, 2-methoxycarbonylphenol, triethyl phosphite, 3-pentanol, acetonitrile, nitromethane, cyclohexanol, 2-pentanol, 4-methyl-1,3, dioxolan-2-one, propylene carbonate, acrylonitrile, 1-phenylethanol, 1-dodecanol, 2-butanol, 2-methylcyclohexanol, 2,6, dimethylphenol, 2,6-xylenol, 1-decanol, cyclopentanol, dimethyl sulfone, 1-octanoldiethylene glycol mono n-butyl ether, butyl digol, 1-heptanol, 3-phenyl-1-propanol, 1,3-dioxolane-2-one, ethylene carbonate, 1-hexanol, 4-chlorobutyronitrile, 5-methyl-2-isopropylphenol, thymol, 3,5,5-trimethyl-1-hexanol, 3-methyl-1-butanol, isoamyl alcohol, 2-methyl-1-propanol, isobutyl alcohol, 2-(tert-butyl)phenol, 1-pentanol, 2-phenylethanol, 2-methylpentane-2,4-diol, dipropylene glycol, 2-isopropylphenol, 2-n-butoxyethanol, ethylene glycol mono-n-butyl ether, 1-butanol, 2-hydroxymethyl-tetrahydrofuran, tetrahydrofurfuryl alcohol, 2-hydroxymethylfuran, furfuryl alcohol, 1-propanol, 2,4-dimethylphenol, 2,4-xylenol, benzyl alcohol, 2-ethoxyphenol, 2-ethoxyethanol, 1,5-pentanediol, 1-bromo-2-propanol, 2-methyl-5-isopropylphenol, carvacrol, 2-aminoethanol, ethanol, n-methylacetamide, 3-chloropropionitrile, 2-propen-1-ol, allyl alcohol, 2-methoxyethanol, 2-methylphenol, o-cresol, 1,3-butanediol, 2-propyn-1-ol, propargyl alcohol, 3-methylphenol, m-cresol, triethylene glycol, diethylene glycol, n-methylformamide, 1,2-propanediol, 1,3-propanediol, 2-chlorophenol, methanol, 1,2-ethanediol, glycol, formamide, 2,2,2-trichloroethanol, 1,2,3-propanetriol, glycerol, 2,2,3,3-tetrafluoro-1-propanol, 2,2,2-trifluoroethanol, 4-n-butylphenol, 4-methylphenol, or p-cresol.

Representative polar aprotic solvents having a dielectric constant in the range of about 30-115 may include any such solvent including those disclosed elsewhere herein. Such solvents may particularly include, but are not limited to, dimethyl sulfoxide, 2-methoxycarbonylphenol, triethyl phosphite, 3-pentanol, acetonitrile, nitromethane, cyclohexanol, 2-pentanol, 4-methyl-1,3, dioxolan-2-one, propylene carbonate, acrylonitrile, 1-phenylethanol, 1-dodecanol, 2-butanol, 2-methylcyclohexanol, 2,6, dimethylphenol, 2,6-xylenol, 1-decanol, cyclopentanol, dimethyl sulfone, 1-octanoldiethylene glycol mono n-butyl ether, butyl digol, 1-heptanol, 3-phenyl-1-propanol, 1,3-dioxolane-2-one, ethylene carbonate, 1-hexanol, 4-chlorobutyronitrile, 5-methyl-2-isopropylphenol, thymol, 3,5,5-trimethyl-1-hexanol, 3-methyl-1-butanol, isoamyl alcohol, 2-methyl-1-propanol, isobutyl alcohol, 2-(tert-butyl)phenol, 1-pentanol, 2-phenylethanol, 2-methylpentane-2,4-diol, dipropylene glycol, 2-isopropylphenol, 2-n-butoxyethanol, ethylene glycol mono-n-butyl ether, 1-butanol, 2-hydroxymethyl-tetrahydrofuran, tetrahydrofurfuryl alcohol, 2-hydroxymethylfuran, furfuryl alcohol, 1-propanol, 2,4-dimethylphenol, 2,4-xylenol, benzyl alcohol, 2-ethoxyphenol, 2-ethoxyethanol, 1,5-pentanediol, 1-bromo-2-propanol, 2-methyl-5-isopropylphenol, carvacrol, 2-aminoethanol, ethanol, n-methylacetamide, 3-chloropropionitrile, 2-propen-1-ol, allyl alcohol, 2-methoxyethanol, 2-methylphenol, o-cresol, 1,3-butanediol, 2-propyn-1-ol, propargyl alcohol, 3-methylphenol, m-cresol, triethylene glycol, diethylene glycol, n-methylformamide, 1,2-propanediol, 1,3-propanediol, 2-chlorophenol, methanol, 1,2-ethanediol, glycol, formamide, 2,2,2-trichloroethanol, 1,2,3-propanetriol, glycerol, 2,2,3,3-tetrafluoro-1-propanol, 2,2,2-trifluoroethanol, 4-n-butylphenol, 4-methylphenol, or p-cresol.

Organic solvent addition: In some instances, the disclosed hybridization buffer formulations may include the addition of an organic solvent. Examples of suitable solvents include, but are not limited to, acetonitrile, ethanol, DMF, and methanol, or any combination thereof at varying percentages (typically >5%). In some instances, the percentage of organic solvent (by volume) included in the hybridization buffer may range from about 1% to about 20%. In some instances, the percentage by volume of organic solvent may be at least 1%, at least 2%, at least 3%, at least 4%, at least 5%, at least 6%, at least 7%, at least 8%, at least 9%, at least 10%, at least 15%, or at least 20%. In some instances, the percentage by volume of organic solvent may be at most 20%, at most 15%, at most 10%, at most 9%, at most 8%, at most 7%, at most 6%, at most 5%, at most 4%, at most 3%, at most 2%, or at most 1%. Any of the lower and upper values described in this paragraph may be combined to form a range included within the present disclosure, for example, the percentage by volume of organic solvent may range from about 4% to about 15%. Those of skill in the art will recognize that the percentage by volume of organic solvent may have any value within this range, e.g., about 7.5%.

When the organic solvent comprises a polar aprotic solvent, the amount of the polar aprotic solvent may be present in an amount effective to denature a double stranded nucleic acid. In some embodiments, the amount of the polar aprotic solvent is greater than about 10% by volume based on the total volume of the formulation. The amount of the polar aprotic solvent is about or more than about 5%, 10%, 15%, 20%, 25%, 30%, 35%, 40%, 50%, 60%, 70%, 80%, 90%, or higher, by volume based on the total volume of the formulation. The amount of the polar aprotic solvent is lower than about 15%, 20%, 25%, 30%, 35%, 40%, 50%, 60%, 70%, 80%, 90%, or higher, by volume based on the total volume of the formulation. In some embodiments, the amount of the polar aprotic solvent is in the range of about 10% to 90% by volume based on the total volume of the formulation. In some embodiments, the amount of the polar aprotic solvent is in the range of about 25% to 75% by volume based on the total volume of the formulation. In some embodiments, the amount of the polar aprotic solvent is in the range of about 10% to 95%, 10% to 85%, 20% to 90%, 20% to 80%, 20% to 75%, or 30% to 60% by volume based on the total volume of the formulation. In some embodiments, the polar aprotic solvent is formamide.

When the organic solvent comprises a polar aprotic solvent, the amount of the aprotic solvent may be present in an amount effective to denature a double stranded nucleic acid. In some embodiments, the amount of the aprotic solvent is greater than about 10% by volume based on the total volume of the formulation. The amount of the aprotic solvent is about or more than about 5%, 10%, 15%, 20%, 25%, 30%, 35%, 40%, 50%, 60%, 70%, 80%, 90%, or higher, by volume based on the total volume of the formulation. The amount of the aprotic solvent is lower than about 15%, 20%, 25%, 30%, 35%, 40%, 50%, 60%, 70%, 80%, 90%, or higher, by volume based on the total volume of the formulation. In some embodiments, the amount of the aprotic solvent is in the range of about 10% to 90% by volume based on the total volume of the formulation. In some embodiments, the amount of the aprotic solvent is in the range of about 25% to 75% by volume based on the total volume of the formulation. In some embodiments, the amount of the aprotic solvent is in the range of about 10% to 95%, 10% to 85%, 20% to 90%, 20% to 80%, 20% to 75%, or 30% to 60% by volume based on the total volume of the formulation.

The composition described herein can include one or more crowding agents enhances molecular crowding. The crowding agent can be selected from the group consisting of polyethylene glycol (PEG), dextran, hydroxypropyl methyl cellulose (HPMC), hydroxyethyl methyl cellulose (HEMC), hydroxybutyl methyl cellulose, hydroxypropyl cellulose, methycellulose, and hydroxyl methyl cellulose, and combination thereof. A preferred crowding agent may comprise one or more of polyethylene glycol (PEG), dextran, proteins, such as ovalbumin or hemoglobin, or Ficoll.

A suitable amount of a crowding agent in the composition allows for, enhances, or facilitates molecular crowding. The amount of the crowding agent is about or more than about 1%, 2%, 3%, 5%, 10%, 15%, 20%, 25%, 30%, 35%, 40%, 50%, 60%, or higher, by volume based on the total volume of the formulation. In some cases, the amount of the molecular crowding agent is greater than 5% by volume based on the total volume of the formulation. The amount of the crowding agent is lower than about 3%, 5%, 10%, 12.5%,15%, 20%, 25%, 30%, 35%, 40%, 50%, 60%, 70%, 80%, 90%, or higher, by volume based on the total volume of the formulation. In some cases, the amount of the molecular crowding agent can be less than 30% by volume based on the total volume of the formulation. In some embodiments, the amount of the organic solvent is in the range of about 25% to 75% by volume based on the total volume of the formulation. In some embodiments, the amount of the organic solvent is in the range of about 1% to 40%, 1% to 35%, 2% to 50%, 2% to 40%, 2% to 35%, 2% to 30%, 2% to 25%, 2% to 20%, 2% to 10%, 5% to 50%, 5% to 40%, 5% to 35%, 5% to 30%, 5% to 25%, 5% to 20%, by volume based on the total volume of the formulation. In some cases, the amount of the molecular crowding agent can be in the range of about 5% to about 20% by volume based on the total volume of the formulation. In some embodiments, the amount of the crowding agent is in the range of about 1% to 30% by volume based on the total volume of the formulation.

One example of the crowding agent in the composition is polyethylene glycol (PEG. In some embodiments, the PEG used can have a molecular weight sufficient to enhance or facilitate molecular crowding. In some embodiments, the PEG used in the composition has a molecular weight in the range of about 5 k-50 k Da. In some embodiments, the PEG used in the composition has a molecular weight in the range of about 10 k-40 k Da. In some embodiments, the PEG used in the composition has a molecular weight in the range of about 10 k-30 k Da. In some embodiments, the PEG used in the composition has a molecular weight in the range of about 20 k Da.

In some instances, the disclosed hybridization buffer formulations may include the addition of a molecular crowding or volume exclusion agent. Molecular crowding or volume exclusion agents are typically macromolecules (e.g., proteins) which, when added to a solution in high concentrations, may alter the properties of other molecules in solution by reducing the volume of solvent available to the other molecules. In some instances, the percentage by volume of molecular crowding or volume exclusion agent included in the hybridization buffer formulation may range from about 1% to about 50%. In some instances, the percentage by volume of molecular crowding or volume exclusion agent may be at least 1%, at least 5%, at least 10%, at least 15%, at least 20%, at least 25%, at least 30%, at least 35%, at least 40%, at least 45%, or at least 50%. In some instances, the percentage by volume of molecular crowding or volume exclusion agent may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of molecular crowding or volume exclusion agent may range from about 5% to about 35%. Those of skill in the art will recognize that the percentage by volume of molecular crowding or volume exclusion agent may have any value within this range, e.g., about 12.5%.

The compositions described herein may include pH buffer system that maintains the pH of the compositions in a range suitable for hybridization process. The pH buffer system can include one or more buffering agents selected from the group consisting of Tris, HEPES, TAPS, Tricine, Bicine, Bis-Tris, NaOH, KOH, TES, EPPS, MES, and MOPS. The pH buffer system can further include a solvent. A preferred pH buffer system includes MOPS, MES, TAPS, phosphate buffer combined with methanol, acetonitrile, ethanol, isopropanol, butanol, t-butyl alcohol, DMF, DMSO, or any combination therein

The amount of the pH buffer system is effective to maintain the pH of the formulation to be in a range suitable for the hybridization. In some instances, the pH may be at least 3, at least 4, at least 5, at least 6, at least 7, at least 8, at least 9, or at least 10. In some instances, the pH may be at most 10, at most 9, at most 8, at most 7, at most 6, at most 5, at most 4, or at most 3. Any of the lower and upper values described in this paragraph may be combined to form a range included within the present disclosure, for example, the pH of the hybridization buffer may range from about 4 to about 8. Those of skill in the art will recognize that the pH of the hybridization buffer may have any value within this range, e.g., about pH 7.8. In some cases, the pH range is about 3 to about 10. In some instances, the disclosed hybridization buffer formulations may include adjustment of pH over the range of about pH 3 to pH 10, with a preferred buffer range of 5-9.

Additives that impact DNA melting temperatures: The compositions described herein can include one or more additives to allow for better control of the melting temperature of the nucleic acid and enhance the stringency control of the hybridization reaction. Hybridization reactions are usually carried out under the stringent conditions in order to achieve hybridization specificity. In some cases, the additive for controlling melting temperature of nucleic acid is formamide.

The amount of the additive for controlling melting temperature of nucleic acid can vary depending on other agents used in the compositions. The amount of the additive for controlling melting temperature of the nucleic acid is about or more than about 1%, 2%, 3%, 5%, 10%, 15%, 20%, 25%, 30%, 35%, 40%, 50%, 60%, or higher, by volume based on the total volume of the formulation. In some cases, the amount of the additive for controlling melting temperature of the nucleic acid is greater than about 2% by volume based on the total volume of the formulation. In some cases, the amount of the additive for controlling melting temperature of the nucleic acid is greater than 5% by volume based on the total volume of the formulation. In some cases, the amount of the additive for controlling melting temperature of the nucleic acid is lower than about 3%, 5%, 10%, 12.5%,15%, 20%, 25%, 30%, 35%, 40%, 50%, 60%, 70%, 80%, 90%, or higher, by volume based on the total volume of the formulation. In some embodiments, the amount of the additive for controlling melting temperature of the nucleic acid is in the range of about 1% to 40%, 1% to 35%, 2% to 50%, 2% to 40%, 2% to 35%, 2% to 30%, 2% to 25%, 2% to 20%, 2% to 10%, 5% to 50%, 5% to 40%, 5% to 35%, 5% to 30%, 5% to 25%, 5% to 20%, by volume based on the total volume of the formulation. In some embodiments, the amount of the additive for controlling melting temperature of the nucleic acid is in the range of about 2% to 20% by volume based on the total volume of the formulation. In some cases, the amount of the additive for controlling melting temperature of the nucleic acid is in the range of about 5% to 10% by volume based on the total volume of the formulation.

In some instances, the disclosed hybridization buffer formulations may include the addition of an additive that alters nucleic acid duplex melting temperature. Examples of suitable additives that may be used to alter nucleic acid melting temperature include, but are not limited to, Formamide. In some instances, the percentage by volume of a melting temperature additive included in the hybridization buffer formulation may range from about 1% to about 50%. In some instances, the percentage by volume of a melting temperature additive may be at least 1%, at least 5%, at least 10%, at least 15%, at least 20%, at least 25%, at least 30%, at least 35%, at least 40%, at least 45%, or at least 50%. In some instances, the percentage by volume of a melting temperature additive may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of a melting temperature additive may range from about 10% to about 25%. Those of skill in the art will recognize that the percentage by volume of a melting temperature additive may have any value within this range, e.g., about 22.5%.

In some instances, the disclosed hybridization buffer formulations may include the addition of an additive that impacts nucleic acid hydration. Examples include, but are not limited to, betaine, urea, glycine betaine, or any combination thereof. In some instances, the percentage by volume of a hydration additive included in the hybridization buffer formulation may range from about 1% to about 50%. In some instances, the percentage by volume of a hydration additive may be at least 1%, at least 5%, at least 10%, at least 15%, at least 20%, at least 25%, at least 30%, at least 35%, at least 40%, at least 45%, or at least 50%. In some instances, the percentage by volume of a hydration additive may be at most 50%, at most 45%, at most 40%, at most 35%, at most 30%, at most 25%, at most 20%, at most 15%, at most 10%, at most 5%, or at most 1%. Any of the lower and upper values described in this paragraph may be combined to form a range included within the present disclosure, for example, the percentage by volume of a hydration additive may range from about 1% to about 30%. Those of skill in the art will recognize that the percentage by volume of a melting temperature additive may have any value within this range, e.g., about 6.5%.

### Low Non-Specific Binding Surfaces

In some embodiments, the methods and compositions disclosed herein may comprise or may further comprise a low non-specific binding surface that enable improved nucleic acid hybridization and amplification performance. In some embodiments, a low nonspecific binding surface may function in part to assist or to support further improvements in clustering performance, such as reduced cluster size, improved clustering efficiency, increased clustering density, etc. in addition to, in concert with, or as an integral part of the role of a low nonspecific binding surface in providing high CNR in images of nucleic acid bound surfaces. In general, the disclosed surface may comprise one or more layers of a covalently or non-covalently attached low-binding, chemical modification layers, e.g., silane layers, polymer films, and one or more covalently or non-covalently attached primer sequences that may be used for tethering single-stranded template oligonucleotides to the surface. In some instances, the formulation of the surface, e.g., the chemical composition of one or more layers, the coupling chemistry used to cross-link the one or more layers to the surface and/or to each other, and the total number of layers, may be varied such that non-specific binding of proteins, nucleic acid molecules, and other hybridization and amplification reaction components to the surface is minimized or reduced relative to a comparable monolayer. Often, the formulation of the surface may be varied such that non-specific hybridization on the surface is minimized or reduced relative to a comparable monolayer. The formulation of the surface may be varied such that non-specific amplification on the surface is minimized or reduced relative to a comparable monolayer. The formulation of the surface may be varied such that specific amplification rates and/or yields on the surface are maximized. Amplification levels suitable for detection are achieved in no more than 2, 3, 4, 5, 6, 7, 8, 9, 10, 15, or 30 amplification cycles in some cases disclosed herein.

Examples of materials from which the substrate or support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The substrate or support structure may be rendered in any of a variety of geometries and dimensions, and may comprise any of a variety of materials. For example, in some instances the substrate or support structure may be locally planar (e.g., comprising a microscope slide or the surface of a microscope slide). Globally, the substrate or support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some instances, the surface of the substrate or support structure used for nucleic acid hybridization and amplification may be a solid, non-porous surface. In some instances, the surface of the substrate or support structure used for nucleic acid hybridization and amplification may be porous, such that the coatings described herein penetrate the porous surface, and nucleic acid hybridization and amplification reactions performed thereon may occur within the pores.

The substrate or support structure that comprises the one or more chemically-modified layers, e.g., layers of a low non-specific binding polymer, may be independent or integrated into another structure or assembly. For example, in some instances, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some preferred embodiments, the substrate or support structure comprises the interior surface (such as the lumen surface) of a capillary. In alternate preferred embodiments the substrate or support structure comprises the interior surface (such as the lumen surface) of a capillary etched into a planar chip.

The chemical modification layers may be applied uniformly across the surface of the substrate or support structure. Alternately, the surface of the substrate or support structure may be non-uniformly distributed or patterned, such that the chemical modification layers are confined to one or more discrete regions of the substrate. For example, the substrate surface may be patterned using photolithographic techniques to create an ordered array or random pattern of chemically-modified regions on the surface. Alternately or in combination, the substrate surface may be patterned using, e.g., contact printing and/or ink-jet printing techniques. In some instances, an ordered array or random patter of chemically-modified discrete regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 50,000, 500,000, 1,000,000 or more discrete regions, or any intermediate number spanned by the range herein.

In order to achieve low nonspecific binding surfaces (also referred to herein as “low binding” or “passivated” surfaces), hydrophilic polymers may be nonspecifically adsorbed or covalently grafted to the substrate or support surface. Typically, passivation is performed utilizing poly(ethylene glycol) (PEG, also known as polyethylene oxide (PEO) or polyoxyethylene), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, dextran, or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some instances, two or more layers of a hydrophilic polymer, e.g., a linear polymer, branched polymer, or multi-branched polymer, may be deposited on the surface. In some instances, two or more layers may be covalently coupled to each other or internally cross-linked to improve the stability of the resulting surface. In some instances, oligonucleotide primers with different base sequences and base modifications (or other biomolecules, e.g., enzymes or antibodies) may be tethered to the resulting surface layer at various surface densities. In some instances, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

As a result of the surface passivation techniques disclosed herein, proteins, nucleic acids, and other biomolecules do not “stick” to the substrates, that is, they exhibit low nonspecific binding (NSB). Examples are shown below using standard monolayer surface preparations with varying glass preparation conditions. Hydrophilic surface that have been passivated to achieve ultra-low NSB for proteins and nucleic acids require novel reaction conditions to improve primer deposition reaction efficiencies, hybridization performance, and induce effective amplification. All of these processes require oligonucleotide attachment and subsequent protein binding and delivery to a low binding surface. As described below, the combination of a new primer surface conjugation formulation (Cy3 oligonucleotide graft titration) and resulting ultra-low non-specific background (NSB functional tests performed using red and green fluorescent dyes) yielded results that demonstrate the viability of the disclosed approaches. Some surfaces disclosed herein exhibit a ratio of specific (e.g., hybridization to a tethered primer or probe) to nonspecific binding (e.g., Binter) of a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein. Some surfaces disclosed herein exhibit a ratio of specific to nonspecific fluorescence signal (e.g., for specifically-hybridized to nonspecifically bound labeled oligonucleotides, or for specifically-amplified to nonspecifically-bound (Binter) or non-specifically amplified (Bintra) labeled oligonucleotides or a combination thereof (Binter+Bintra)) for a fluorophore such as Cy3 of at least 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 50:1, 75:1, 100:1, or greater than 100:1, or any intermediate value spanned by the range herein.

In order to scale primer surface density and potentially to add additional dimensionality to hydrophilic or amphoteric surfaces, substrates comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some instances, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some instances, high primer density materials may be constructed in solution and subsequently layered onto the surface in multiple steps.

The attachment chemistry used to graft a first chemically-modified layer to a support surface will generally be dependent on both the material from which the support is fabricated and the chemical nature of the layer. In some instances, the first layer may be covalently attached to the support surface. In some instances, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques may be used to clean or treat the support surface. For example, glass or silicon surfaces may be acid-washed using a Piranha solution (a mixture of sulfuric acid (H2SO4) and hydrogen peroxide (H₂O₂)) and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding support surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules including, but not limited to, amino acids, peptides, nucleotides, oligonucleotides, other monomers or polymers, or combinations thereof may be used in creating the one or more chemically-modified layers on the support surface, where the choice of components used may be varied to alter one or more properties of the support surface, e.g., the surface density of functional groups and/or tethered oligonucleotide primers, the hydrophilicity/hydrophobicity of the support surface, or the three three-dimensional nature (i.e., “thickness”) of the support surface. Examples of preferred polymers that may be used to create one or more layers of low non-specific binding material in any of the disclosed support surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the support surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

One or more layers of a multi-layered surface may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branced PHEMA), branched poly(oligo(ethylene glycol) methyl ether methacrylate) (branched POEGMA), branched polyglutamic acid (branched PGA), branched poly-lysine, branched poly-glucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches. Molecules often exhibit a ‘power of 2’ number of branches, such as 2, 4, 8, 16, 32, 64, or 128 branches.

PEG multilayers include PEG (8,16,8) on PEGamine-APTES, exposed to two layers of 7 uM primer pre-loading, exhibited a concentration of 2,000,000 to 10,000,000 on the surface. Similar concentrations were observed for 3-layer multi-arm PEG (8,16,8) and (8,64,8) on PEGamine-APTES exposed to 8 uM primer, and 3-layer multi-arm PEG (8,8,8) using star-shape PEG-amine to replace dumbbell-shaped 16mer and 64mer. PEG multilayers having comparable first, second and third PEG level are also contemplated.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some instances, e.g., wherein at least one layer of a multi-layered surface comprises a branched polymer, the number of covalent bonds between a branched polymer molecule of the layer being deposited and molecules of the previous layer may range from about one covalent linkages per molecule and about 32 covalent linkages per molecule. In some instances, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 or more than 32 covalent linkages per molecule.

Any reactive functional groups that remain following the coupling of a material layer to the support surface may be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface of the disclosed low binding supports may range from 1 to about 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials. In some instances at least one layer may comprise a branched polymer. In some instance, all of the layers may comprise a branched polymer.

One or more layers of low non-specific binding material may in some cases be deposited on and/or conjugated to the substrate surface using a polar protic solvent, a polar aprotic solvent, a nonpolar solvent, or any combination thereof. In some instances the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some instances, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of water or an aqueous buffer solution. In some instances, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, or any percentage spanned or adjacent to the range herein, with the balance made up of an organic solvent. The pH of the solvent mixture used may be less than 5, 5, 5, 5, 6, 6, 6.5, 7, 7.5, 8, 8.5, 9, 9.5, 10, or greater than 10, or any value spanned or adjacent to the range described herein.

In some instances, one or more layers of low non-specific binding material may be deposited on and/or conjugated to the substrate surface using a mixture of organic solvents, wherein the dielectric constant of at least once component is less than 40 and constitutes at least 50% of the total mixture by volume. In some instances, the dielectric constant of the at least one component may be less than 10, less than 20, less than 30, less than 40. In some instances, the at least one component constitutes at least 20%, at least 30%, at least 40%, at least 50%, at least 50%, at least 60%, at least 70%, or at least 80% of the total mixture by volume.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some instances, exposure of the surface to fluorescent dyes (e.g., Cy3, Cy5, etc.), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some instances, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some instances, other techniques, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some instances, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some instances, the label may comprise a fluorescent label. In some instances, the label may comprise a radioisotope. In some instances, the label may comprise any other detectable. In some instances, the degree of non-specific binding exhibited by a given support surface formulation may thus be assessed in terms of the number of non-specifically bound protein molecules (or other molecules) per unit area. In some instances, the low-binding supports of the present disclosure may exhibit non-specific protein binding (or non-specific binding of other specified molecules, e.g., Cy3 dye) of less than 0.001 molecule per μm2, less than 0.01 molecule per μm2, less than 0.1 molecule per μm2, less than 0.25 molecule per μm2, less than 0.5 molecule per μm2, less than 1 molecule per μm2, less than 10 molecules per μm2, less than 100 molecules per μm2, or less than 1,000 molecules per μm2. Those of skill in the art will realize that a given support surface of the present disclosure may exhibit non-specific binding falling anywhere within this range, for example, of less than 86 molecules per μm2. For example, some modified surfaces disclosed herein exhibit nonspecific protein binding of less than 0.5 molecule/μm2 following contact with a 1 uM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm2. In independent nonspecific binding assays, 1 uM labeled Cy3 SA (ThermoFisher), 1 uM Cy5 SA dye (ThermoFisher), 10 uM Aminoallyl-dUTP—ATTO-647N (Jena Biosciences), 10 uM Aminoallyl-dUTP—ATTO-Rho11 (Jena Biosciences), 10 uM Aminoallyl-dUTP—ATTO-Rho 11 (Jena Biosciences), 10 uM 7-Propargylamino-7-deaza-dGTP—Cy5 (Jena Biosciences, and 10 uM 7-Propargylamino-7-deaza-dGTP—Cy3 (Jena Biosciences) were incubated on the low binding substrates at 37° C. for 15 minutes in a 384 well plate format. Each well was rinsed 2-3× with 50 ul deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm2.

In some instances, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some instances, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some instances, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding support surfaced disclosed herein may range from about 0 degrees to about 30 degrees. In some instances, the water contact angle for the hydrophilic, low-binding support surfaced disclosed herein may no more than 50 degrees, 45 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some instances, the hydrophilic surfaces disclosed herein facilitate reduced wash times for bioassays, often due to reduced nonspecific binding of biomolecules to the low-binding surfaces. In some instances, adequate wash steps may be performed in less than 60, 50, 40, 30, 20, 15, 10, or less than 10 seconds. For example, in some instances adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, in some instances, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some instances, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some instances, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some instances, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

Fluorescence excitation energies vary among particular fluorophores and protocols, and may range in excitation wavelength from less than 400 nm to over 800 nm, consistent with fluorophore selection or other parameters of use of a surface disclosed herein. Accordingly, low background surfaces as disclosed herein exhibit low background fluorescence signals or high contrast to noise (CNR) ratios relative to other surfaces. For example, in some instances, the background fluorescence of the surface at a location that is spatially distinct or removed from a labeled feature on the surface (e.g., a labeled spot, cluster, discrete region, sub-section, or subset of the surface) comprising a hybridized cluster of nucleic acid molecules, or a clonally-amplified cluster of nucleic acid molecules produced by 20 cycles of nucleic acid amplification via thermocycling, may be no more than 20×, 10×, 5×, 2×, 1×, 0.5×, 0.1×, or less than 0.1× greater than the background fluorescence measured at that same location prior to performing said hybridization or said 20 cycles of nucleic acid amplification.

In some instances, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

The surface that comprises the one or more chemically-modified layers, e.g., layers of a low non-specific binding polymer, may be independent or integrated into another structure or assembly. The chemical modification layers may be applied uniformly across the surface. Alternately, the surface may be patterned, such that the chemical modification layers are confined to one or more discrete regions of the substrate. For example, the surface may be patterned using photolithographic techniques to create an ordered array or random pattern of chemically-modified regions on the surface. Alternately or in combination, the substrate surface may be patterned using, e.g., contact printing and/or ink-jet printing techniques. In some instances, an ordered array or random patter of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces (also referred to herein as “low binding” or “passivated” surfaces), hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some instances, two or more layers of a hydrophilic polymer, e.g., a linear polymer, branched polymer, or multi-branched polymer, may be deposited on the surface. In some instances, two or more layers may be covalently coupled to each other or internally cross-linked to improve the stability of the resulting surface. In some instances, oligonucleotide primers with different base sequences and base modifications (or other biomolecules, e.g., enzymes or antibodies) may be tethered to the resulting surface layer at various surface densities. In some instances, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some instances, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some instances, high primer density materials may be constructed in solution and subsequently layered onto the surface in multiple steps.

The attachment chemistry used to graft a first chemically-modified layer to a surface will generally be dependent on both the material from which the surface is fabricated and the chemical nature of the layer. In some instances, the first layer may be covalently attached to the surface. In some instances, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques may be used to clean or treat the surface. For example, glass or silicon surfaces may be acid-washed using a Piranha solution (a mixture of sulfuric acid (H2SO4) and hydrogen peroxide (H₂O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of preferred polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

One or more layers of a multi-layered surface may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some instances, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some instances, e.g., wherein at least one layer of a multi-layered surface comprises a branched polymer, the number of covalent bonds between a branched polymer molecule of the layer being deposited and molecules of the previous layer may range from about one covalent linkages per molecule and about 32 covalent linkages per molecule. In some instances, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some instances, the number of layers is at least 1, at least 2, at least 3, at least 4, at least 5, at least 6, at least 7, at least 8, at least 9, or at least 10. In some instances, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some instances the number of layers may range from about 2 to about 4. In some instances, all of the layers may comprise the same material. In some instances, each layer may comprise a different material. In some instances, the plurality of layers may comprise a plurality of materials. In some instances at least one layer may comprise a branched polymer. In some instance, all of the layers may comprise a branched polymer.

One or more layers of low non-specific binding material may in some cases be deposited on and/or conjugated to the substrate surface using a polar protic solvent, an organic solvent, a nonpolar solvent, or any combination thereof. In some instances the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some instances, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some instances, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than 9mk.

As noted, the low non-specific binding surface exhibit reduced non-specific binding of nucleic acids, and other components of the hybridization and/or amplification formulation used for solid-phase nucleic acid amplification. The degree of non-specific binding exhibited by a given surface may be assessed either qualitatively or quantitatively. For example, in some instances, exposure of the surface to fluorescent dyes (e.g., Cy3, Cy5, etc.), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding surface comprising different surface formulations. In some instances, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on surfaces comprising different surface formulations—provided that care has been taken to ensure that the fluorescence imaging is performed under conditions where fluorescence signal is linearly related (or related in a predictable manner) to the number of fluorophores on the surface (e.g., under conditions where signal saturation and/or self-quenching of the fluorophore is not an issue) and suitable calibration standards are used. In some instances, other techniques, for example, radioisotope labeling and counting methods may be used for quantitative assessment of the degree to which non-specific binding is exhibited by the different surface formulations of the present disclosure.

As noted, in some instances, the degree of non-specific binding exhibited by the disclosed low-binding surfaces may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some instances, the label may comprise a fluorescent label. In some instances, the label may comprise a radioisotope. In some instances, the label may comprise any other detectable label. In some instances, the degree of non-specific binding exhibited by a given surface formulation may thus be assessed in terms of the number of non-specifically bound protein molecules (or other molecules) per unit area. In some instances, the low-binding surfaces of the present disclosure may exhibit non-specific protein binding (or non-specific binding of other specified molecules, e.g., Cy3 dye) of less than 0.001 molecule per μm2, less than 0.01 molecule per μm2, less than 0.1 molecule per μm2, less than 0.25 molecule per μm2, less than 0.5 molecule per μm2, less than 1 molecule per μm2, less than 10 molecules per μm2, less than 100 molecules per μm2, or less than 1,000 molecules per μm2. Those of skill in the art will realize that a given surface of the present disclosure may exhibit non-specific binding falling anywhere within this range, for example, of less than 86 molecules per μm2. For example, some modified surfaces disclosed herein exhibit nonspecific protein binding of less than 0.5 molecule/μm2 following contact with a 1 μM solution of bovine serum albumin (BSA) in phosphate buffered saline (PBS) buffer for 30 minutes, followed by a 10 minute PBS rinse. Some modified surfaces disclosed herein exhibit nonspecific binding of Cy3 dye molecules of less than 0.25 molecules per μm2.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some instances, the degree of hydrophilicity (or “wettability” with aqueous solutions) of the disclosed surfaces may be assessed, for example, through the measurement of water contact angles in which a small droplet of water is placed on the surface and its angle of contact with the surface is measured using, e.g., an optical tensiometer. In some instances, a static contact angle may be determined. In some instances, an advancing or receding contact angle may be determined. In some instances, the water contact angle for the hydrophilic, low-binding surfaces disclosed herein may range from about 0 degrees to about 30 degrees. In some instances, the water contact angle for the hydrophilic, low-binding surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding surface of the present disclosure may exhibit a water contact angle having a value of anywhere within this range.

In some instances, the low-binding surfaces of the present disclosure may exhibit significant improvement in stability or durability to prolonged exposure to solvents and elevated temperatures, or to repeated cycles of solvent exposure or changes in temperature. For example, in some instances, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some instances, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some instances, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some instances, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface. Accordingly, low background surfaces as disclosed herein exhibit low background fluorescence signals or high contrast to noise (CNR) ratios relative to other surfaces.

In general, at least one layer of the one or more layers of low non-specific binding material may comprise functional groups for covalently or non-covalently attaching oligonucleotide adapter or primer sequences, or the at least one layer may already comprise covalently or non-covalently attached oligonucleotide adapter or primer sequences at the time that it is deposited on the support surface. In some instances, the oligonucleotides tethered to the polymer molecules of at least one layer may be distributed at a plurality of depths throughout the layer.

One or more types of oligonucleotide primer may be attached or tethered to the support surface. In some instances, the one or more types of oligonucleotide adapters or primers may comprise spacer sequences, adapter sequences for hybridization to adapter-ligated template library nucleic acid sequences, forward amplification primers, reverse amplification primers, sequencing primers, and/or molecular barcoding sequences, or any combination thereof. In some instances, 1 primer or adapter sequence may be tethered to at least one layer of the surface. In some instances, at least 2, 3, 4, 5, 6, 7, 8, 9, 10, or more than 10 different primer or adapter sequences may be tethered to at least one layer of the surface.

In some instances, the tethered oligonucleotide adapter and/or primer sequences may range in length from about 10 nucleotides to about 100 nucleotides. In some instances, the tethered oligonucleotide adapter and/or primer sequences may be at least 10, at least 20, at least 30, at least 40, at least 50, at least 60, at least 70, at least 80, at least 90, or at least 100 nucleotides in length. In some instances, the tethered oligonucleotide adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some instances the length of the tethered oligonucleotide adapter and/or primer sequences may range from about 20 nucleotides to about 80 nucleotides. Those of skill in the art will recognize that the length of the tethered oligonucleotide adapter and/or primer sequences may have any value within this range, e.g., about 24 nucleotides.

In some instances, the tethered primer sequences may comprise modifications designed to facilitate the specificity and efficiency of nucleic acid amplification as performed on low-binding supports. For example, in some instances the primer may comprise polymerase stop points such that the stretch of primer sequence between the surface conjugation point and the modification site is always in single-stranded form and functions as a loading site for 5′ to 3′ helicases in some helicase-dependent isothermal amplification methods. Other examples of primer modifications that may be used to create polymerase stop points include, but are not limited to, an insertion of a PEG chain into the backbone of the primer between two nucleotides towards the 5′ end, insertion of an abasic nucleotide (i.e., a nucleotide that has neither a purine nor a pyrimidine base), or a lesion site which can be bypassed by the helicase.

In some embodiments, it may be desirable to vary the surface density of tethered primers on the support surface and/or the spacing of the tethered primers away from the support surface (e.g., by varying the length of a linker molecule used to tether the primers to the surface) in order to “tune” the support for optimal performance when using a given amplification method. As noted below, adjusting the surface density of tethered primers may impact the level of specific and/or non-specific amplification observed on the support in a manner that varies according to the amplification method selected. In some instances, the surface density of tethered oligonucleotide primers may be varied by adjusting the ratio of molecular components used to create the support surface. For example, in the case that an oligonucleotide primer— PEG conjugate is used to create the final layer of a low-binding support, the ratio of the oligonucleotide primer— PEG conjugate to a non-conjugated PEG molecule may be varied. The resulting surface density of tethered primer molecules may then be estimated or measured using any of a variety of techniques. Examples include, but are not limited to, the use of radioisotope labeling and counting methods, covalent coupling of a cleavable molecule that comprises an optically-detectable tag (e.g., a fluorescent tag) that may be cleaved from a support surface of defined area, collected in a fixed volume of an appropriate solvent, and then quantified by comparison of fluorescence signals to that for a calibration solution of known optical tag concentration, or using fluorescence imaging techniques provided that care has been taken with the labeling reaction conditions and image acquisition settings to ensure that the fluorescence signals are linearly related to the number of fluorophores on the surface (e.g., that there is no significant self-quenching of the fluorophores on the surface).

In some instances, the resultant surface density of oligonucleotide primers on the low binding support surfaces of the present disclosure may range from about 1,000 primer molecules per μm2 to about 100,000 primer molecules per μm2. In some instances, the surface density of oligonucleotide primers may be at least 1,000, at least 10,000, or at least 100,000, molecules per μm2. In some instances, the surface density of oligonucleotide primers may be at most 500,000, at most 100,000, at most 10,000, at most 1,000, or at most 100 molecules per μm2. Any of the lower and upper values described in this paragraph may be combined to form a range included within the present disclosure, for example, in some instances the surface density of primers may range from about 1,000 molecules per μm2 to about 10,000 molecules per μm2. Those of skill in the art will recognize that the surface density of primer molecules may have any value within this range, e.g., about 4,000 or about 5,000 molecules per μm2. In some instances, the surface density of template library nucleic acid sequences initially hybridized to adapter or primer sequences on the support surface may be less than or equal to that indicated for the surface density of tethered oligonucleotide primers. In some instances, the surface density of clonally-amplified template library nucleic acid sequences hybridized to adapter or primer sequences on the support surface may span the same range as that indicated for the surface density of tethered oligonucleotide primers. In some instances, the surface density of clonally-amplified template library nucleic acid sequences hybridized to adapter or primer sequences on the support surface may be less than that indicated for the surface density of tethered oligonucleotide primers.

Local densities as listed above do not preclude variation in density across a surface, such that a surface may comprise a region having an oligo density of, for example, 500, 5,000, 50,000/μm2, or more, while also comprising at least a second region having a substantially different local density.

In some instances, the use of the buffer formulations disclosed herein (in some embodiments, used in combination with low non-specific binding surface) yield relative hybridization rates that range from about 2× to about 20× faster than that for a conventional hybridization protocol. In some instances, the relative hybridization rate may be at least 2×, at least 3×, at least 4×, at least 5×, at least 6×, at least 7×, at least 8×, at least 9×, at least 10×, at least 12×, at least 14×, at least 16×, at least 18×, or at least 20× that for a conventional hybridization protocol.

The method and composition described herein can help shorten the time required for completing the hybridization step. In some embodiments, the hybridization time can be in the range of about is to 2 h, about 5 s to 1.5 h, about 15 s to 1 h, or about 15 s to 0.5 h. In some embodiments, the hybridization time can be in the range of about 15 s to 1 h. In some embodiments, the hybridization time can be shorter than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, the hybridization time can be longer than 1 s, 5 s, 10 s, 15 s, 30 s, 1 min, 1.5 min, 2 min, 2.5 min, 3 min, 4 min, or 5 min.

The annealing methods described herein can significantly shorten the annealing time. In some embodiments, at least 90% of the target nucleic acid anneals to the surface bound nucleic acid in less than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, at least 80% of the target nucleic acid anneals to the surface bound nucleic acid in less than 15 s, 30 s, 1 min, 1.5 min, 2 min, 2.5 min, 3 min, 4 min, 5 min, 6 min, 7 min, 8 min, 9 min, 10 min, 15 min, 20 min, 25 min, 30 min, 40 min, 50 min, 60 min, 70 min, 80 min, 90 min, 100 min, 110 min, or 120 min. In some embodiments, at least 90% of the target nucleic acid anneals to the surface bound nucleic acid in greater than 1 s, 5 s, 10 s, 15 s, 30 s, 1 min, 1.5 min, 2 min, 2.5 min, 3 min, 4 min, or 5 min. In some embodiments, at least 90% of the target nucleic acid anneals to the surface bound nucleic acid in the range of about 10 s to about 1 hour, about 30 s to about 50 min, about 1 min to about 50 min, or about 1 min to about 30 min.

Improvements in hybridization efficiency: As used herein, hybridization efficiency (or yield) is a measure of the percentage of total available surface-tethered adapter sequences, nontethered adapter sequences, condenser sequences, primer sequences, oligonucleotide sequences, or other sequences that are hybridized to complementary sequences. In some instances, the use of optimized buffer formulations disclosed herein (in some embodiments, used in combination with low non-specific binding surface) yield improved hybridization efficiency compared to that for a conventional hybridization protocol. In some instances, the hybridization efficiency that may be achieved is better than 80%, 85%, 90%, 95%, 98%, or 99% in any of the hybridization reaction times specified above.

The methods and compositions described herein can be used in an isothermal annealing conditions. In some embodiments, one or more of the methods described herein can eliminate the cooling step required for most hybridization steps. In some embodiments, the annealing methods described herein can be performed at a temperature in the range of about 10° C. to 95° C., about 20° C. to 80° C., about 30° C. to 70° C. In some embodiments, the temperature can be lower than about 40° C., 50° C., 60° C., 70° C., 80° C., or 90° C.

As used herein, hybridization specificity is a measure of the ability of tethered adapter sequences, primer sequences, or oligonucleotide sequences in general to correctly hybridize only to completely complementary sequences. In some instances, the use of the optimized buffer formulations disclosed herein (in some embodiments, used in combination with low non-specific binding surface) yield improved hybridization specificity compared to that for a conventional hybridization protocol. In some instances, the hybridization specificity that may be achieved is better than 1 base mismatch in 10 hybridization events, 1 base mismatch in 100 hybridization events, 1 base mismatch in 1,000 hybridization events, or 1 base mismatch in 10,000 hybridization events.

### Nucleic Acid Sequencing

Provided herein, in some embodiments, are methods, systems, and kits for performing nucleic acid sequencing of circularized nucleic acid libraries. In some embodiments, sequencing comprises sequential addition of labeled nucleotides to a growing nucleic acid in the 5′ to 3′ direction using an enzyme, where the growing nucleic acid is complementary to a target nucleic acid immobilized on a surface. In some embodiments, the labeled nucleotides may be labeled with a fluorescent label, biotin, other labels described herein, or any combinations thereof. As the growing nucleic acid sequentially incorporates labeled nucleotides, the label may be detected, for instance, through fluorescence imaging so that the base identity of the nucleotide is determined. In some embodiments, the enzyme is a polymerase, a ligase, or another enzyme disclosed herein.

In one example method, base-calling signal strength is significantly improved by combining some of the methods disclosed herein. In this method, a target nucleic acid is circularized and immobilized onto a surface. In some embodiments, the target nucleic acid is immobilized by hybridization to a surface-bound primer, which is attached to the surface by suitable means disclosed here (e.g., silane chemistries). In the case of on-surface circularization, in some embodiments, the surface-bound primer may be the splint nucleic acid molecule designed to fascilitate circularization of a linear target nucleic acid in the presence of a lligating enzyme described herein. In the case of in-solution cirularization, in some embodments, the circularized target nucleic acid is bound to the surface by hybridization to a surface-bound primer containing a nucleic acid sequence compelentary to an index sequence present in the circularized target nucleic acid (introduced using the methods descried herein). In some embodiments, rolling circle amplification is carried out using the circularized nucleic acid as a template to create amplicons comprising multiple copies of the circular nucleic acid template on the surface. In some embodiment, the copies are concatemers comprising multiple copies of an identical sequence (the target nucleic acid sequence). In some embodiments, those amplicons (as referred to here, in this context, as “derivatives” of the target nucleic acid) are linear. In some embodiments, a primer sequence is hybridized to the circularized nucleic acid or derivatives thereof to form primed nucleic acid templates for the sequencing reaction. Sequencing (e.g. base calling) starts by introducing polymerase and a labeled nucleotide or nucleotide moiety to the primed templates, where the polymerase recognizes the primer of the primed template and reversibly binds with the primed templates. In some embodiments, the nucleotide is labeled directly (e.g., such as at the base of the nucleotide). In some embodiments, the nucleotide is not labeled. In some embodiments, the nucleotide moiety is conjugated to a polymer core that is labeled (e.g., nucleotide-polymer conjugate). In some embodiments, the label is irradiated to produce a signal that is optically detected. In some embodiments, the labeled nucleotide or nucleotide moiety is washed away, and unlabeled nucleotide dNTP is introduced to the system. In some embodiments, the primed template is blocked, thereby preventing incorporation of the labeled nucleotide or nucleotide moiety. In such embodiments, a deblocking step is performed after the labeled nucleotide or nucleotide moiety is washed away to permit incorporation of the unlabeled nucleotide. The primed template and the nucleotide dNTP bind together near an active site of the polymerase, the polymerase catalyzes a reaction which adds the nucleotide dNTP to the growing strand that is complementary to the primed template, ending one round of the base calling procedure. In some embodiments, the dNTP is modified with a blocking group at its 3′ position of its sugar. In some embodiments, the blocking group comprises a 3′-O-azido group, a 3′-O-azidomethyl group, a 3′-O-alkyl hydroxylamino group, a 3′-phosphorothioate group, a 3′-O-malonyl group, a 3′-O-benzyl group, or a 3′-O-amino group or derivatives thereof. By repeating the base calling procedure, the entire sequence of the circular nucleic acid or derivative thereof may be determined.

In some embodiments, the order between the circularization, immobilization, and amplification may be performed in any order, such as: circularization, immobilization, then amplification; circularization, amplification, then immobilization; immobilization, circularization, then amplification; immobilization, amplification, then circularization; amplification, circularization, then immobilization; or amplification, immobilization, then circularization.

In some embodiments, adapters or primers may be incorporated into the sequence of the target nucleic acid as an additional step in the method, in a non-limiting example, such as: immobilization, adaptor or primer incorporation, circularization, then amplification. In some embodiments, adapters or primers may be incorporated into the sequence of the target nucleic acid as an additional step in the method, in a non-limiting example, such as: immobilization, circularization, adaptor or primer incorporation, then amplification.

### Paired End Sequencing

In some embodiments, paired-end sequencing allows sequencing of both ends of a nucleic acid molecule by sequencing, from 5′ to 3′ both strands (sense and antisense) of the target double-stranded nucleic acid molecule, which improves sequencing accuracy. The forward and reverse strands of the target double-stranded nucleic acid molecule may be sequenced at the same time, thereby reducing the speed of the sequencing reaction by half as compared to conventional sequencing techniques that sequence the forward and reverse strands sequentially. This is made possible by spatially separating the forward and reverse strands on an array or surface that are known. In this manner, a corresponding reverse nucleic acid molecule in known proximity from the forward strand may be identified as such as sequenced simultaneously.

Disclosed herein are methods and systems for paired-end sequencing of circular nucleic acid molecules containing both the forward and the reverse strands of a target double-stranded nucleic acid molecule. A library of target nucleic acid molecules may be generating using methods described herein. In some embodiments, the circular nucleic acid molecule is a single sequencing template comprising the forward and reverse strands and that may include sites for primer attachment allowing simultaneous sequencing (either by using the same or different primers) or sequential sequencing (such as by using different primers for the forward and reverse strands).

### Detection Methods

In some embodiments, sequencing methods utilizing the compositions and methods disclosed herein may incorporate a detection method enabling base calling to reveal the sequence of the target nucleic acid. In some embodiments, these detection methods may include any method for nucleic acid detection and/or nucleic acid sequencing. In some embodiments, the systems described herein are used to perform the base calling procedure. In some embodiments, said detection methods may include, for example, one or more of fluorescence detection, colorimetric detection, luminescence (such as chemiluminescence of bioluminescence) detection, interferometric detection, resonance-based detection such as Raman detection, spin resonance-based detection, NMR-based detection, and the like, and other methods such as electrical detection, such as, for example, capacitance-based detection, impedance based detection, or electrochemical detection, such as detection of electrons generated by or within a chemical reaction, or combinations of electrical, such as, e.g., impedance measurements, with other, e.g., optical measurements.

### Nucleotide Binding Reaction

In some embodiments, whether paired-end sequencing or otherwise, the nucleic acid sequencing is performed using a nucleotide binding reaction that precludes incorporation of the detectable nucleotide into the primed template (e.g., primed circular nucleic acid molecule). In some embodiments, the detectable nucleotide comprises a label coupled thereto directly or indirectly.

In some embodiments, the detectable nucleotide may comprise a blocking group that inhibits the activity of an enzyme that would otherwise incorporate the nucleotide into a growing nucleic acid change. In some embodiments, nucleotides with a blocking group may comprise a nucleotide that has been modified to contain a blocking group at the 3′ position; a nucleotide that has been modified with a 3′-O-azido group, a 3′-O-azidomethyl group, a 3′-O-alkyl hydroxylamino group, a 3′-phosphorothioate group, a 3′-O-malonyl group, a 3′-O-benzyl group, or a 3′-O-amino group or derivatives thereof. In some embodiments, the detectable nucleotide may lack certain groups, when the groups would otherwise allow incorporation the nucleotide into a growing nucleic acid chain. In some embodiments, a nucleotide lacking a 3′ hydroxyl is inhibited from being incorporated into a growing nucleic acid chain.

In some embodiments, the detectable nucleotide moiety is conjugated to a polymer core, also known as a polymer-nucleotide conjugate. In some embodiments, polymers include linear or branched polyethylene glycol (PEG), linear or branched polypropylene glycol, linear or branched polyvinyl alcohol, linear or branched polylactic acid, linear or branched polyglycolic acid, linear or branched polyglycine, linear or branched polyvinyl acetate, a dextran, or other such polymers, or copolymers incorporating any two or more of the foregoing or incorporating other polymers as are known in the art. In one embodiment, the polymer is a PEG. In another embodiment, the polymer can have PEG branches.

Suitable polymers may be characterized by a repeating unit incorporating a functional group suitable for derivatization such as an amine, a hydroxyl, a carbonyl, or an allyl group. The polymer can also have one or more pre-derivatized substituents such that one or more particular subunits will incorporate a site of derivatization or a branch site, whether or not other subunits incorporate the same site, substituent, or moiety. A pre-derivatized substituent may comprise or may further comprise, for example, a nucleotide, a nucleoside, a nucleotide analog, a label such as a fluorescent label, radioactive label, or spin label, an interaction moiety, an additional polymer moiety, or the like, or any combination of the foregoing.

In the polymer-nucleotide conjugate, the polymer can have a plurality of branches. The branched polymer can have various configurations, including but are not limited to stellate (“starburst”) forms, aggregated stellate (“hater skelter”) forms, bottle brush, or dendrimer. The branched polymer can radiate from a central attachment point or central moiety, or may incorporate multiple branch points, such as, for example, 2, 3, 4, 5, 6, 7, 8, 9, 10, or more branch points. In some embodiments, each subunit of a polymer may optionally constitute a separate branch point.

The length and size of the branch can differ based on the type of polymer. In some branched polymers, the branch may have a length of between 1 and 1,000 nm, between 1 and 100 nm, between 1 and 200 nm, between 1 and 300 nm, between 1 and 400 nm, between 1 and 500 nm, between 1 and 600 nm, between 1 and 700 nm, between 1 and 800 nm, or between 1 and 900 nm, or more, or having a length falling within or between any of the values disclosed herein.

In some polymer-nucleotide conjugates, the polymer core may have a size corresponding to an apparent molecular weight of 1K Da, 2K Da, 3K Da, 4K Da, 5K Da, 10K Da, 15K Da, 20K Da, 30K Da, 50K Da, 80K Da, 100K Da, or any value within a range defined by any two of the foregoing. The apparent molecular weight of a polymer may be calculated from the known molecular weight of a representative number of subunits, as determined by size exclusion chromatography, as determined by mass spectrometry, or as determined by any other method as is known in the art.

In some branched polymers, the branch may have a size corresponding to an apparent molecular weight of 1K Da, 2K Da, 3K Da, 4K Da, 5K Da, 10K Da, 15K Da, 20K Da, 30K Da, 50K Da, 80K Da, 100K Da, or any value within a range defined by any two of the foregoing. The apparent molecular weight of a polymer may be calculated from the known molecular weight of a representative number of subunits, as determined by size exclusion chromatography, as determined by mass spectrometry, or as determined by any other method as is known in the art. The polymer can have multiple branches. The number of branches in the polymer can be 2, 3, 4, 5, 6, 7, 8, 12, 16, 24, 32, 64, 128 or more, or a number falling within a range defined by any two of these values.

For polymer-nucleotide conjugates comprising a branched polymer of, for example, a branched PEG comprising 4, 8, 16, 32, or 64 branches, the polymer nucleotide conjugate can have nucleotides attached to the ends of the PEG branches, such that each end has attached thereto 0, 1, 2, 3, 4, 5, 6 or more nucleotides. In one non-limiting example, a branched PEG polymer of between 3 and 128 PEG arms may have attached to the ends of the polymer branches one or more nucleotides, such that each end has attached thereto 0, 1, 2, 3, 4, 5, 6 or more nucleotides or nucleotide analogs. In some embodiments, a branched polymer or dendrimer has an even number of arms. In some embodiments, a branched polymer or dendrimer has an odd number of arms.

In some instances, the length of the linker (e.g., a PEG linker) may range from about 1 nm to about 1,000 nm. In some instances, the length of the linker may be at least 1 nm, at least 10 nm, at least 25 nm, at least 50 nm, at least 75 nm, at least 100 nm, at least 200 nm, at least 300 nm, at least 400 nm, at least 500 nm, at least 600 nm, at least 700 nm, at least 800 nm, at least 900 nm, or at least 1,000 nm. In some instances, the length of the linker may range between any two of the values in this paragraph. For example, in some instances, the length of the linker may range from about 75 nm to about 400 nm. Those of skill in the art will recognize that in some instances, the length of the linker may have any value within the range of values in this paragraph, e.g., 834 nm.

In some instances, the length of the linker is different for different nucleotides (including deoxyribonucleotides and ribonucleotides), nucleotide analogs (including deoxyribonucleotide analogs and ribonucleotide analogs), nucleosides (including deoxyribonucleosides or ribonucleosides), or nucleoside analogs (including deoxyribonucleoside analogs or ribonucleoside analogs). In some instances, one of the nucleotides, nucleotide analogs, nucleosides, or nucleoside analogs comprises, for example, deoxyadenosine, and the length of the linker is between 1 nm and 1,000 nm. In some instances, one of the nucleotides, nucleotide analogs, nucleosides, or nucleoside analogs comprises, for example, deoxyguanosine, and the length of the linker is between 1 nm and 1,000 nm. In some instances, one of the nucleotides, nucleotide analogs, nucleosides, or nucleoside analogs comprises, for example, thymidine, and the length of the linker is between 1 nm and 1,000 nm. In some instances, one of the nucleotides, nucleotide analogs, nucleosides, or nucleoside analogs comprises, for example, comprises deoxyuridine, and the length of the linker is between 1 nm and 1,000 nm. In some instances, one of the nucleotides, nucleotide analogs, nucleosides, or nucleoside analogs comprises, for example, deoxycytidine, and the length of the linker is between 1 nm and 1,000 nm. In some instances, one of the nucleotides, nucleotide analogs, nucleosides, or nucleoside analogs comprises, for example, adenosine, and the length of the linker is between 1 nm and 1,000 nm. In some instances, one of the nucleotides, nucleotide analogs, nucleosides, or nucleoside analogs comprises, for example, guanosine, and the length of the linker is between 1 and 1,000 nm. In some instances, one of the nucleotides, nucleotide analogs, nucleosides, or nucleoside analogs comprises, for example, 5-methyl-uridine, and the length of the linker is between 1 nm and 1,000 nm. In some instances, one of the nucleotides, nucleotide analogs, nucleosides, or nucleoside analogs comprises, for example, uridine, and the length of the linker is between 1 nm and 1,000 nm. In some instances, one of the nucleotides, nucleotide analogs, nucleosides, or nucleoside analogs comprises, for example, cytidine, and the length of the linker is between 1 nm and 1,000 nm.

In the polymer-nucleotide conjugate, each branch or a subset of branches of the polymer may have attached thereto a moiety comprising a nucleotide moiety (e.g., comprising an adenine, a thymine, a uracil, a cytosine, or a guanine residue or a derivative or mimetic thereof). In some embodiment, the nucleotide moiety is capable of binding or incorporation to a polymerase, reverse transcriptase, or other nucleotide binding or incorporation domain. Optionally, the nucleotide moiety may be capable of being incorporated into an elongating nucleic acid chain during a polymerase reaction, such as a primed template during a sequencing reaction disclosed herein. In some instances, said nucleotide moiety may be blocked such that it is not capable of being incorporated into an elongating nucleic acid chain during a polymerase reaction. In some other instances, said moiety may be reversibly blocked such that it is not capable of being incorporated into an elongating nucleic acid chain during a polymerase reaction until such block is removed, after which said moiety is then capable of being incorporated into an elongating nucleic acid chain during a polymerase reaction. By way of example, the nucleotide moiety may include a 3′ deoxyribonucleotide, a 3′ azidonucleotide, a 3′-methyl azido nucleotide, or another such nucleotide, so as to not be capable of being incorporated into an elongating nucleic acid chain during a polymerase reaction. In some embodiments, the nucleotide moiety can include a 3′-O-azido group, a 3′-O-azidomethyl group, a 3′-phosphorothioate group, a 3′-O-malonyl group, a 3′-O-alkyl hydroxylamino group, or a 3′-O-benzyl group. In some embodiments, the nucleotide lacks a 3′ hydroxyl group. The nucleotide can be conjugated to the polymer branch through the 5′ end of the nucleotide moiety. A non-limiting example of a polymer-nucleotide conjugate is provided in FIG. 28.

The polymer can further have a binding or incorporation moiety in each branch or a subset of branches. Some examples of the binding or incorporation moiety include but are not limited to biotin, avidin, strepavidin or the like, polyhistidine domains, complementary paired nucleic acid domains, G-quartet forming nucleic acid domains, calmodulin, maltose-binding protein, cellulase, maltose, sucrose, glutathione-S-transferase, glutathione, O-6-methylguanine-DNA methyltransferase, benzylguanine and derivatives thereof, benzylcysteine and derivatives thereof, an antibody, an epitope, a protein A, a protein G. The binding or incorporation moiety can be any interactive molecules or fragment thereof known in the art to bind to or facilitate interactions between proteins, between proteins and ligands, between proteins and nucleic acids, between nucleic acids, or between small molecule interaction domains or moieties.

In some embodiments, a composition as provided herein may comprise one or more elements of a complementary interaction moiety. Non-limiting examples of complementary interaction moieties include, for example, biotin and avidin; SNAP-benzylguanosine; antibody or FAB and epitope; IgG FC and Protein A, Protein G, ProteinA/G, or Protein L; maltose binding protein and maltose; lectin and cognate polysaccharide; ion chelation moieties, complementary nucleic acids, nucleic acids capable of forming triplex or triple helical interactions; nucleic acids capable of forming G-quartets, and the like. One of skill in the art will readily recognize that many pairs of moieties exist and are commonly used for their property of interacting strongly and specifically with one another; and thus any such complementary pair or set is considered to be suitable for this purpose in constructing or envisioning the compositions of the present disclosure. In some embodiments, a composition as disclosed herein may comprise compositions in which one element of a complementary interaction moiety is attached to one molecule or multivalent ligand, and the other element of the complementary interaction moiety is attached to a separate molecule or multivalent ligand. In some embodiments, a composition as disclosed herein may comprise compositions in which both or all elements of a complementary interaction moiety are attached to a single molecule or multivalent ligand. In some embodiments, a composition as disclosed herein may comprise compositions in which both or all elements of a complementary interaction moiety are attached to separate arms of, or locations on, a single molecule or multivalent ligand. In some embodiments, a composition as disclosed herein may comprise compositions in which both or all elements of a complementary interaction moiety are attached to the same arm of, or locations on, a single molecule or multivalent ligand. In some embodiments, compositions comprising one element of a complementary interaction moiety and compositions comprising another element of a complementary interaction moiety may be simultaneously or sequentially mixed. In some embodiments, interactions between molecules or particles as disclosed herein allow for the association or aggregation of multiple molecules or particles such that, for example, detectable signals are increased. In some embodiments, fluorescent, colorimetric, or radioactive signals are enhanced. In other embodiments, other interaction moieties as disclosed herein or as are known in the art are contemplated. In some embodiments, a composition as provided herein may be provided such that one or more molecules comprising a first interaction moiety such as, for example, one or more imidazole or pyridine moieties, and one or more additional molecules comprising a second interaction moiety such as, for example, histidine residues, are simultaneously or sequentially mixed. In some embodiments, said composition comprises 1, 2, 3, 4, 5, 6, or more imidazole or pyridine moieties. In some embodiments, said composition comprises 1, 2, 3, 4, 5, 6, or more histidine residues. In such embodiments, interaction between the molecules or particles as provided may be facilitated by the presence of a divalent cation such as nickel, manganese, magnesium, calcium, strontium, or the like. In some embodiments, for example, a (His)3 group may interact with a (His)3 group on another molecule or particle via coordination of a nickel or manganese ion.

The multivalent binding or incorporation composition may comprise one or more buffers, salts, ions, or additives. In some embodiments, representative additives may include, but are not limited to, betaine, spermidine, detergents such as Triton X-100, Tween 20, SDS, or NP-40, ethylene glycol, polyethylene glycol, dextran, polyvinyl alcohol, vinyl alcohol, methylcellulose, heparin, heparan sulfate, glycerol, sucrose, 1,2-propanediol, DMSO, N,N,N-trimethylglycine, ethanol, ethoxyethanol, propylene glycol, polypropylene glycol, block copolymers such as the Pluronic (r) series polymers, arginine, histidine, imidazole, or any combination thereof, or any substance known in the art as a DNA “relaxer” (a compound, with the effect of altering the persistence length of DNA, altering the number of within-polymer junctions or crossings, or altering the conformational dynamics of a DNA molecule such that the accessibility of sites within the strand to DNA binding or incorporation moieties is increased).

The multivalent binding or incorporation composition may include zwitterionic compounds as additives. Further representative additives may be found in Lorenz, T. C. J. Vis. Exp. (63), e3998, doi:10.3791/3998 (2012), which is hereby incorporated by reference with respect to its disclosure of additives for the facilitation of nucleic acid binding or dynamics, or the facilitation of processes involving the manipulation, use, or storage of nucleic acids. In some embodiments, representative cations may include, but are not limited to, sodium, magnesium, strontium, potassium, manganese, calcium, lithium, nickel, cobalt, or other such cations as are known in the art to facilitate nucleic acid interactions, such as self-association, secondary or tertiary structure formation, base pairing, surface association, peptide association, protein binding, or the like.

When the multivalent binding or incorporation composition is used in replacement of single unconjugated or untethered nucleotide to form a complex with the polymerase and one or more copies of the target nucleic acid, the local concentration of the nucleotide as well as the binding avidity of the complex (in the case that a complex comprising two or more target nucleic acid molecules is formed) is increased many-fold, which in turn enhances the signal intensity, particularly the correct signal versus mismatch. The present disclosure contemplates contacting the multivalent binding or incorporation composition with a polymerase and a primed target nucleic acid to determine the formation of a ternary binding or incorporation complex.

In various embodiments, polymerases suitable for the binding or incorporation interaction describe herein include may include any polymerase as is or may be known in the art. It is, for example, known that every organism encodes within its genome one or more DNA polymerases. Examples of suitable polymerases may include but are not limited to: Klenow DNA polymerase, Thermus aquaticus DNA polymerase I (Taq polymerase), KlenTaq polymerase, and bacteriophage T7 DNA polymerase; human alpha, delta and epsilon DNA polymerases; bacteriophage polymerases such as T4, RB69 and phi29 bacteriophage DNA polymerases, Pyrococcus furiosus DNA polymerase (Pfu polymerase); Bacillus subtilis DNA polymerase III, and E. coli DNA polymerase III alpha and epsilon; 9 degree N polymerase, reverse transcriptases such as HIV type M or 0 reverse transcriptases, avian myeloblastosis virus reverse transcriptase, or Moloney Murine Leukemia Virus (MMLV) reverse transcriptase, or telomerase. Further non-limiting examples of DNA polymerases can include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as Vent™, Deep Vent™, Pfu, KOD, Pfx, Therminator™, and Tgo polymerases. In some embodiments, the polymerase is a klenow polymerase.

The present disclosure contemplates contacting the multivalent binding or incorporation composition comprising at least one particle-nucleotide conjugate with one or more polymerases. In some embodiments, the contacting is done in the presence of one or more target nucleic acids. In some embodiments, the target nucleic acids are primed circular nucleic acids or derivatives thereof. In some embodiments, the target nucleic acids are single stranded nucleic acids. In some embodiments, the target nucleic acids are primed single stranded nucleic acids. In some embodiments, the target nucleic acids are double stranded nucleic acids. In some embodiments, the contacting comprises contacting the multivalent binding or incorporation composition with one polymerase. In some embodiments, the contacting comprises the contacting of the composition comprising one or more nucleotides with multiple polymerases. The polymerase can be bound to a single nucleic acid molecule.

The binding between target nucleic acid and multivalent binding composition may be provided in the presence of a polymerase that has been rendered catalytically inactive. In one embodiment, the polymerase may have been rendered catalytically inactive by mutation. In one embodiment, the polymerase may have been rendered catalytically inactive by chemical modification. In some embodiments, the polymerase may have been rendered catalytically inactive by the absence of a necessary substrate, ion, or cofactor. In some embodiments, the polymerase enzyme may have been rendered catalytically inactive by the absence of magnesium ions.

The binding between target nucleic acid and multivalent binding composition occur in the presence of a polymerase wherein the binding solution, reaction solution, or buffer lacks magnesium or manganese. Alternatively, the binding between target nucleic acid and multivalent binding composition occur in the presence of a polymerase wherein the binding solution, reaction solution, or buffer comprises calcium or strontium.

When the catalytically inactive polymerases are used to help a nucleic acid interact with a multivalent binding composition, the interaction between said composition and said polymerase stabilizes a ternary complex so as to render the complex detectable by fluorescence or by other methods as disclosed herein or otherwise known in the art. Unbound polymer-nucleotide conjugates may optionally be washed away prior to detection of the ternary binding complex.

Contacting of one or more nucleic acids with the polymer-nucleotide conjugates disclosed herein in a solution containing either one of calcium or magnesium or containing both calcium and magnesium. Alternatively, the contacting of one or more nucleic acids with the polymer-nucleotide conjugates disclosed herein in a solution lacking either one of calcium or magnesium, or lacking both calcium or magnesium, and in a separate step, without regard to the order of the steps, adding to the solution one of calcium or magnesium, or both calcium and magnesium. In some embodiments, the contacting of one or more nucleic acids with the polymer-nucleotide conjugates disclosed herein in a solution lacking strontium, and comprises in a separate step, without regard to the order of the steps, adding to the solution strontium.

### Systems

Disclosed herein are systems for caring out the methods of the present disclosure, including preparing a nucleic acid library (e.g., a circular library and/or sequencing the library using one or more components of the system. In some embodiments, the system comprises one or more computer processors individually or collectively programmed to implement a method comprising: (a) bringing a nucleic acid sequence into contact with said surface under conditions sufficient to couple said nucleic acid sequence or derivative thereof to said surface; (b) enzymatically circularizing said nucleic acid sequence or a derivative thereof to produce a circular nucleic acid sequence; (c) contacting said circular nucleic acid sequence or derivative thereof with a primer sequence complementary thereto, thereby producing a primed nucleic acid sequence; and/or (d) performing a nucleotide binding reaction with said primed nucleic acid sequence or a derivative thereof to identify a nucleotide of said primed nucleic acid sequence or derivative thereof. Systems can also include a surface described herein. In some embodiments, the surface is an interior surface of a flow cell. In some embodiments, the surface comprises a plurality of immobilized nucleic acids coupled thereto. In some embodiments, the system further comprises polymer-nucleotide conjugate disclosed herein. In some embodiments, the system further comprises unlabeled nucleotides having a blocking group at a 3′ position of a sugar of the unlabeled nucleotide. In some embodiments, the immobilized nucleic acids are primed. In some embodiments, the immobilized nucleic acids are circular. In some embodiments, the immobilized nucleic acids are primed. In some embodiments, the immobilized nucleic acids have been amplified using rolling circle amplification to produce derivatives of the immobilized nucleic acids. In some embodiments, the immobilized nucleic acids or derivatives thereof are primed by with a primer sequence that is complementary to at least a portion of the sequence of the immobilized nucleic acids or derivatives thereof. In some embodiments, the polymer-nucleotide conjugate includes a polymer core and a plurality of nucleotide moieties attached to the polymer core. In some embodiments, each polymer-nucleotide conjugate includes a detection moiety (e.g., a detectable label) coupled thereto. In some embodiments, the nucleotide moiety includes a detection moiety. In some embodiments, the nucleotide moiety includes a moiety that blocks the incorporation of the moiety into an elongating nucleic acid molecule. In some embodiments, the moiety comprises a 3′-O-azido group, a 3′-O-azidomethyl group, a 3′-O-alkyl hydroxylamino group, a 3′-phosphorothioate group, a 3′-O-malonyl group, a 3′-O-benzyl group, or a 3′-O-amino group or derivatives thereof.

In some embodiments, systems comprise reagents or compositions disclosed herein in a fluid. For example, the system may comprise a fluid comprising a synthetic ligating enzyme or enzymatically-active fragment thereof, a synthetic splint nucleic acid molecule, or a combination thereof. In some embodiments, the system comprises a fluid comprising one or more nucleotides or nucleotide-polymer conjugates, a polymerizing enzyme, or a combination thereof. In some embodiments, the systems comprise a kit described herein with such components as well as instructions for how to use the kit to prepare the circular nucleic acid sequencing libraries described herein, and optionally, how to sequence them according to the methods described herein.

The systems may also comprise a fluidics module configured to bring the reagents and components of the system into contact with said surface. In some embodiments, the systems comprise an imaging module comprising one or more light sources, one or more optical components, and one or more image sensors operably connected to the surface for performing the nucleic acid sequencing reaction (e.g., nucleotide binding reaction, sequencing by incorporation, etc.).

Flow Cells

Disclosed herein are flow cells that include a first reservoir housing a first solution and having an inlet end and an outlet end, wherein the first agent flows from the inlet end to the outlet end in the first reservoir; a second reservoir housing a second solution and having an inlet end and an outlet end, wherein the second agent flows from the inlet end to the outlet end in the second reservoir; a central region having an inlet end fluidically coupled to the outlet end of the first reservoir and the outlet end of the second reservoir through at least one valve. In the flow cell device, the volume of the first solution flowing from the outlet of the first reservoir to the inlet of the central region is less than the volume of the second solution flowing from the outlet of the second reservoir to the inlet of the central region.

The reservoirs described in the device can be used to house different reagents. In some aspects, the first solution housed in the first reservoir is different from the second solution that is housed in the second reservoir. The second solution comprises at least one reagent common to a plurality of reactions occurring in the central region. In some aspects, the second solution comprises at least one reagent selected from the list consisting of a solvent, a polymerase, and a dNTP. In some aspects, the second solution comprise low cost reagents. In some aspects, the first reservoir is fluidically coupled to the central region through a first valve and the second reservoir is fluidically coupled to the central region through a second valve. The valve can be a diaphragm valve or other suitable valves.

The design of the flow cell device can achieve a more efficient use of the reaction reagents than other sequencing device, particularly for costly reagents used in a variety of sequencing steps. In some aspects, the first solution comprises a reagent and the second solution comprises a reagent and the reagent in the first solution is more expensive than the reagent in the second solution. In some aspects, the first solution comprises a reaction-specific reagent and the second solution comprises nonspecific reagent common to all reaction occurring in the central region, and wherein the reaction specific reagent is more expensive than the nonspecific reagent. In some aspects, the first reservoir is positioned in close proximity to the inlet of the central region to reduce dead volume for delivery of the first solutions. In some aspects, the first reservoir is places closer to the inlet of the central region than the second reservoir. In some aspects, the reaction-specific reagent is configured in close proximity to the second diaphragm valve so as to reduce dead volume relative to delivery of the plurality of nonspecific reagents from the plurality of reservoirs to the first diaphragm valve.

(a) Central Region

The central region can include a capillary tube or microfluidic chip having one or more microfluidic channels. In some embodiments, the capillary tube is an off-shelf product. The capillary tube or the microfluidic chip can also be removable from the device. In some embodiments, the capillary tube or microfluidic channel comprises an oligonucleotide population directed to sequence a eukaryotic genome. In some embodiments, the capillary tube or microfluidic channel in the central region can be removable.

Capillary Flow Cell Devices

Disclosed herein are single capillary flow cell devices that comprise a single capillary and one or two fluidic adapters affixed to one or both ends of the capillary, where the capillary provides a fluid flow channel of specified cross-sectional area and length, and where the fluidic adapters are configured to mate with standard tubing to provide for convenient, interchangeable fluid connections with an external fluid flow control system.

FIG. 24A illustrates one non-limiting example of a single glass capillary flow cell device that comprises two fluidic adaptors 2401— one affixed to each end of the piece of glass capillary—that are designed to mate with standard OD fluidic tubing. In some instances, the flow cell does not comprise fluidic tubing. The fluidic adaptors can be attached to the capillary using any of a variety of techniques known to those of skill in the art including, but not limited to, press fit, adhesive bonding, solvent bonding, laser welding, etc., or any combination thereof. In some embodiments, the capillary used in the disclosed flow cell devices (and flow cell cartridges to be described below) will have at least one internal, axially-aligned fluid flow channel (or “lumen”) 2402 that runs the full length of the capillary. In some aspects, the capillary may have two, three, four, five, or more than five internal, axially-aligned fluid flow channels (or “lumen”).

A number specified cross-sectional geometries for a single capillary (or lumen thereof) are consistent with the disclosure herein, including, but not limited to, circular, elliptical, square, rectangular, triangular, rounded square, rounded rectangular, or rounded triangular cross-sectional geometries. In some aspects, the single capillary (or lumen thereof) may have any specified cross-sectional dimension or set of dimensions. For example, in some aspects the largest cross-sectional dimension of the capillary lumen (e.g. the diameter if the lumen is circular in shape or the diagonal if the lumen is square or rectangular in shape) may range from about 10 μm to about 10 mm. In some aspects, the largest cross-sectional dimension of the capillary lumen may be at least 10 μm, at least 25 μm, at least 50 μm, at least 75 μm, at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In some aspects, the largest cross-sectional dimension of the capillary lumen may be at most 10 mm, at most 9 mm, at most 8 mm, at most 7 mm, at most 6 mm, at most 5 mm, at most 4 mm, at most 3 mm, at most 2 mm, at most 1 mm, at most 900 μm, at most 800 μm, at most 700 μm, at most 600 μm, at most 500 μm, at most 400 μm, at most 300 μm, at most 200 μm, at most 100 μm, at most 75 μm, at most 50 μm, at most 25 μm, or at most 10 μm. Any of the lower and upper values described in this paragraph may be combined to form a range included within the present disclosure, for example, in some aspects the largest cross-sectional dimension of the capillary lumen may range from about 100 μm to about 500 μm. Those of skill in the art will recognize that the largest cross-sectional dimension of the capillary lumen may have any value within this range, e.g., about 124 μm.

The length of the one or more capillaries used to fabricate the disclosed single capillary flow cell devices or flow cell cartridges may range from about 5 mm to about 5 cm or greater. In some instances, the length of the one or more capillaries may be less than 5 mm, at least 5 mm, at least 1 cm, at least 1.5 cm, at least 2 cm, at least 2.5 cm, at least 3 cm, at least 3.5 cm, at least 4 cm, at least 4.5 cm, or at least 5 cm. In some instances, the length of the one or more capillaries may be at most 5 cm, at most 4.5 cm, at most 4 cm, at most 3.5 cm, at most 3 cm, at most 2.5 cm, at most 2 cm, at most 1.5 cm, at most 1 cm, or at most 5 mm. Any of the lower and upper values described in this paragraph may be combined to form a range included within the present disclosure, for example, in some instances the length of the one or more capillaries may range from about 1.5 cm to about 2.5 cm. Those of skill in the art will recognize that the length of the one or more capillaries may have any value within this range, e.g., about 1.85 cm. In some instances, devices or cartridges may comprise a plurality of two or more capillaries that are the same length. In some instances, devices or cartridges may comprise a plurality of two or more capillaries that are of different lengths.

Capillaries in some cases have a gap height of about or exactly 50, 75, 100, 125, 150, 175, 200, 225, 250, 275, 300, 350, 400, or 500 μm, or any value falling within the range defined thereby. Some preferred embodiments have gap heights of about 50 μm-200 μm, 50 μm to 150 μm, or comparable gap heights. The capillaries used for constructing the disclosed single capillary flow cell devices or capillary flow cell cartridges may be fabricated from any of a variety of materials known to those of skill in the art including, but not limited to, glass (e.g., borosilicate glass, soda lime glass, etc.), fused silica (quartz), polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives. PEI is somewhere between polycarbonate and PEEK in terms of both cost and compatibility. FFKM is also known as Kalrez or any combination thereof.

The capillaries used for constructing the disclosed single capillary flow cell devices or capillary flow cell cartridges may be fabricated using any of a variety of techniques known to those of skill in the art, where the choice of fabrication technique is often dependent on the choice of material used, and vice versa. Examples of suitable capillary fabrication techniques include, but are not limited to, extrusion, drawing, precision computer numerical control (CNC) machining and boring, laser photoablation, and the like. Devices can be pour molded or injection molded to fabricate any three dimensional structure for adapting to single piece flow cell.

Examples of commercial vendors that provide precision capillary tubing include Accu-Glass (St. Louis, Mo.; precision glass capillary tubing), Polymicro Technologies (Phoenix, Ariz.; precision glass and fused-silica capillary tubing), Friedrich & Dimmock, Inc. (Millville, N.J.; custom precision glass capillary tubing), and Drummond Scientific (Broomall, Pa.; OEM glass and plastic capillary tubing).

Microfluidic Chip Flow Cell Devices

Disclosed herein also include flow cell devices that comprise one or more microfluidic chips and one or two fluidic adapters affixed to one or both ends of the microfluidic chips, where the microfluidic chip provides one or more fluid flow channels of specified cross-sectional area and length, and where the fluidic adapters are configured to mate with the microfluidic chip to provide for convenient, interchangeable fluid connections with an external fluid flow control system.

A non-limiting example of a microfluidic chip flow cell device that comprises two fluidic adaptors—one affixed to each end of the microfluidic chip (e.g., the inlet of the microfluidic channels). The fluidic adaptors can be attached to the chip or channel using any of a variety of techniques known to those of skill in the art including, but not limited to, press fit, adhesive bonding, solvent bonding, laser welding, etc., or any combination thereof. In some instances, the inlet and/or outlet of the microfluidic channels on the chip are apertures on the top surface of the chip, and the fluidic adaptors can be attached or coupled to the inlet and outlet of the microfluidic chips.

When the central region comprises a microfluidic chip, the chip microfluidic chip used in the disclosed flow cell deices will have at least a single layer having one or more channels. In some aspects, the microfluidic chip has two layers bonded together to form one or more channels. In some aspects, the microfluidic chip can include three layers bonded together to form one or more channels. In some embodiments, the microfluidic channel has an open top. In some embodiments, the microfluidic channel is positioned between a top layer and a bottom layer.

In general, the microfluidic chip used in the disclosed flow cell devices (and flow cell cartridges to be described below) will have at least one internal, axially-aligned fluid flow channel (or “lumen”) that runs the full length or a partial length of the chip. In some aspects, the microfluidic chip may have two, three, four, five, or more than five internal, axially-aligned microfluidic channels (or “lumen”). The microfluidic channel can be divided into a plurality of frames.

A number specified cross-sectional geometries for a single channels are consistent with the disclosure herein, including, but not limited to, circular, elliptical, square, rectangular, triangular, rounded square, rounded rectangular, or rounded triangular cross-sectional geometries. In some aspects, the channel may have any specified cross-sectional dimension or set of dimensions.

The microfluidic chip used for constructing the disclosed flow cell devices or flow cell cartridges may be fabricated from any of a variety of materials known to those of skill in the art including, but not limited to, glass (e.g., borosilicate glass, soda lime glass, etc.), quartz, polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives. In some embodiments, the microfluidic chip comprises quartz. In some embodiments, the microfluidic chip comprises borosilicate glass.

The microfluidic chips used for constructing the described flow cell devices or flow cell cartridges may be fabricated using any of a variety of techniques known to those of skill in the art, where the choice of fabrication technique is often dependent on the choice of material used, and vice versa. The microfluidic channels on the chip can be constructed using techniques suitable for forming micro-structure or micro-pattern on the surface. In some aspects, the channel is formed by laser irradiation. In some aspects, the microfluidic channel is formed by focused femtosecond laser radiation. In some aspects, the microfluidic channel is formed by etching, including but not limited to chemical or laser etching.

When the microfluidic channels are formed on the microfluidic chip through etching, the microfluidic chip will comprise at least one etched layer. In some aspects, the microfluidic chip can include comprise one non-etched layer, and one non-etched layer, with the etched layer being bonded to the non-etched layer such that the non-etched layer forms a bottom layer or a cover layer for the channels. In some aspects, the microfluidic chip can include comprise one non-etched layer, and two non-etched layers, and wherein the etched layer is positioned between the two non-etched layers.

The chip described herein includes one or more microfluidic channels etched on the surface of the chip. The microfluidic channels are defined as fluid conduits with at least one minimum dimension from <1 nm to 1000 μm. The microfluidic channels can be fabricated through several different methods, such as laser radiation (e.g., femtosecond laser radiation), lithography, chemical etching, and any other suitable methods. Channels on the chip surface can be created by selective patterning and plasma or chemical etching. The channels can be open, or they can be sealed by a conformal deposited film or layer on top to create subsurface or buried channels in the chip. In some embodiments, the channels are created from the removal of a sacrificial layer on the chip. This method does not require the bulk wafer to be etched away. Instead, the channel is located on the surface of the wafer. Examples of direct lithography include electron beam direct-write and focused ion beam milling.

The microfluidic channel system is coupled with an imaging system to capture or detect signals of DNA bases. The microfluidic channel system, fabricated on either a glass or silicon substrate, has channel heights and widths on the order of <1 nm to 1000 μm. For example, in some embodiments a channel may have a depth of 1-50 μm, 1-100 μm, 1-150 μm, 1-200 μm, 1-250 μm, 1-300 μm, 50-100 μm, 50-200 μm, or 50-300 μm, or greater than 300 μm, or a range defined by any two of these values. In some embodiments, a channel may have a depth of 3 mm or more. In some embodiments, a channel may have a depth of 30 mm or more. In some embodiments, a channel may have a length of less than 0.1 mm, between 0.1 mm and 0.5 mm, between 0.1 mm and 1 mm, between 0.1 mm and 5 mm, between 0.1 mm and 10 mm, between 0.1 mm and 25 mm, between 0.1 mm and 50 mm, between 0.1 mm and 100 mm, between 0.1 mm and 150 mm, between 0.1 mm and 200 mm, between 0.1 mm and 250 mm, between 1 mm and 5 mm, between 1 mm and 10 mm, between 1 mm and 25 mm, between 1 mm and 50 mm, between 1 mm and 100 mm, between 1 mm and 150 mm, between 1 mm and 200 mm, between 1 mm and 250 mm, between 5 mm and 10 mm, between 5 mm and 25 mm, between 5 mm and 50 mm, between 5 mm and 100 mm, between 5 mm and 150 mm, between 5 mm and 200 mm, between 1 mm and 250 mm, or greater than 250 mm, or a range defined by any two of these values. In some embodiments, a channel may have a length of 2 m or more. In some embodiments, a channel may have a length of 20 m or more. In some embodiments, a channel may have a width of less than 0.1 mm, between 0.1 mm and 0.5 mm, between 0.1 mm and 1 mm, between 0.1 mm and 5 mm, between 0.1 mm and 10 mm, between 0.1 mm and 15 mm, between 0.1 mm and 20 mm, between 0.1 mm and 25 mm, between 0.1 mm and 30 mm, between 0.1 mm and 50 mm, or greater than 50 mm, or a range defined by any two of these values. In some embodiments, a channel may have a width of 500 mm or more. In some embodiments, a channel may have a width of 5 m or more. The channel length can be in the micrometer range.

The one or more materials used to fabricate the capillaries or microfluidic chips for the disclosed devices are often optically transparent to facilitate use with spectroscopic or imaging-based detection techniques. The entire capillary will be optically transparent. Alternately, only a portion of the capillary (e.g., an optically transparent “window”) will be optically transparent. In some instances, the entire microfluidic chip will be optically transparent. In some instances, only a portion of the microfluidic chip (e.g., an optically transparent “window”) will be optically transparent.

As noted above, the fluidic adapters that are attached to the capillaries or microfluidic channels of the flow cell devices and cartridges disclosed herein are designed to mate with standard OD polymer or glass fluidic tubing or microfluidic channel. As illustrated in FIG. 1, one end of the fluidic adapter may be designed to mate to capillary having specific dimensions and cross-sectional geometry, while the other end may be designed to mate with fluidic tubing having the same or different dimensions and cross-sectional geometry. The adapters may be fabricated using any of a variety of suitable techniques (e.g., extrusion molding, injection molding, compression molding, precision CNC machining, etc.) and materials (e.g., glass, fused-silica, ceramic, metal, polydimethylsiloxane, polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), etc.), where the choice of fabrication technique is often dependent on the choice of material used, and vice versa.

An interior surface (or surface of a capillary lumen) of one or more capillaries or the channel on the microfluidic chip is often coated using any of a variety of surface modification techniques or polymer coatings described herein.

(b) Capillary Flow Cell Cartridges

Also disclosed herein are capillary flow cell cartridges that may comprise one, two, or more capillaries to create independent flow channels. FIG. 24B provides a non-limiting example of capillary flow cell cartridge that comprises two glass capillaries, fluidic adaptors (two per capillary in this example) 2401, and a cartridge chassis 2403 that mates with the capillaries and/or fluidic adapters 2401 such that the capillaries are held in a fixed orientation relative to the cartridge. In some instances, the fluidic adaptors may be integrated with the cartridge chassis. In some instances, the cartridge may comprise additional adapters that mate with the capillaries and/or capillary fluidic adapters. In some instances, the capillaries are permanently mounted in the cartridge. In some instances, the cartridge chassis is designed to allow one or more capillaries of the flow cell cartridge to be interchangeable removed and replaced. For example, in some instances, the cartridge chassis may comprise a hinged “clamshell” configuration which allows it to be opened so that one or more capillaries may be removed and replaces. In some instances, the cartridge chassis is configured to mount on, for example, the stage of a microscope system or within a cartridge holder of an instrument system. In some embodiments, the flow cell comprises openings 2404 that permit heat exchange or cooling during use.

The capillary flow cell cartridges of the present disclosure may comprise a single capillary. In some instances, the capillary flow cell cartridges of the present disclosure may comprise 2, 3, 4, 5, 6, 7, 8, 9, 10, 11, 12, 13, 14, 15, 16, 17, 18, 19, 20, or more than 20 capillaries. The one or more capillaries of the flow cell cartridge may have any of the geometries, dimensions, material compositions, and/or coatings as described above for the single capillary flow cell devices. Similarly, the fluidic adapters for the individual capillaries in the cartridge (typically two fluidic adapters per capillary) may have any of the geometries, dimensions, and material compositions as described above for the single capillary flow cell devices, except that in some instances the fluidic adapters may be integrated directly with the cartridge chassis as illustrated in FIG. 24B. In some instances, the cartridge may comprise additional adapters (i.e., in addition to the fluidic adapters) that mate with the capillaries and/or fluidic adapters and help to position the capillaries within the cartridge. These adapters may be constructed using the same fabrication techniques and materials as those outlined above for the fluidic adapters.

In some embodiments, one or more devices according to the present disclosure may comprise a first surface in an orientation generally facing the interior of the flow channel, wherein said surface may further comprise a polymer coating as disclosed elsewhere herein, and wherein said surface may further comprise one or more oligonucleotides such as a capture oligonucleotide, an adapter oligonucleotide, or any other oligonucleotide as disclosed herein. In some embodiments, said devices may further comprise a second surface in an orientation generally facing the interior of the flow channel and further generally facing or parallel to the first surface, wherein said surface may further comprise a polymer coating as disclosed elsewhere herein, and wherein said surface may further comprise one or more oligonucleotides such as a capture oligonucleotide, an adapter oligonucleotide, or any other oligonucleotide as disclosed herein. In some embodiments, a device of the present disclosure may comprise a first surface in an orientation generally facing the interior of the flow channel, a second surface in an orientation generally facing the interior of the flow channel and further generally facing or parallel to the first surface, a third surface generally facing the interior of a second flow channel, and a fourth surface, generally facing the interior of the second flow channel and generally opposed to or parallel to the third surface; wherein said second and third surfaces may be located on or attached to opposite sides of a generally planar substrate which may be a reflective, transparent, or translucent substrate. In some embodiments, an imaging surface or imaging surfaces within a flow cell may be located within the center of a flow cell or within or as part of a division between two subunits or subdivisions of a flow cell, wherein said flow cell may comprise a top surface and a bottom surface, one or both of which may be transparent to such detection mode as may be utilized; and wherein a surface comprising oligonucleotides or polynucleotides and/or one or more polymer coatings, may be placed or interposed within the lumen of the flow cell. In some embodiments, the top and/or bottom surfaces do not include attached oligonucleotides or polynucleotides. In some embodiments, said top and/or bottom surfaces do comprise attached oligonucleotides and/or polynucleotides. In some embodiments, either said top or said bottom surface may comprise attached oligonucleotides and/or polynucleotides. A surface or surfaces placed or interposed within the lumen of a flow cell may be located on or attached one side, an opposite side, or both sides of a generally planar substrate which may be a reflective, transparent, or translucent substrate. In some embodiments, an optical apparatus as provided elsewhere herein or as otherwise known in the art is utilized to provide images of a first surface, a second surface, a third surface, a fourth surface, a surface interposed within the lumen of a flow cell, or any other surface provided herein which may contain one or more oligonucleotides or polynucleotides attached thereto.

(c) Microfluidic Chip Flow Cell Cartridges

Also disclosed herein are microfluidic channel flow cell cartridges that may a plurality of independent flow channels. A non-limiting example of microfluidic chip flow cell cartridge that comprises a chip having two or more parallel glass channels formed on the chip, fluidic adaptors coupled to the chip, and a cartridge chassis that mates with the chip and/or fluidic adapters such that the chip is posited in a fixed orientation relative to the cartridge. In some instances, the fluidic adaptors may be integrated with the cartridge chassis. In some instances, the cartridge may comprise additional adapters that mate with the chip and/or fluidic adapters. In some instances, the chip is permanently mounted in the cartridge. In some instances, the cartridge chassis is designed to allow one or more chips of the flow cell cartridge to be interchangeable removed and replaced. For example, in some instances, the cartridge chassis may comprise a hinged “clamshell” configuration which allows it to be opened so that one or more capillaries may be removed and replaces. In some instances, the cartridge chassis is configured to mount on, for example, the stage of a microscope system or within a cartridge holder of an instrument system. Even through only one chip is described in the non-limiting example, it is understood that more than one chip can be used in the microfluidic channel flow cell cartridge

The flow cell cartridges of the present disclosure may comprise a single microfluidic chip or a plurality of microfluidic chips. In some instances, the flow cell cartridges of the present disclosure may comprise 2, 3, 4, 5, 6, 7, 8, 9, 10, 11, 12, 13, 14, 15, 16, 17, 18, 19, 20, or more than 20 microfluidic chips. In some instances, the microfluidic chip can have one channel. In some instances, the microfluidic chip can have 2, 3, 4, 5, 6, 7, 8, 9, 10, 11, 12, 13, 14, 15, 16, 17, 18, 19, 20, or more than 20 channels. The one or more chips of the flow cell cartridge may have any of the geometries, dimensions, material compositions, and/or coatings as described above for the single microfluidic chip flow cell devices. Similarly, the fluidic adapters for the individual chip in the cartridge (typically two fluidic adapters per capillary) may have any of the geometries, dimensions, and material compositions as described above for the single microfluidic chip flow cell devices, except that in some instances the fluidic adapters may be integrated directly with the cartridge chassis. In some instances, the cartridge may comprise additional adapters (i.e., in addition to the fluidic adapters) that mate with the chip and/or fluidic adapters and help to position the chip within the cartridge. These adapters may be constructed using the same fabrication techniques and materials as those outlined above for the fluidic adapters.

The cartridge chassis (or “housing”) may be fabricated from metal and/or polymer materials such as aluminum, anodized aluminum, polycarbonate (PC), acrylic (PMMA), or Ultem (PEI), while other materials are also consistent with the disclosure. A housing may be fabricated using CNC machining and/or molding techniques, and designed so that one, two, or more than two capillaries are constrained by the chassis in a fixed orientation to create independent flow channels. The capillaries may be mounted in the chassis using, e.g., a compression fit design, or by mating with compressible adapters made of silicone or a fluoroelastomer. In some instance, two or more components of the cartridge chassis (e.g., an upper half and a lower half) are assembled using, e.g., screws, clips, clamps, or other fasteners so that the two halves are separable. In some instances, two or more components of the cartridge chassis are assembled using, e.g., adhesives, solvent bonding, or laser welding so that the two or more components are permanently attached.

Some flow cell cartridges of the present disclosure further comprise additional components that are integrated with the cartridge to provide enhanced performance for specific applications. Examples of additional components that may be integrated into the cartridge include, but are not limited to, fluid flow control components (e.g., miniature valves, miniature pumps, mixing manifolds, etc.), temperature control components (e.g., resistive heating elements, metal plates that serve as heat sources or sinks, piezoelectric (Peltier) devices for heating or cooling, temperature sensors), or optical components (e.g., optical lenses, windows, filters, mirrors, prisms, fiber optics, and/or light-emitting diodes (LEDs) or other miniature light sources that may collectively be used to facilitate spectroscopic measurements and/or imaging of one or more capillary flow channels).

The flow cell devices and flow cell cartridges disclosed herein may be used as components of systems designed for a variety of chemical analysis, biochemical analysis, nucleic acid analysis, cell analysis, or tissue analysis application. In general, such systems may comprise one or more fluid flow control modules, temperature control modules, spectroscopic measurement and/or imaging modules, and processors or computers, as well as one or more of the single capillary flow cell devices and capillary flow cell cartridges or the microfluidic chip flow cell devices and flow cell cartridges described herein.

The systems disclosed herein may comprise 1, 2, 3, 4, 5, 6, 7, 8, 9, 10, or more than 10 single capillary flow cell devices or capillary flow cell cartridges. In some instances the single capillary flow cell devices or capillary flow cell cartridges may be removable, exchangeable components of the disclosed systems. In some instances, the single capillary flow cell devices or capillary flow cell cartridges may be disposable or consumable components of the disclosed systems. The systems disclosed herein may comprise 1, 2, 3, 4, 5, 6, 7, 8, 9, 10, or more than 10 single microfluidic channel flow cell devices or microfluidic channel flow cell cartridges. In some instances the single microfluidic channel flow cell devices or microfluidic channel flow cell cartridges may be removable, exchangeable components of the disclosed systems. In some instances, the flow cell devices or flow cell cartridges may be disposable or consumable components of the disclosed systems.

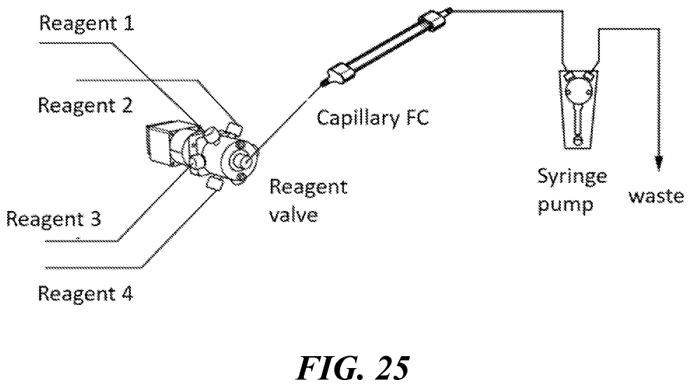

FIG. 25 illustrates one embodiment of a simple system comprising a single capillary flow cell connected to various fluid flow control components, where the single capillary is optically accessible and compatible with mounting on a microscope stage or in a custom imaging instrument for use in various imaging applications. A plurality of reagent reservoirs are fluidically-coupled with the inlet end of the single capillary flow cell device, where the reagent flowing through the capillary at any given point in time is controlled by means of a programmable rotary valve that allows the user to control the timing and duration of reagent flow. In this non-limiting example, fluid flow is controlled by means of a programmable syringe pump that provides precise control and timing of volumetric fluid flow and fluid flow velocity.

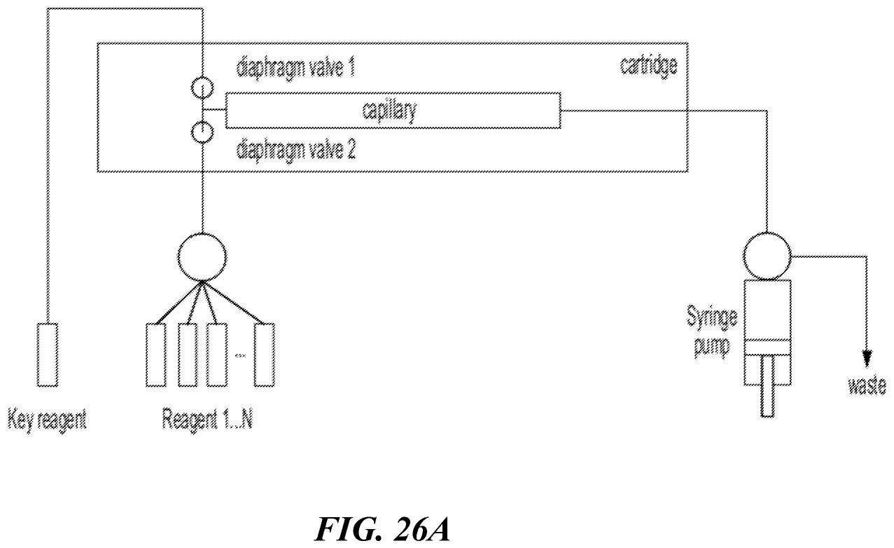

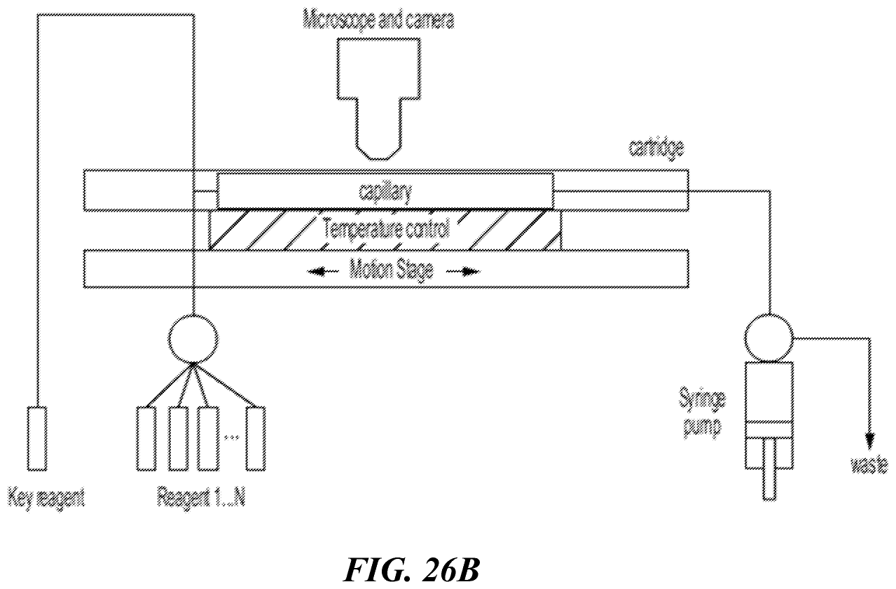

FIG. 26A illustrates one embodiment of a system that comprises a capillary flow cell cartridge having integrated diaphragm valves to minimize dead volume and conserve certain key reagents. The integration of miniature diaphragm valves into the cartridge allows the valve to be positioned in close proximity to the inlet of the capillary, thereby minimizing dead volume within the device and reducing the consumption of costly reagents. The integration of valves and other fluid control components within the capillary flow cell cartridge also allows greater fluid flow control functionality to be incorporated into the cartridge design.

FIG. 26B shows an example of a capillary flow cell cartridge-based fluidics system used in combination with a microscope setup, where the cartridge incorporates or mates with a temperature control component such as a metal plate that makes contact with the capillaries within the cartridge and serves as a heat source/sink. The microscope setup consists of an illumination system (e.g., including a laser, LED, or halogen lamp, etc., as a light source), an objective lens, an imaging system (e.g., a CMOS or CCD camera), and a translation stage to move the cartridge relative to the optical system, which allows, e.g., fluorescence and/or bright field images to be acquired for different regions of the capillary flow cells as the stage is moved.

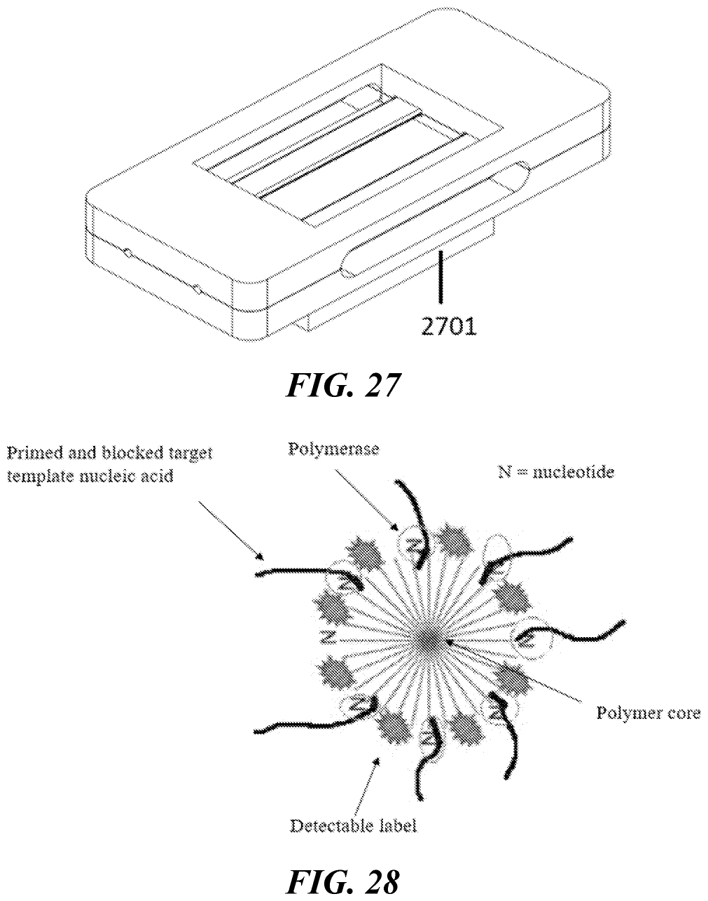

In some embodiments, the systems described herein provide for temperature control of the flow cells (e.g., capillary or microfluidic channel flow cells) through the use of a metal plate 2701 that is placed in contact with the flow cell cartridge of FIG. 24B, as shown in FIG. 27. In some instances, the metal plate may be integrated with the cartridge chassis. In some instances, the metal plate may be temperature controlled using a Peltier or resistive heater. In some embodiments, the system comprises a non-contact thermal control mechanism. In this approach, a stream of temperature-controlled air is directed through the flow cell cartridge (e.g., towards a single capillary flow cell device or a microfluidic channel flow cell device) using an air temperature control system. The air temperature control system comprises a heat exchanger, e.g., a resistive heater coil, fins attached to a Peltier device, etc., that is capable of heating and/or cooling the air and holding it at a constant, user-specified temperature. The air temperature control system also comprises an air delivery device, such as a fan, that directs the stream of heated or cooled air to the capillary flow cell cartridge. In some instances, the air temperature control system may be set to a constant temperature T1 so that the air stream, and consequently the flow cell or cartridge (e.g., capillary flow cell or microfluidic channel flow cell) is kept at a constant temperature Tz, which in some cases may differ from the set temperature T1 depending on the environment temperature, air flow rate, etc. In some instances, two or more such air temperature control systems may be installed around the capillary flow cell device or flow cell cartridge so that the capillary or cartridge may be rapidly cycled between several different temperatures by controlling which one of the air temperature control systems is active at a given time. In another approach, the temperature setting of the air temperature control system may be varied so the temperature of the capillary flow cell or cartridge may be changed accordingly.

Fluid Flow Control Module

In general, the disclosed instrument systems will provide fluid flow control capability for delivering samples or reagents to the one or more flow cell devices or flow cell cartridges (e.g., single capillary flow cell device or microfluidic channel flow cell device) connected to the system. Reagents and buffers may be stored in bottles, reagent and buffer cartridges, or other suitable containers that are connected to the flow cell inlets by means of tubing and valve manifolds. The disclosed systems may also include processed sample and waste reservoirs in the form of bottles, cartridges, or other suitable containers for collecting fluids downstream of the capillary flow cell devices or capillary flow cell cartridges. In some embodiments, the fluid flow control (or “fluidics”) module may provide programmable switching of flow between different sources, e.g. sample or reagent reservoirs or bottles located in the instrument, and the central region (e.g., capillary or microfluidic channel) inlet(s). In some embodiments, the fluid flow control module may provide programmable switching of flow between the central region (e.g., capillary or microfluidic channel) outlet(s) and different collection points, e.g., processed sample reservoirs, waste reservoirs, etc., connected to the system. In some instances, samples, reagents, and/or buffers may be stored within reservoirs that are integrated into the flow cell cartridge itself. In some instances, processed samples, spent reagents, and/or used buffers may be stored within reservoirs that are integrated into the flow cell cartridge itself.

Control of fluid flow through the disclosed systems will typically be performed through the use of pumps (or other fluid actuation mechanisms) and valves (e.g., programmable pumps and valves). Examples of suitable pumps include, but are not limited to, syringe pumps, programmable syringe pumps, peristaltic pumps, diaphragm pumps, and the like. Examples of suitable valves include, but are not limited to, check valves, electromechanical two-way or three-way valves, pneumatic two-way and three-way valves, and the like. In some embodiments, fluid flow through the system may be controlled by means of applying positive pneumatic pressure to one or more inlets of the reagent and buffer containers, or to inlets incorporated into flow cell cartridge(s) (e.g., capillary or microfluidic channel flow cell cartridges). In some embodiments, fluid flow through the system may be controlled by means of drawing a vacuum at one or more outlets of waste reservoir(s), or at one or more outlets incorporated into flow cell cartridge(s) (e.g., capillary or microfluidic channel flow cell cartridges).

In some instances, different modes of fluid flow control are utilized at different points in an assay or analysis procedure, e.g. forward flow (relative to the inlet and outlet for a given capillary flow cell device), reverse flow, oscillating or pulsatile flow, or combinations thereof. In some applications, oscillating or pulsatile flow may be applied, for example, during assay wash/rinse steps to facilitate complete and efficient exchange of fluids within the one or more flow cell devices or flow cell cartridges (e.g., single capillary flow cell devices or cartridges and microfluidic chip flow cell devices or cartridges).

Similarly, in some cases different fluid flow rates may be utilized at different points in the assay or analysis process workflow, for example, in some instances, the volumetric flow rate may vary from −100 ml/sec to +100 ml/sec. In some embodiment, the absolute value of the volumetric flow rate may be at least 0.001 ml/sec, at least 0.01 ml/sec, at least 0.1 ml/sec, at least 1 ml/sec, at least 10 ml/sec, or at least 100 ml/sec. In some embodiments, the absolute value of the volumetric flow rate may be at most 100 ml/sec, at most 10 ml/sec, at most 1 ml/sec, at most 0.1 ml/sec, at most 0.01 ml/sec, or at most 0.001 ml/sec. The volumetric flow rate at a given point in time may have any value within this range, e.g. a forward flow rate of 2.5 ml/sec, a reverse flow rate of −0.05 ml/sec, or a value of 0 ml/sec (i.e., stopped flow).

Temperature Control Module

As noted above, in some instances the disclosed systems will include temperature control functionality for the purpose of facilitating the accuracy and reproducibility of assay or analysis results. Examples of temperature control components that may be incorporated into the instrument system (or capillary flow cell cartridge) design include, but are not limited to, resistive heating elements, infrared light sources, Peltier heating or cooling devices, heat sinks, thermistors, thermocouples, and the like. In some instances, the temperature control module (or “temperature controller”) may provide for a programmable temperature change at a specified, adjustable time prior to performing specific assay or analysis steps. In some instances, the temperature controller may provide for programmable changes in temperature over specified time intervals. In some embodiments, the temperature controller may further provide for cycling of temperatures between two or more set temperatures with specified frequency and ramp rates so that thermal cycling for amplification reactions may be performed.

Spectroscopy or Imaging Modules

As indicated above, in some instances the disclosed systems will include optical imaging or other spectroscopic measurement capabilities. For example, any of a variety of imaging modes known to those of skill in the art may be implemented including, but not limited to, bright-field, dark-field, fluorescence, luminescence, or phosphorescence imaging. In some embodiments, the central region comprises a window that allows at least a part of the central region to be illuminated and imaged. In some embodiments, the capillary tube comprises a window that allows at least a part of the capillary tube to be illuminated and imaged. In some embodiments, the microfluidic chip comprises a window that allows at least a part of the chip channel to be illuminated and imaged.

In some embodiments, single wavelength excitation and emission fluorescence imaging may be performed. In some embodiments, dual wavelength excitation and emission (or multi-wavelength excitation or emission) fluorescence imaging may be performed. In some instances, the imaging module is configured to acquire video images. The choice of imaging mode may impact the design of the flow cells devices or flow cell cartridges in that all or a portion of the capillaries or cartridge will necessarily need to be optically transparent over the spectral range of interest. In some instances, a plurality of capillaries within a capillary flow cell cartridge may be imaged in their entirety within a single image. In some embodiments, only a single capillary or a subset of capillaries within a capillary flow cell cartridge, or portions thereof, may be imaged within a single image. In some embodiments, a series of images may be “tiled” to create a single high resolution image of one, two, several, or the entire plurality of capillaries within a cartridge. In some instances, a plurality of channels within a microfluidic chip may be imaged in their entirety within a single image. In some embodiments, only a single channel or a subset of channels within a microfluidic chip, or portions thereof, may be imaged within a single image. In some embodiments, a series of images may be “tiled” to create a single high resolution image of one, two, several, or the entire plurality of capillaries or microfluidic channels within a cartridge.

A spectroscopy or imaging module may comprise, e.g., a microscope equipped with a CMOS of CCD camera. In some instances, the spectroscopy or imaging module may comprise, e.g., a custom instrument configured to perform a specific spectroscopic or imaging technique of interest. In general, the hardware associated with the imaging module may include light sources, detectors, and other optical components, as well as processors or computers.

Light Sources

Any of a variety of light sources may be used to provide the imaging or excitation light, including but not limited to, tungsten lamps, tungsten-halogen lamps, arc lamps, lasers, light emitting diodes (LEDs), or laser diodes. In some instances, a combination of one or more light sources, and additional optical components, e.g. lenses, filters, apertures, diaphragms, mirrors, and the like, may be configured as an illumination system (or sub-system).

Detectors

Any of a variety of image sensors may be used for imaging purposes, including but not limited to, photodiode arrays, charge-coupled device (CCD) cameras, or complementary metal—oxide— semiconductor (CMOS) image sensors. As used herein, “imaging sensors” may be one-dimensional (linear) or two-dimensional array sensors. In many instances, a combination of one or more image sensors, and additional optical components, e.g. lenses, filters, apertures, diaphragms, mirrors, and the like, may be configured as an imaging system (or sub-system). In some instances, e.g., where spectroscopic measurements are performed by the system rather than imaging, suitable detectors may include, but are not limited to, photodiodes, avalanche photodiodes, and photomultipliers.

Other Optical Components

The hardware components of the spectroscopic measurement or imaging module may also include a variety of optical components for steering, shaping, filtering, or focusing light beams through the system. Examples of suitable optical components include, but are not limited to, lenses, mirrors, prisms, apertures, diffraction gratings, colored glass filters, long-pass filters, short-pass filters, bandpass filters, narrowband interference filters, broadband interference filters, dichroic reflectors, optical fibers, optical waveguides, and the like. In some instances, the spectroscopic measurement or imaging module may further comprise one or more translation stages or other motion control mechanisms for the purpose of moving capillary flow cell devices and cartridges relative to the illumination and/or detection/imaging sub-systems, or vice versa.

Total Internal Reflection

In some instances, the optical module or sub-system may be designed to use all or a portion of an optically transparent wall of the capillaries or microfluidic channels in flow cell devices and cartridges as a waveguide for delivering excitation light to the capillary or channel lumen(s) via total internal reflection. When incident excitation light strikes the surface of the capillary or channel lumen at an angle with respect to a normal to the surface that is larger than the critical angle (determined by the relative refractive indices of the capillary or channel wall material and the aqueous buffer within the capillary or channel), total internal reflection occurs at the surface and the light propagates through the capillary or channel wall along the length of the capillary or channel Total internal reflection generates an evanescent wave at the lumen surface which penetrates the lumen interior for extremely short distances, and which may be used to selectively excite fluorophores at the surface, e.g., labeled nucleotides that have been incorporated by a polymerase into a growing oligonucleotide through a solid-phase primer extension reaction.

Imaging Processing Software

In some instances, the system may further comprise a computer (or processor) and computer-readable medium that includes code for providing image processing and analysis capability. Examples of image processing and analysis capability that may be provided by the software include, but are not limited to, manual, semi-automated, or fully-automated image exposure adjustment (e.g. white balance, contrast adjustment, signal-averaging and other noise reduction capability, etc.), automated edge detection and object identification (e.g., for identifying clonally-amplified clusters of fluorescently-labeled oligonucleotides on the lumen surface of capillary flow cell devices), automated statistical analysis (e.g., for determining the number of clonally-amplified clusters of oligonucleotides identified per unit area of the capillary lumen surface, or for automated nucleotide base-calling in nucleic acid sequencing applications), and manual measurement capabilities (e.g. for measuring distances between clusters or other objects, etc.). Optionally, instrument control and image processing/analysis software may be written as separate software modules. In some embodiments, instrument control and image processing/analysis software may be incorporated into an integrated package.

System control software: In some instances, the system may comprise a computer (or processor) and a computer-readable medium that includes code for providing a user interface as well as manual, semi-automated, or fully-automated control of all system functions, e.g., control of the fluidics module, the temperature control module, and/or the spectroscopy or imaging module, as well as other data analysis and display options. The system computer or processor may be an integrated component of the system (e.g. a microprocessor or mother board embedded within the instrument) or may be a stand-alone module, for example, a main frame computer, a personal computer, or a laptop computer. Examples of fluid control functions provided by the system control software include, but are not limited to, volumetric fluid flow rates, fluid flow velocities, the timing and duration for sample and reagent addition, buffer addition, and rinse steps. Examples of temperature control functions provided by the system control software include, but are not limited to, specifying temperature set point(s) and control of the timing, duration, and ramp rates for temperature changes. Examples of spectroscopic measurement or imaging control functions provided by the system control software include, but are not limited to, autofocus capability, control of illumination or excitation light exposure times and intensities, control of image acquisition rate, exposure time, and data storage options.

Processors and Computer Systems

In some instances, the disclosed methods and systems may utilize or comprise one or more processors or computers. The processor may be a hardware processor such as a central processing unit (CPU), a graphic processing unit (GPU), a general-purpose processing unit, or a computing platform. The processor may be comprised of any of a variety of suitable integrated circuits, microprocessors, logic devices, field-programmable gate arrays (FPGAs) and the like. In some instances, the processor may be a single core or multi core processor, or a plurality of processors may be configured for parallel processing. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations.

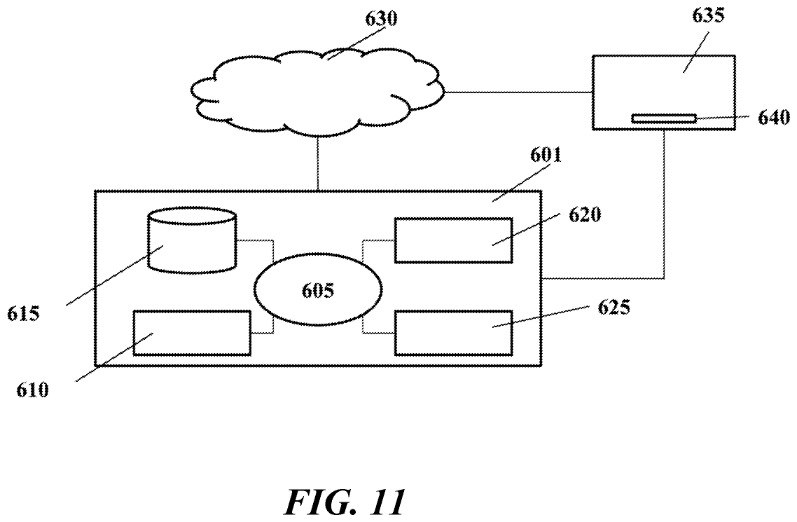

In some embodiment, such processors and computer systems are programmed to implement methods of the disclosure. FIG. 11 shows a computer system 601 that is programmed or otherwise configured to implement methods of the disclosure. The computer system 601 can regulate various aspects of the present disclosure, such as, for example, controlling the experiment conditions of generating the circular nucleic acid molecule, analyzing the target nucleic acid molecule, and optimizing the experiment conditions of generating the circular nucleic acid library. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, parameters of on-going experiments, and information regarding the nucleic acid sequencing. Examples of UI's include, without limitation, a graphical user interface (GUI) and web-based user interface.

Methods of the present disclosure can be implemented by way of one or more algorithms. An algorithm can be implemented by way of software upon execution by the central processing unit 605. The algorithm can, for example, analyze big sequence data and simulate biochemical reaction networks.

### Library Preparation Kits

Disclosed herein are kits for use to prepare a nucleic acid sequencing library and/or sequence the nucleic acid sequencing library. In some embodiments, the kits comprise compositions described herein, such as reagents and substrates for circularizing a nucleic acid molecule and/or sequencing the nucleic acid molecule following circularization.

The kit may include enzymes, nucleic acids, nucleotides, supports with functionalized surfaces, or instructions. In some embodiments, the enzymes may be ligating enzymes, proteases, transposases, any one of enzymes described herein and combination thereof. In some embodiments, the nucleic acids may be oligonucleotides, splint oligonucleotides, any oligonucleotides or nucleic acids described herein, or any combinations thereof. In some embodiments, nucleotides may comprise nucleotides with blocking moieties. In some embodiments, nucleotides may comprise polymer-nucleotide conjugates. In some embodiments, nucleotides may comprise detection moieties. In some embodiments, supports with functionalized surfaces may comprise a plastic, metal, glass, or any combinations thereof for the support. In some embodiments, supports with functionalized surfaces may comprise hydrophilic, hydrophobic, polymeric, primed, or any combinations thereof for the functionalizations.

In some embodiments, the instructions may comprise a description for a method of circularizing single stranded nucleic acid, single stranded DNA, single stranded RNA, double stranded nucleic acid, double stranded DNA, double stranded RNA, any nucleic acid described herein and combinations thereof. In some embodiments, the instructions may further comprise a description for a method of attaching nucleic acid adapters or primers before circularization, simultaneously with circularization, or after circularization. In some embodiments, the instructions may further comprise a description for processing the genetic material from a biological source. In some embodiments, the instructions may comprise a description for detecting nucleic acid sequences. In some embodiments, the instructions may comprise a description for planning multiple stages, each stage employing one of the methods described herein. For example, one embodiment of such a description may describe the steps of fragmenting single-stranded RNA to a plurality of shorter target single stranded RNA, attaching an amplification adaptor sequence to both the 5′ end and the 3′ end to each of the plurality of target single stranded RNA, attaching splint nucleic acid recognition sequence to both the 5′ end and the 3′ end to each of the plurality of target single stranded RNA, carrying out splint ligation with enzymes to circularize the plurality of target RNA, amplifying the plurality of target circularized RNA, immobilizing the plurality of target circularized RNA onto a hydrophilic surface, and then determining the sequence of the target circularized RNA on the surface with labeled nucleotides. In another example, one embodiment of such a description may describe the steps of fragmenting double stranded DNA to a plurality of target single stranded DNA, attaching an amplification adaptor sequence to both the 5′ end and the 3′ end to each of the plurality of target single stranded DNA, attaching an immobilization adaptor sequence to both the 5′ end and the 3′ end to each of the plurality of target single stranded DNA, immobilizing the plurality of target single-stranded DNA to a surface, attaching splint nucleic acid recognition sequence to both the 5′ end and the 3′ end to each of the plurality of target single stranded DNA, carrying out splint ligation with enzymes to circularize the plurality of target DNA, rolling-circle amplifying the plurality of target circularized DNA, and then determining the sequence of the target circularized DNA on the surface with labeled nucleotides. The embodiments described herein are not limiting examples.

### Numbered Embodiments

Embodiment 1. An embodiment disclosed herein comprises methods for processing a nucleic acid comprising: (a) providing a double-stranded nucleic acid or fragment thereof; (b) coupling an adapter molecule to a 5′ end of at least one strand of the double-stranded nucleic acid molecule or fragment thereof with a transposase; and (c) adding one or more nucleic acids to the at least one strand of the double-stranded nucleic acid molecule or fragment thereof thereby forming a circular nucleic acid molecule. Embodiment 2. The method of embodiment 1, wherein the double-stranded nucleic acid or fragment thereof is deoxyribonucleic acid. Embodiment 3. The method of embodiment 1 or 2, wherein the adapter molecule is a hairpin adapter molecule. Embodiment 4. The method of any one of embodiments 1-3, wherein the adapter molecule comprises at least one unnatural nucleic acid configured to participate in a G-quadruplex. Embodiment 5. The method of any one of embodiments 1-4, wherein the adapter molecule is coupled to a surface-bound nucleic acid molecule coupled to a surface. Embodiment 6. The method of embodiment 5, wherein the adapter molecule is coupled to the surface-bound nucleic acid molecule by nucleic acid hybridization. Embodiment 7. The method of embodiment 5 or 6, wherein the surface-bound nucleic acid molecule comprises at least one unnatural nucleic acid configured to participate in the G-quadruplex. Embodiment 8. The method of any one of embodiments 5-7, wherein the surface-bound nucleic acid molecule comprises a transposon associated with the transposase. Embodiment 9. The method of any one of embodiments 1-8, wherein the adapter molecule comprises a transposon associated with the transposase. Embodiment 10. The method of any one of embodiments 1-8, wherein the transposase is Transposase 5. Embodiment 11. The method of any one of embodiments 1-10, wherein one or more of the transposase and the adapter molecule is coupled to a surface. Embodiment 12. The method of embodiment 11, wherein forming the circular nucleic acid molecule occurs in a discrete region of the surface. Embodiment 13. The method of any one of embodiment 1-12, wherein the circular nucleic acid molecule is a single-stranded circular nucleic acid molecule. Embodiment 14. The method of any one of embodiments 1-12, wherein the circular nucleic acid molecule is a double-stranded circular nucleic acid molecule. Embodiment 15. The method of any one of embodiments 1-14, wherein the at least one strand of the double-stranded nucleic acid or fragment thereof is a sense strand. Embodiment 16. The method of any one of embodiments 1-15, further comprising forming the circular nucleic acid molecule. Embodiment 17. The method of any one of embodiments 1-16, further comprising coupling the adapter molecule to the 5′ end of both strands of the double-stranded nucleic acid or fragment thereof. Embodiment 18. The method of any one of embodiments 1-17, further comprising forming two circular nucleic acid molecules comprising a first circular nucleic acid molecule and a second circular nucleic acid molecule, wherein said first circular nucleic acid molecule comprises a sense strand of the double-stranded nucleic acid molecule or fragment thereof, and wherein said second circular nucleic acid molecule comprises a corresponding antisense strand of the double-stranded nucleic acid molecule or fragment thereof. Embodiment 19. The method of embodiment 18, wherein the first circular nucleic acid molecule is formed on a first discrete region of a surface and the second circular nucleic acid molecule is formed on a second discrete region of the surface. Embodiment 20. The method of any one of embodiments 1-19, wherein the at least one strand of the double-stranded nucleic acid molecule or fragment thereof is sequenced. Embodiment 21. The method of embodiment 20, wherein the at least one strand of the double-stranded nucleic acid molecule or fragment thereof is sequenced in 10 minutes or less. Embodiment 22. The method of any one of embodiments 20-21, further comprising synthesizing a complementary strand comprising a nucleic acid sequence that is the reverse complement to a nucleic acid sequence of the at least one strand of the double-stranded nucleic acid molecule or fragment thereof. Embodiment 23. The method of embodiment 22, further comprising: (a) removing the at least one strand of the double stranded nucleic acid molecule or fragment thereof; and (b) sequencing the complementary strand. Embodiment 24. The method of embodiment 23, wherein removing in (a) is performed enzymatically. Embodiment 25. The method of embodiment 22, further comprising: (a) displacing the complementary strand from the at least one strand of the double-stranded nucleic acid molecule or fragment thereof spatially such that the complementary strand and the at least one strand of the double-stranded nucleic acid molecule or fragment thereof do not anneal; and (b) sequencing the complementary strand and the at least one strand of the double stranded nucleic acid molecule or fragment thereof. Embodiment 26. The method of embodiment 25, wherein displacing in (a) is performed enzymatically. Embodiment 27. The method of embodiment 25, wherein sequencing of the complementary strand and sequencing of the at least one strand of the double stranded nucleic acid molecule or fragment thereof occurs substantially simultaneously. Embodiment 28. The method of embodiment 27, performed in half of an amount of time of a comparable sequencing reaction that does not sequence the complementary strand and the at least one strand of the double stranded nucleic acid molecule or fragment thereof simultaneously. Embodiment 29. The method of embodiment 25, wherein sequencing of the complementary strand and sequencing of the at least one strand of the double stranded nucleic acid molecule or fragment thereof occurs substantially sequentially in 20 minutes or less. Embodiment 30. The method of any one or embodiments 1-29, further comprising amplifying the circular nucleic acid molecule using rolling circle amplification.

Embodiment 31. An embodiment disclosed herein comprises methods for generating a circular nucleic acid molecule, comprising: (a) providing two double-stranded enzyme recognition nucleic acid molecule, a double-stranded target nucleic acid molecule, and one or more adaptors, wherein at least one adaptor comprises a universal primer site, a surface binding site, or an index site; (b) joining one of the two double-stranded enzyme recognition nucleic acid molecules to one end of the double-stranded target nucleic acid molecule, and another one of the two double-stranded enzyme recognition nucleic acid molecules to another end of the double-stranded target nucleic acid molecule, to form a joint double-stranded nucleic acid molecule, wherein the joint double-stranded nucleic acid molecule comprises the at least one adaptor between the one of the two double-stranded enzyme recognition nucleic acid molecule and the double-stranded target nucleic acid molecule; and (c) contacting the joint double-stranded nucleic acid molecule to an enzyme, wherein the enzyme binds to the two double-stranded enzyme recognition nucleic acid molecules to form the circular nucleic acid molecule. Embodiment 32. The method for generating the circular nucleic acid molecule of embodiment 31, wherein the enzyme cleaves the double-stranded enzyme recognition nucleic acid molecule. Embodiment 33. The method for generating the circular nucleic acid molecule of embodiment 32, wherein, after the cleavage, cleavage ends of the double-stranded enzyme recognition nucleic acid molecule form hairpin structures. Embodiment 34. The method for generating the circular nucleic acid molecule of any one of embodiments 31-33, wherein the enzyme is a protelomerase. Embodiment 35. The method for generating the circular nucleic acid molecule of embodiment 34, wherein the protelomerase is Te1N protelomerase. Embodiment 36. The method for generating the circular nucleic acid molecule of any one of embodiments 31-35, wherein the joining is carried out by a nucleic acid polymerase. Embodiment 37. The method for generating the circular nucleic acid molecule of embodiment 35, wherein the Te1N protelomerase comprises an amino acid sequence of SEQ ID NO: 1. Embodiment 38. The method for generating the circular nucleic acid molecule of any one of embodiments 31-37, wherein the surface binding site is configured to immobilize the circular nucleic acid molecule to a surface. Embodiment 39. The method for generating the circular nucleic acid molecule of embodiment 38, wherein the surface is a surface of a support or a surface within a support. Embodiment 40. The method for generating the circular nucleic acid molecule of any one of embodiments 31-39, wherein the at least one adaptor is inserted between the double-stranded enzyme recognition nucleic acid molecule and the double-stranded target nucleic acid molecule by a transposase. Embodiment 41. The method for generating the circular nucleic acid molecule of any one of embodiments 31-39, wherein the at least one adaptor is ligated to the one double-stranded target nucleic acid molecule by a ligase before the joining. Embodiment 42. The method for generating the circular nucleic acid molecule of any one of embodiments 31-41, wherein the at least one adaptor further comprises a P5 site or a P7 site.

Embodiment 43. A method for generating a circular nucleic acid library, comprising: (a) fragmenting a double-stranded nucleic acid sample to form a plurality of double-stranded nucleic acid fragments; (b) joining a plurality of enzyme recognition nucleic acid molecules to the plurality of double-stranded nucleic acid fragments to form a plurality of joint double-stranded nucleic acid molecules, such that at least one joint double-stranded nucleic acid molecules has at least one enzyme recognition nucleic acid molecule on each end of a given double-stranded nucleic acid fragment; and (c) contacting a given joint double-stranded nucleic acid molecule with at least one enzyme recognition nucleic acid molecule on each end to an enzyme, wherein the enzyme cleaves the at least one enzyme recognition nucleic acid molecule and rejoins cleavage ends of the at least one enzyme recognition nucleic acid molecule; (d) repeating (c), thereby generating the circular nucleic acid library from the double stranded nucleic acid sample. Embodiment 44. The method of embodiment 43, wherein the circular nucleic acid library comprises at least 100 circular nucleic acid molecules with distinguishable sequences. Embodiment 45. The method of embodiment 44, wherein the circular nucleic acid library comprises at least 1,000 circular nucleic acid molecules with distinguishable sequences. Embodiment 46. The method of embodiment 45, wherein the circular nucleic acid library comprises at least 10,000 circular nucleic acid molecules with distinguishable sequences. Embodiment 47. The method of embodiment 46, wherein the circular nucleic acid library comprises at least 100,000 circular nucleic acid molecules with distinguishable sequences. Embodiment 48. The method of any one of embodiments 43-47, wherein the fragmenting comprises shearing, sonicating, restriction digesting, and chemical digesting. Embodiment 49. The method of embodiment 48, wherein the shearing comprises acoustic shearing, point-sink shearing, and needle shearing. Embodiment 50. The method of any one of embodiments 43-49, wherein the fragmenting further comprises end repair. Embodiment 51. The method of any one of embodiments 43-50, wherein the fragmenting further comprises sticky end generation. Embodiment 52. The method of any one of embodiments 43-51, wherein the fragmenting further comprises overhang generation. Embodiment 53. The method of embodiment 52, wherein the overhang generation comprises 5′ end generation. Embodiment 54. The method of embodiment 53, wherein the overhang generation comprises 3′ end generation. Embodiment 55. The method of any one of embodiments 43-54, wherein the enzyme comprises a first enzyme that cleaves the at least one enzyme recognition nucleic acid molecule and a second enzyme that rejoins cleavage ends of the at least one enzyme recognition nucleic acid molecule. Embodiment 56. The method of any one of embodiments 43-55, wherein the rejoining comprises forming hairpin structures. Embodiment 57. The method of any one of embodiments 43-56, wherein the enzyme is a protelomerase. Embodiment 58. The method of embodiment 57, wherein the protelomerase is Te1N protelomerase. Embodiment 59. The method of any one of embodiments 43-58, wherein the joining is carried out by a nucleic acid polymerase. Embodiment 60. The method of embodiment 58, wherein the Te1N protelomerase comprises an amino acid sequence of SEQ ID NO: 1. Embodiment 61. The method of any one of embodiments 43-60, wherein the given joint double-stranded nucleic acid molecule comprises at least one adaptor between the at least one enzyme recognition nucleic acid molecule and the given double-stranded nucleic acid segment. Embodiment 62. The method of embodiment 61, wherein the at least one adaptor comprises a universal primer site, a surface binding site, or an index site. Embodiment 63. The method of embodiment 62, wherein the at least one adaptor further comprises a P5 site or a P7 site. Embodiment 64. The method of embodiment 62 or 63, wherein the surface binding site is configured to immobilize the circular nucleic acid molecule to a surface. Embodiment 65. The method of embodiment 64, wherein the surface is a surface of a support or a surface within a support. Embodiment 66. The method of embodiments 43-65, wherein the at least one adaptor is inserted between the at least one enzyme recognition nucleic acid molecule and the given double-stranded nucleic acid fragment by a transposase. Embodiment 67. The method of embodiments 43-65, wherein the at least one adaptor is ligated to the given double-stranded nucleic acid fragment by a ligase before the joining. Embodiment 68. The method of any one of embodiments 43-67, further comprising modifying the plurality of double-stranded nucleic acid fragments. Embodiment 69. The method of embodiment 68, wherein the modifying comprises repairing and A tailing. Embodiment 70. The method of any one of embodiments 43-69, further comprising sequencing the plurality of circular nucleic acid molecules. Embodiment 71. The method of embodiments 43-70, further comprising separating the plurality of circular nucleic acid molecules. Embodiment 72. The method of any one of embodiments 43-71, wherein the method takes at most 5 hours to complete. Embodiment 73. The method of embodiment 72, wherein the method takes at most 3 hours to complete. Embodiment 74. The method of embodiment 73, wherein the method takes at most 1 hour to complete. Embodiment 75. The method of embodiment 74, wherein the method takes at most 30 minutes to complete. Embodiment 76. The method of any one of embodiments 43-75, wherein the method is performed under isothermal amplification conditions. Embodiment 77. The method of any one of embodiments 43-76, further comprising clonal amplification of the plurality of circular nucleic acid molecules. Embodiment 78. The method of embodiment 77, wherein the clonal amplification comprises rolling circle amplification.

Embodiment 79. An embodiment disclosed herein comprising Y adaptors comprising at least part of an enzyme recognition nucleic acid molecule, a universal primer site, a surface binding site, and an index site. Embodiment 80. The Y adaptor of embodiment 79, further comprising a P5 site or a P7 site.

Embodiment 81. An embodiment disclosed herein comprising a hairpin adaptor comprising at least part of an enzyme recognition nucleic acid molecule, a universal primer site, a surface binding site, and an index site. Embodiment 82. The hairpin adaptor of embodiment 81, further comprising a P5 site or a P7 site.

Embodiment 83. An embodiment disclosed herein comprising methods for generating a nucleic acid library, comprising; (a) providing a double stranded nucleic acid comprising a target sequence; (b) ligating the ends of said sequence to produce a circular single stranded nucleic acid; and (c) replicating said sequence to produce one or more copies of said target sequence. Embodiment 84. The method of embodiment 83, wherein said ligating comprises attaching a single stranded or partially single stranded adapter to said double stranded nucleic acid comprising a target sequence. Embodiment 85. The method of embodiment 84, wherein said adapter comprises a hairpin. Embodiment 86. The method of any of embodiments 84-85, wherein said adapter comprises an annealing site for a sequencing primer. Embodiment 87. The method of any of embodiments 84-86, wherein said adapter comprises an annealing site for a capture oligonucleotide. Embodiment 88. The method of any of embodiments 83-87, wherein said replicating comprises rolling circle amplification. Embodiment 89. The method of any of embodiments 83-88, wherein said replicating occurs while said circular single stranded nucleic acid is attached to, bound to, or associated with, a low binding surface. Embodiment 90. The method of any of embodiments 83-90, further comprising a buffer the comprises one or more of acetonitrile or formamide. Embodiment 91. The method of any of embodiments 83-90, further comprising a buffer that comprises PEG. Embodiment 92. The method of embodiment 89, wherein said low binding surface comprises PEG. Embodiment 93. The method of embodiment 89, wherein said low binding surface comprises a capture oligonucleotide having one or more sequences complementary to one or more sequences of the circular single stranded nucleic acid. Embodiment 94. The method of any of embodiments 89 or 92-93, wherein said low binding surface comprises a capture oligonucleotide having one or more sequences complementary to the one or more copies of said target sequence. Embodiment 95. The method of any of embodiments 89 or 92-94, wherein said low binding surface comprises a capture oligonucleotide having one or more sequences complementary to one or more sequences of the nucleic acid library.

Embodiment 96. A method of nucleic acid processing, said method comprising: (a) providing a primed circular nucleic acid sequence coupled a surface comprising a hydrophilic polymer layer; (b) bringing said primed circular nucleic acid sequence or a derivative thereof into contact with one or more nucleotide moieties under conditions sufficient to form a stable binding complex between said one or more nucleotide moieties and a nucleotide of said primed circular nucleic acid sequence or said derivative thereof without incorporating said one or more nucleotide moieties into said primed circular nucleic acid sequence; and (c) detecting said stable multivalent binding complex to determine said identity of said nucleotide. Embodiment 97. The method of embodiment 96, further comprising bringing a fluid composition comprising said primed circular nucleic acid sequence in a concentration of less than or equal to about 1 nanomolar (nM) into contact with said surface under conditions sufficient to couple said primed circular nucleic acid sequence to said surface. Embodiment 98. The method of embodiment 97, wherein said concentration comprises less than or equal to about 100 picomolar (pM). Embodiment 99. The method of embodiment 97, wherein said concentration comprises less than or equal to about 80 picomolar (pM). Embodiment 100. The method of embodiment 97, wherein said concentration comprises between about 20 pM and about 1 nM. Embodiment 101. The method of embodiment 96, wherein said primed circular nucleic acid sequence is coupled to said surface at a surface density of more than or equal to about 10,000 primed circular nucleic acid sequences per micrometer (μm)². Embodiment 102. The method of embodiment 101, wherein said surface density comprises less than or equal to about 600,000 primed circular nucleic acid sequences per μm². Embodiment 103. The method of embodiment 96, wherein a plurality of colonies comprising said primed circular nucleic acid sequence or said derivative thereof is present at said surface with a colony density of more than or equal to about 300 K/mm². Embodiment 104. The method of embodiment 103, wherein said colony density comprises less than or equal to about 500 K/mm². Embodiment 105. The method of embodiment 96, wherein said primed circular nucleic acid sequence or said derivative thereof comprises one or more adaptors comprising an index site having a sequence complementary to at least a portion of a capture nucleic acid molecule coupled to said at least one polymer layer. Embodiment 106. The method of embodiment 105, wherein said index site comprises fewer than or equal to about 25 contiguous nucleotides. Embodiment 107. The method of embodiment 105, wherein said index site comprises fewer than or equal to about 10 contiguous nucleotides. Embodiment 108. The method of embodiment 105, wherein said index site comprises between about 5 and about 25 contiguous nucleotides. Embodiment 109. The method of embodiment 96, wherein said hydrophilic polymer layer comprises a polymer selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. Embodiment 110. The method of embodiment 96, wherein said one or more nucleotide moieties is coupled to a polymer core in one or more polymer-nucleotide compositions. Embodiment 111. The method of embodiment 110, wherein said one or more polymer-nucleotide conjugate compositions comprises a polymer core and a detectable label coupled thereto. Embodiment 112. The method of embodiment 96, wherein said primed circular nucleic acid sequence or said derivative thereof comprises a concatemer of two or more repeats of an identical sequence. Embodiment 113. The method of embodiment 96, further comprising: (d) amplifying said primed circular nucleic acid sequence using rolling circle amplification (RCA) prior to (c) to produce said derivative thereof. Embodiment 114. The method of embodiment 96, further comprising: (d) performing a primer extension reaction on said primed circular nucleic acid sequence or said derivative thereof; and (e) repeating (a) to (d) for each successive nucleotide to identify more than or equal to about 90% of said primed circular nucleic acid sequence or said derivative thereof. Embodiment 115. The method of embodiment 114, further comprising: (f) performing (a) to (e) in less than or equal to about 30 minutes. Embodiment 116. The method of embodiment 110, wherein said one or more polymer-nucleotide conjugate compositions comprises two or more types of said one or more polymer-nucleotide conjugate compositions. Embodiment 117. The method of embodiment 110, wherein said one or more polymer-nucleotide conjugate compositions comprises three or more types of said one or more polymer-nucleotide conjugate compositions. Embodiment 118. The method of embodiment 110, wherein said one or more polymer-nucleotide conjugate compositions comprises four types of said one or more polymer-nucleotide conjugate compositions. Embodiment 119. The method of any one or embodiment 116-118, wherein each of said types of said one or more polymer-nucleotide conjugate compositions comprises a nucleotide moiety with a distinct nucleobase type. Embodiment 120. The method of any one or embodiments 116-118, wherein each of said types of said one or more polymer-nucleotide conjugate compositions comprises a distinct detectable label.

Embodiment 121. A system comprising: a conjugated nucleotide composition comprising a polymer core and a plurality of nucleotide moieties attached thereto; and a solid support comprising a surface having a plurality of primed circular nucleic acid sequences coupled thereto, wherein at least a subset of said nucleotide moieties of said conjugated nucleotide composition is coupled to a subset of said plurality of said primed circular nucleic acid sequences. Embodiment 122. The system of embodiment 121, wherein said surface comprises a hydrophilic polymer coating. Embodiment 123. The system of embodiment 122, wherein said hydrophilic polymer coating comprises a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. Embodiment 124. The system of embodiments 121-122, wherein said hydrophilic polymer coating comprises a polymer having a molecular weight of more than or equal to about 1,000 Daltons. Embodiment 125. The system of embodiment 121, wherein said polymer core comprises a polymer elected from the coup consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. Embodiment 126. The system of embodiment 121-125, wherein said polymer core has a molecular weight of up to 10,000 Daltons, up to 20,000 Daltons, up to 30,000 Daltons, up to 40,000 Daltons, up to 50,000 Daltons, up to 60,000 Daltons, up to 70,000 Daltons, or greater than 70,000 Daltons. Embodiment 127. The system of embodiment 121-125, wherein said polymer core has a molecular weight of greater than 70,000 Daltons, greater than 60,000 Daltons, greater than 50,000 Daltons, greater than 40,000 Daltons, greater than 30,000 Daltons, greater than 20,000 Daltons, greater than 10,000 Daltons, or less than 10,000 Daltons. Daltons. Embodiment 128. The system of embodiment 121-127, wherein said plurality of said nucleotide moieties do not comprise a blocking group at a 3′ position of a sugar of said plurality of said nucleotide moieties. Embodiment 129. The system of embodiment 121-128, wherein said plurality of said primed circular nucleic acid sequences comprise a concatemer of two or more repeats of the same sequence. Embodiment 130. The system of embodiment 121-129, further comprising two or more of said polymer-nucleotide conjugate composition comprising a first polymer-nucleotide conjugate composition and a second polymer-nucleotide conjugate composition, wherein said first polymer-nucleotide conjugate composition comprises a nucleotide moiety having nucleobase type that differs from a nucleobase type of a nucleotide moiety of said second polymer-nucleotide conjugate composition. Embodiment 131. The system of embodiment 121-130, further comprising three or more of said polymer-nucleotide conjugate composition comprising a first polymer-nucleotide conjugate composition, a second polymer-nucleotide conjugate composition, and a third polymer-nucleotide conjugate composition, wherein each of said first polymer-nucleotide conjugate composition, said second polymer-nucleotide conjugate composition, and said third polymer-nucleotide conjugate composition comprises a nucleotide moiety having a distinct nucleobase type. Embodiment 132. The system of embodiment 121-131, further comprising four of said polymer-nucleotide conjugate composition comprising a first polymer-nucleotide conjugate composition, a second polymer-nucleotide conjugate composition, a third polymer-nucleotide conjugate composition, and a fourth polymer-nucleotide conjugate composition, wherein each of said first polymer-nucleotide conjugate composition, said second polymer-nucleotide conjugate composition, said third polymer-nucleotide conjugate composition, and said fourth polymer-nucleotide conjugate composition comprises a nucleotide moiety having a distinct nucleobase type. Embodiment 133. The system of embodiment 121-132, wherein a primed circular nucleic acid sequences of said plurality of said primed circular nucleic acid sequences comprises one or more unique molecular identifiers (UMI). Embodiment 134. The system of embodiment 121-133, further comprising a single-stranded oligonucleotide molecule, wherein a 5′ end and a 3′ end of said single-stranded oligonucleotide molecule is coupled to a 3′ end and a 5′ end of said primed circular nucleic acid sequences, respectively.

Embodiment 135. The system of embodiment 134, further comprising derivatives of said primed circular nucleic acid sequence. Embodiment 136. The system of embodiment 135, wherein said single-stranded oligonucleotide molecule is incorporated into the derivative. Embodiment 137. The system of embodiment 135, wherein said single-stranded oligonucleotide molecule is not incorporated into the derivative. Embodiment 138. The system of embodiment 134, wherein said single-stranded oligonucleotide molecule comprises between about 20-30 contiguous nucleotides. Embodiment 139. The system of embodiment 121-138, wherein a primed circular nucleic acid sequences of said plurality of said primed circular nucleic acid sequences comprises one or more adaptors comprising an index site having a nucleic acid sequence corresponding to at least a portion of a capture nucleic acid molecule coupled to said surface. Embodiment 140. The system of embodiment 139, wherein said index site comprises less than or equal to about 25 contiguous nucleotides. Embodiment 141. The system of embodiment 139, wherein said index site comprises less than or equal to about 10 contiguous nucleotides. Embodiment 142. The system of embodiment 139, wherein said index site comprises between about 5 and 25 contiguous nucleotides.

Embodiment 143. A composition comprising: a polymer core; and a plurality of nucleotide moieties coupled to said polymer core, wherein at least a subset of said nucleotide moieties is coupled to one or more primed circular nucleic acid sequences coupled to a surface. Embodiment 144. The composition of embodiment 143, wherein said surface comprises a hydrophilic polymer coating. Embodiment 145. The composition of embodiment 144, wherein said hydrophilic polymer coating comprises a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. Embodiment 146. The composition of embodiment 143-144, wherein said hydrophilic polymer coating comprises a polymer having a molecular weight of more than or equal to about 1,000 Daltons. Embodiment 147. The composition of embodiment 143-146, wherein said polymer core comprises a polymer elected from the coup consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran. Embodiment 148. The composition of embodiment 143-147, wherein said polymer core comprises a polymer has a molecular weight of up to 10,000 Daltons, up to 20,000 Daltons, up to 30,000 Daltons, up to 40,000 Daltons, up to 50,000 Daltons, up to 60,000 Daltons, up to 70,000 Daltons, or greater than 70,000 Daltons, Daltons. Embodiment 149. The composition of embodiment 143-148, wherein said polymer core comprises a polymer has a molecular weight of greater than 70,000 Daltons, greater than 60,000 Daltons, greater than 50,000 Daltons, greater than 40,000 Daltons, greater than 30,000 Daltons, greater than 20,000 Daltons, greater than 10,000 Daltons, or less than 10,000 Daltons. Daltons. Embodiment 150. The composition of embodiment 143-149, wherein said plurality of said nucleotide moieties do not comprise a blocking group at a 3′ position of a sugar of said plurality of said nucleotide moieties, optionally, comprising a 3′-0-azido group, a 3′-O-azidomethyl group, a 3′-O-alkyl hydroxylamino group, a 3′-phosphorothioate group, a 3′-O-malonyl group, a 3′-O-benzyl group, or a 3′-O-amino group or derivatives thereof. Embodiment 151. The composition of embodiment 143-150, wherein said plurality of said primed circular nucleic acid sequences comprise a concatemer of two or more repeats of the same sequence. Embodiment 152. The composition of embodiment 143-151, further comprising two or more of said polymer-nucleotide conjugate composition comprising a first polymer-nucleotide conjugate composition and a second polymer-nucleotide conjugate composition, wherein said first polymer-nucleotide conjugate composition comprises a nucleotide moiety having nucleobase type that differs from a nucleobase type of a nucleotide moiety of said second polymer-nucleotide conjugate composition. Embodiment 153. The composition of embodiment 143-152, further comprising three or more of said polymer-nucleotide conjugate composition comprising a first polymer-nucleotide conjugate composition, a second polymer-nucleotide conjugate composition, and a third polymer-nucleotide conjugate composition, wherein each of said first polymer-nucleotide conjugate composition, said second polymer-nucleotide conjugate composition, and said third polymer-nucleotide conjugate composition comprises a nucleotide moiety having a distinct nucleobase type. Embodiment 154. The composition of embodiment 143-153, further comprising four of said polymer-nucleotide conjugate composition comprising a first polymer-nucleotide conjugate composition, a second polymer-nucleotide conjugate composition, a third polymer-nucleotide conjugate composition, and a fourth polymer-nucleotide conjugate composition, wherein each of said first polymer-nucleotide conjugate composition, said second polymer-nucleotide conjugate composition, said third polymer-nucleotide conjugate composition, and said fourth polymer-nucleotide conjugate composition comprises a nucleotide moiety having a distinct nucleobase type. Embodiment 155. The composition of embodiment 143-154, wherein a primed circular nucleic acid sequences of said plurality of said primed circular nucleic acid sequences comprises one or more unique molecular identifiers (UMI). Embodiment 156. The composition of embodiment 143-155, further comprising a single-stranded oligonucleotide molecule, wherein a 5′ end and a 3′ end of said single-stranded oligonucleotide molecule is coupled to a 3′ end and a 5′ end of said primed circular nucleic acid sequences, respectively.

Embodiment 157. The composition of embodiment 143-156, wherein said primed circular nucleic acids comprises derivatives thereof. Embodiment 158. The composition of embodiment 143-157, wherein said derivatives comprise said single-stranded oligonucleotide molecule. Embodiment 159. The composition of embodiment 143-158, wherein said derivatives do not comprise said single-stranded oligonucleotide molecule. Embodiment 160. The composition of embodiment 143-159, wherein said single-stranded oligonucleotide molecule comprises between about 20-30 contiguous nucleotides. Embodiment 161. The composition of embodiment 143-160, wherein a primed circular nucleic acid sequences of said plurality of said primed circular nucleic acid sequences comprises one or more adaptors comprising an index site having a nucleic acid sequence corresponding to at least a portion of a capture nucleic acid molecule coupled to said surface. Embodiment 162. The composition of embodiment 161, wherein said index site comprises less than or equal to about 25 contiguous nucleotides. Embodiment 163. The composition of embodiment 161, wherein said index site comprises less than or equal to about 10 contiguous nucleotides. Embodiment 164. The composition of embodiment 161, wherein said index site comprises between about 5 and 25 contiguous nucleotides.

### Definitions

Unless defined otherwise, all technical and scientific terms used herein have the same meaning as commonly understood to one of ordinary skill in the art to which this disclosure belongs. It is to be understood that the foregoing general description and the following detailed description are exemplary and explanatory only and are not restrictive of any subject matter claimed.

As used herein and in the appended claims, the singular forms “a,” “and,” and “the” include plural referents unless the context clearly dictates otherwise. Also, the use of “and” means “and/or” unless stated otherwise. Similarly, “comprise,” “comprises,” “comprising” “include,” “includes,” and “including” are interchangeable and not intended to be limiting.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, +plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

A “nucleic acid molecule” is a linear polymer of two or more nucleotides joined by covalent internucleosidic linkages, or variant or functional fragments thereof. In naturally occurring examples of these, the internucleoside linkage is typically a phosphodiester bond. However, other examples may comprise other internucleoside linkages, such as phosphorothiolate linkages and may or may not comprise a phosphate group. The nucleic acid molecules include double- and single-stranded DNA, as well as double- and single-stranded RNA, DNA:RNA hybrids, peptide-nucleic acids (PNAs) and hybrids between PNAs and DNA or RNA, and also include other types of modifications. The nucleic acid molecule may be attached to one or more non-nucleotide moieties such as labels and other small molecules, large molecules such proteins, lipids, sugars, and solid or semi-solid supports, for example through either the 5′ or 3′ end.

The term “nucleotide” as used herein refers to a molecule comprising an aromatic base, a sugar, and a phosphate. A “nucleotide moiety” as referred to here can be a nucleotide or a nucleoside that is modified, such as for example, a nucleotide moiety conjugated to a polymer core or linker (e.g., in a polymer-nucleotide conjugate). Canonical or non-canonical nucleotides are consistent with use of the term. The phosphate in some instances comprises a monophosphate, diphosphate, or triphosphate, or corresponding phosphate analog. Occasionally, “nucleotide” is used informally to refer to a base in a nucleic acid molecule.

Nucleic acids may be attached to one or more non-nucleotide moieties such as labels and other small molecules, large molecules (such as proteins, lipids, sugars, e/c.), and solid or semi-solid supports, for example through covalent or non-covalent linkages with either the 5′ or 3′ end of the nucleic acid. Labels include any moiety that is detectable using any of a variety of detection methods, and thus renders the attached oligonucleotide or nucleic acid similarly detectable. Some labels emit electromagnetic radiation that is optically detectable or visible. Alternately or in combination, some labels comprise a mass tag that renders the labeled oligonucleotide or nucleic acid visible in mass spectral data, or a redox tag that renders the labeled oligonucleotide or nucleic acid detectable by amperometry or voltammetry. Some labels comprise a magnetic tag that facilitates separation and/or purification of the labeled oligonucleotide or nucleic acid. The nucleotide or polynucleotide is often not attached to a label, and the presence of the oligonucleotide or nucleic acid is directly detected.

The term “barcode” as used herein refers to a natural or synthetic nucleic acid sequence comprised by a polynucleotide allowing for unambiguous identification of the polynucleotide and other sequences comprised by the polynucleotide having said barcode sequence. The number of different barcode sequences theoretically possible can be directly dependent on the length of the barcode sequence; e.g., if a DNA barcode with randomly assembled adenine, thymidine, guanosine and cytidine nucleotides can be used, the theoretical maximal number of barcode sequences possible can be 1,048,576 for a length of ten nucleotides, and can be 1,073,741,824 for a length of fifteen nucleotides.

As used herein, the terms “DNA hybridization” and “nucleic acid hybridization” are used interchangeably, and are intended to cover any type of nucleic acid hybridization, e.g., DNA hybridization, RNA hybridization, etc., unless otherwise specified. Hybridization may occur through Watson-Crick base pairing, Hoogsteen pairing, G-loop pairing, or any mechanism for the specific and/or ordered noncovalent interaction of bases within two or more nucleic acid strands. “Hybridization” may comprise interactions between segments of a single molecule, two molecules, or more than two molecules of a nucleic acid

In some embodiments, the methods and compositions of the present disclosure comprise a label, such as a fluorescent label or a fluorophore. In some embodiments, the label is a fluorophore. Fluorescent moieties which may serve as fluorescent labels or fluorophores include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-3-{1-[6-2,5-dioxopyrrolidin-1-yloxy) oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl) ((1E,3 E)-5-((E)-1-(6-((2,5-dioxopyrrolidin-1-yl)oxy)-6-oxohexyl)-3,3-dimethyl-5-indolin ylidene)penta-1,3-dien-1-yl)-3,3-dimethyl-3H-indol-1-ium or 1-(6-((2,5-dioxopyrrolidin-1-yl)oxy) oxohexyl)-2-((1E,3E)-5-((E)-1-(6-((2,5-dioxopyrrolidin-1-yl)oxy)-6-oxohexyl)-3,3-dimethyl sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5 E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

An “organic solvent,” as used herein refers to a solvent or solvent system comprising carbon-based or carbon-containing substance capable of dissolving or dispersing other substances. An organic solvent may be miscible or immiscible with water.

A “polar solvent,” as used herein and referring to the hybridization composition described herein, is a solvent or solvent system comprising one or more molecules characterized by the presence of a permanent dipole moment, e.g., a molecule having a spatially unequal distribution of charge density. A polar solvent may be characterized by a dielectric constant of 20, 25, 30, 35, 40, 45, 50, 55, 60 or higher or by a value or a range of values incorporating any of the aforementioned values. For example, a polar solvent may have a dielectric constant of higher than 100, higher than 110, higher than 111, or higher than 115. A polar solvent as described herein may comprise a polar aprotic solvent. A polar aprotic solvent as described herein may further contain no ionizable hydrogen in the molecule. In addition, polar solvents or polar aprotic solvents may be preferably substituted in the context of the presently disclosed compositions with a strong polarizing functional groups such as nitrile, carbonyl, thiol, lactone, sulfone, sulfite, and carbonate groups so that the underlying solvent molecules have a dipole moment. Polar solvents and polar aprotic solvents can be present in both aliphatic and aromatic or cyclic form. In some embodiments, the polar solvent is acetonitrile.

The term “support” includes any solid or semisolid article on which reagents such as nucleic acids can be immobilized. Nucleic acids may be immobilized on the solid support by any method including but not limited to physical adsorption, by ionic or covalent bond formation, or combinations thereof. A solid support may include a polymeric, a glass, or a metallic material. Examples of solid supports include a membrane, a planar surface, a microtiter plate, a bead, a filter, a test strip, a slide, a cover slip, and a test tube, means any solid phase material upon which an oligomer is synthesized, attached, ligated or otherwise immobilized. A support may comprise a “resin”, “phase”, “surface,” “substrate,” “coating,” and/or “support.” A support may comprise organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Surfaces may be planar, substantially planar, or non-planar. Supports may be porous or non-porous, and may have swelling or non-swelling characteristics. A support can be shaped to comprise one or more wells, depressions or other containers, vessels, features or locations. A plurality of supports may be configured in an array at various locations. A support may be addressable (e.g., for robotic delivery of reagents), or by detection means including scanning by laser illumination and confocal or deflective light gathering. An amplification support (e.g., a bead) can be placed within or on another support (e.g., within a well of a second support).

As used herein, fluorescence is “specific” if it arises from fluorophores that are annealed or otherwise tethered to the surface, such as through a nucleic acid having a region of reverse complementarity to a corresponding segment of an oligo on the surface and annealed to said corresponding segment. This fluorescence is contrasted with fluorescence arising from fluorophores not tethered to the surface through such an annealing process, or in some cases to background florescence of the surface.

As used herein, a “liquid phase” is considered continuous if any portion of the liquid phase is in fluid contact or communication with any other portion of the liquid body. For example, a liquid phase may be considered continuous if no portion is entirely subdivided or compartmentalized or otherwise entirely physically separated from the rest of the liquid body. In some cases, a liquid phase may be flowable. In some cases, a continuous liquid phase is not within a gel or matrix. In other cases, a continuous liquid phase may be within a gel or matrix. For example, a continuous liquid phase may occupy pores, spaces or other interstices of a solid or semisolid support.

As used herein, “paired-end” information refers to genetic sequence information pertaining to both the forward and reverse strands of a double stranded nucleic acid molecule or nucleic acid segment. A paired-end read or paired-end sequencing thus refers to the determination of the sequence of both the forward and the reverse strand. This determination may be made directly and may in some embodiments be made without reference to the sequence of a known complementary strand.

### EXAMPLES

### Example 1: Circular Library Preparation in Solution

DNA is sheared into fragments and circularized. Rolling circle amplification in solution produces multiple interlinked templates. The library is sequenced. Compared to conventional sequencing methods, read intensity and efficiency is increased.

### Example 2. Performing Paired-End Sequencing

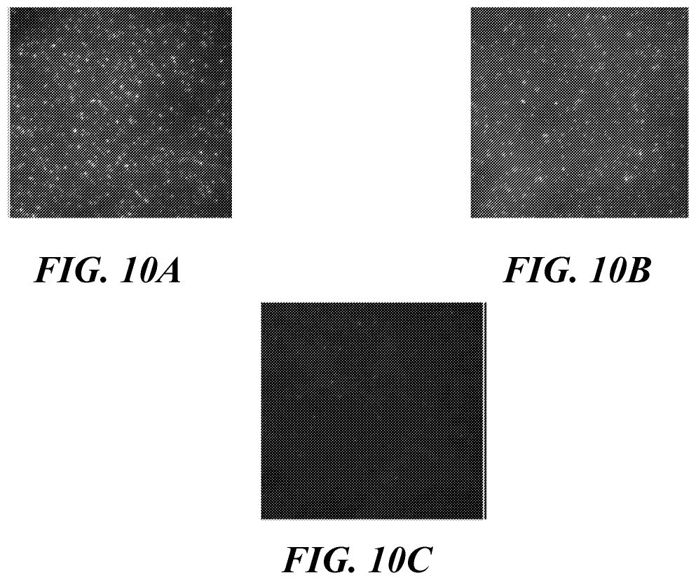

Two DNA libraries were produced using the methods described herein; a circular library, a linear library. The DNA libraries were sequenced using methods described herein. FIG. 10A depicts an example of sequencing signals generated by the method disclosed herein. FIG. 10B depicts an example of sequencing signals generated by ligation based circulation. FIG. 10C depicts an example of sequencing signals generated by uncircularized library. The circular nucleic acid library generated by methods disclosed herein demonstrates brighter signals with better signal to noise ratio compared to the library created by ligation based circulation or the uncircularized library.

### Example 3: Solid Surface Circular Library Preparation

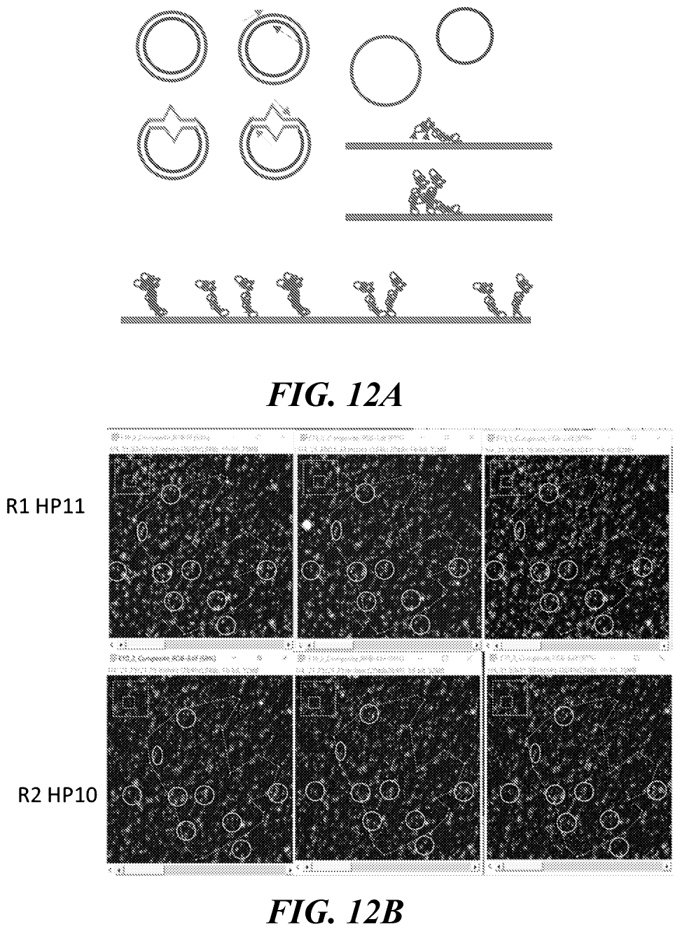

As depicted in FIG. 12A, adapters were ligated to a sheared DNA duplex. The circular DNA was denatured. The circular DNA was attached to the solid surface via adapters and amplified through rolling circle adaptation. The library was then sequenced.

FIG. 12B shows 3 consecutive rounds of sequencing data of paired-end sequencing, from both the first read (R1) and the second read (R2). As indicated by the dots, sequencing occurred throughout all 3 rounds.

### Example 4: Hairpin Loop Circular Library Preparation

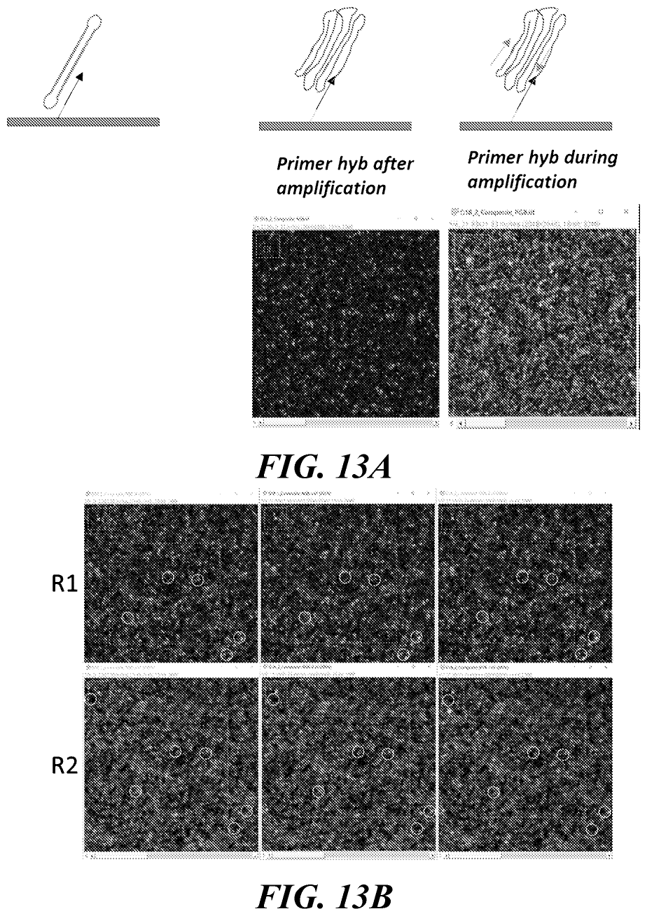

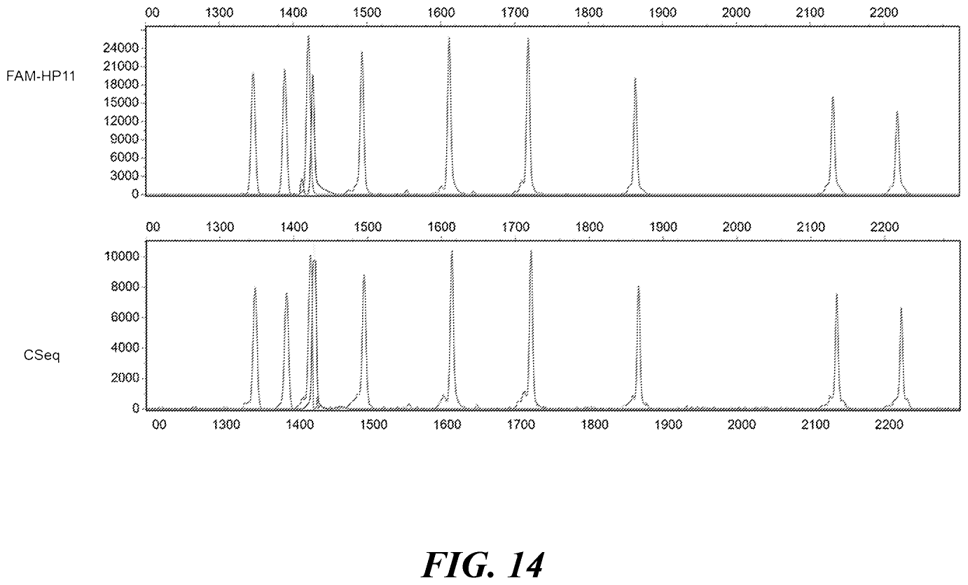

DNA was sheared into fragments. A hairpin loop adapter was used to circularize DNA. Rolling circle amplification occurred. The template was sequenced. When the sequencing primer was hybridized during amplification, there was a greater signal that when the primer was hybridized after amplification, as depicted in FIG. 13A.

FIG. 13B shows 3 consecutive rounds of sequencing data of paired-end sequencing, from both the first read (R1) and the second read (R2). As indicated by the dots, sequencing occurred throughout all 3 rounds.

### Example 5: Paired End Sequencing Strategies

PCR-free asymmetric adapters were used. A library was prepared using both strand 1 and strand 2 of the sheared DNA. The processivity of the control-seq was measured, as depicted in FIG. 14.

### Example 6. In-Solution Splint Ligation

To circularize the library, the splint oligo was used to hybridize to library outer adapters and DNA ligase sealed the nick formed by linear library and splint oligo. After ligation, non-circular DNA molecules were digested with exonucleases, followed by a SPRI beads clean-up. Final product contained only circular libraries ready for loading onto Flow Cells and Amp/Sequencing.

### Example 7. On-Surface Splint Ligation

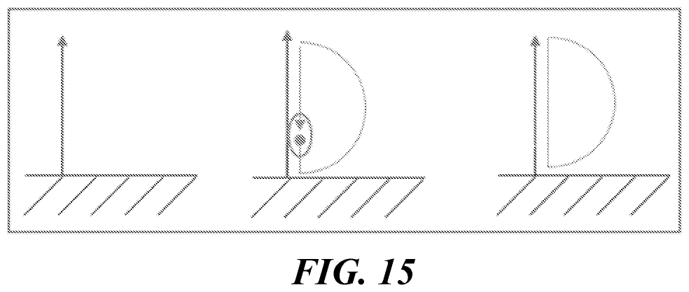

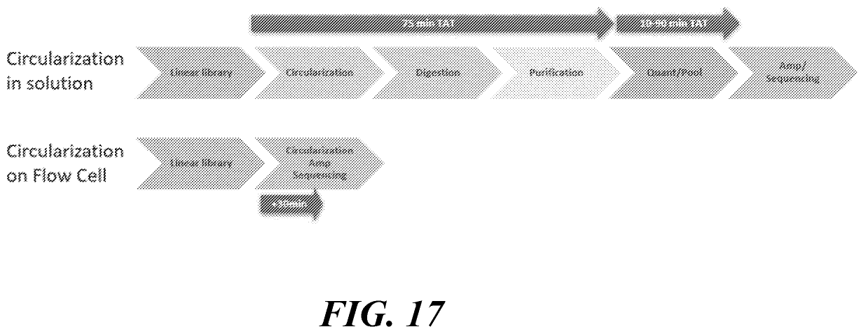

With flow cells containing splint oligos as surface primers, linear libraries were loaded directly. The splint oligos on flow cell were used to hybridize to library outer adapters and DNA ligase seals the nick formed by linear library and splint oligo. After ligation, non-circular DNA molecules and DNA ligase were washed away by universal wash buffer without the need for exonuclease digestion. Circularized libraries on flow cells are ready for amplification and sequencing. A non-limiting schematic of on-surface splint ligation is provided in FIG. 15. FIG. 17 shows a non-limiting schematic of in-solution splint ligation compared with on-surface splint ligation, and shows that on-surface splint ligation can reduce the reaction time by at least 75 minutes because it obviates a need for digestion, purification, and quantification/pooling.

### Example 8. Comparison of On-Surface Splint Ligation and In-Solution Splint Ligation

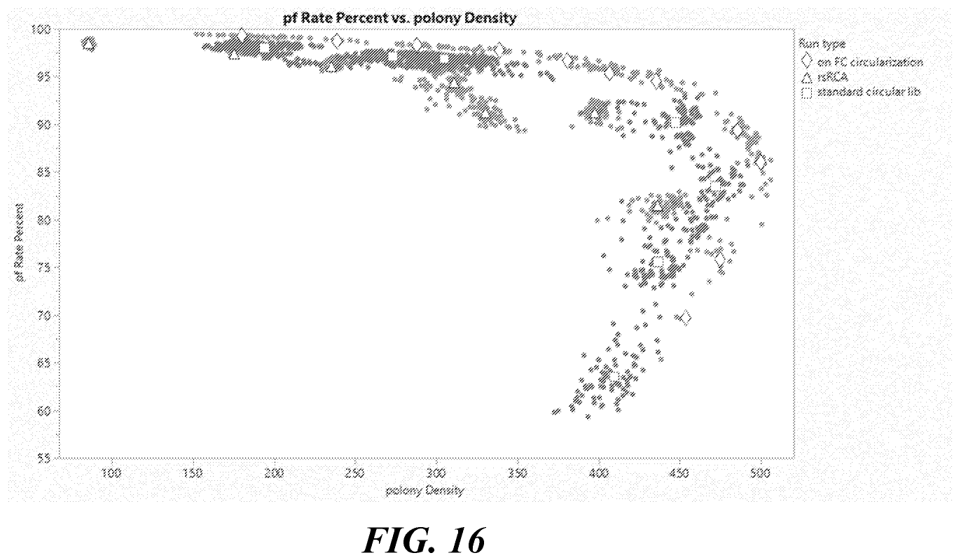

The same library was split into two portion A and B. Portion A was processed using circularization in solution method (circularization with splint oligos, ligation, digestion, clean-up), then loaded onto flow cells for amplification and sequencing. Portion B was loaded as linear format onto flow cells with splint oligos as surface primers, then circularized with ligase and subjected to amplification and sequencing. FIG. 16 shows the polymerase colony (“polony”) density and size produced from various library input concentrations. On-surface circularization yielded more consistent polony densities than other methods under the conditions tested.

### Example 9. Sequencing Circularized Nucleic Acid Molecules

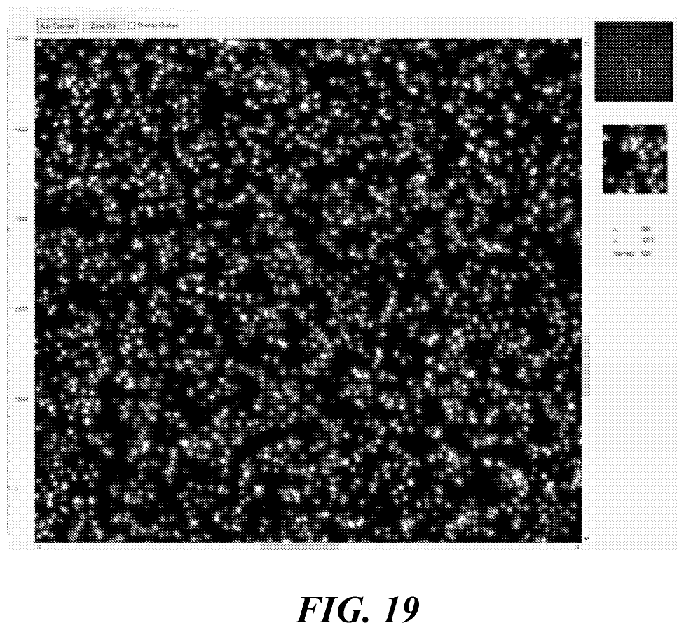

library of circularized template nucleic acid molecules was prepared as described in the previous examples and coupled to an interior surface of a flow cell. A primer sequence was hybridized to the circularized template nucleic acid molecules and amplicons thereof. The primed templates were blocked with terminator chain nucleotides described herein to prevent further incorporation. Polymerase and four types fluorescently-labeled polymer-nucleotide conjugates (e.g., with nucleotide moieties have nucleobases A, T, C, G) were flowed into the flow a binding complexes formed between the polymer-nucleotide conjugate and the nucleotide of the primed template are imaged by fluorescence. FIG. 19 shows a fluorescent microscopy image of the surface during a sequencing reaction.

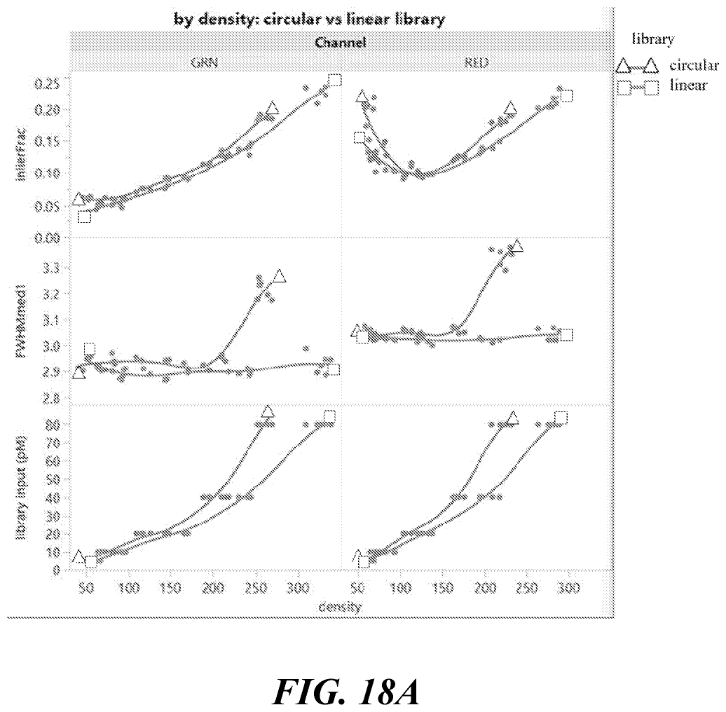

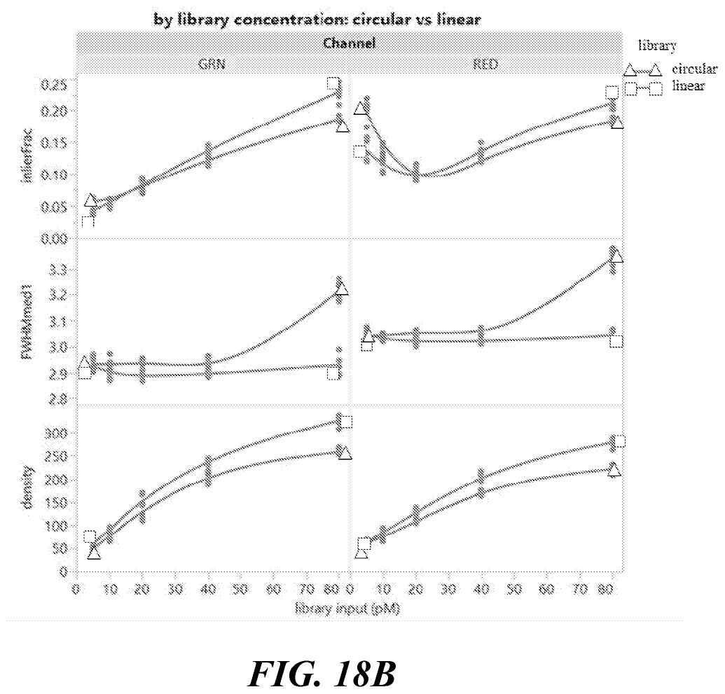

FIG. 18A shows imaging analysis of polonies on a surface using red or green detection channels, circular library is shown in blue, and linear library is shown in red. On the X-axis is polony density in units of thousand (K)/millimeter (mm)²; on the Y-axis, from top to bottom: Inlier fraction: a metric used to describe how much overlapping of polonies occurs on a support. A high inlier fraction is not desirable; FWHM=“full width half max,” a measurement of the full width or approximate diameter of a polony at the ring of intensity corresponding to one half of the maximal intensity measured for that polony (an indicator of the width of an image of an immobilized polony); and Library input in pmol. FIG. 18B shows a comparison of library concentration in circular vs. linear libraries. Imaging analysis of input library concentration on a surface using red or green detection channels is plotted (Circular library=triangle, linear library=square). On the X-axis is Library input in pmol; on the Y-axis, from top to bottom, is inlier fraction, as defined above; FWHM, as defined above; and density on the support in K/mm².

### Example 10. On-Surface Ligation—Sequencing Results

Study 1

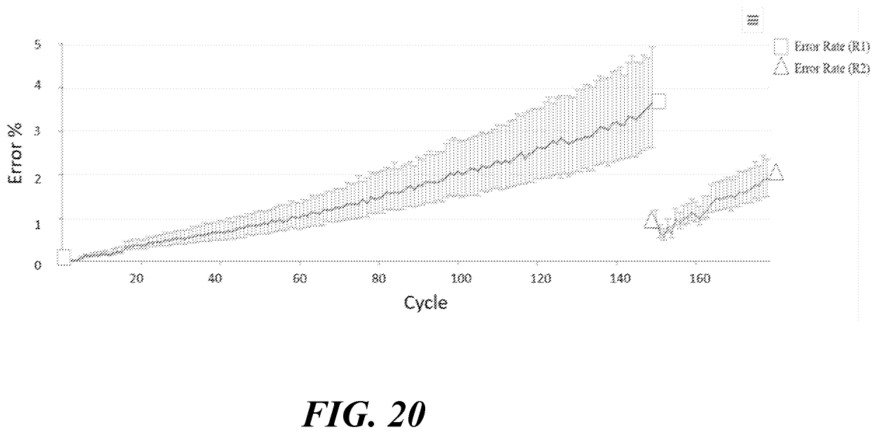

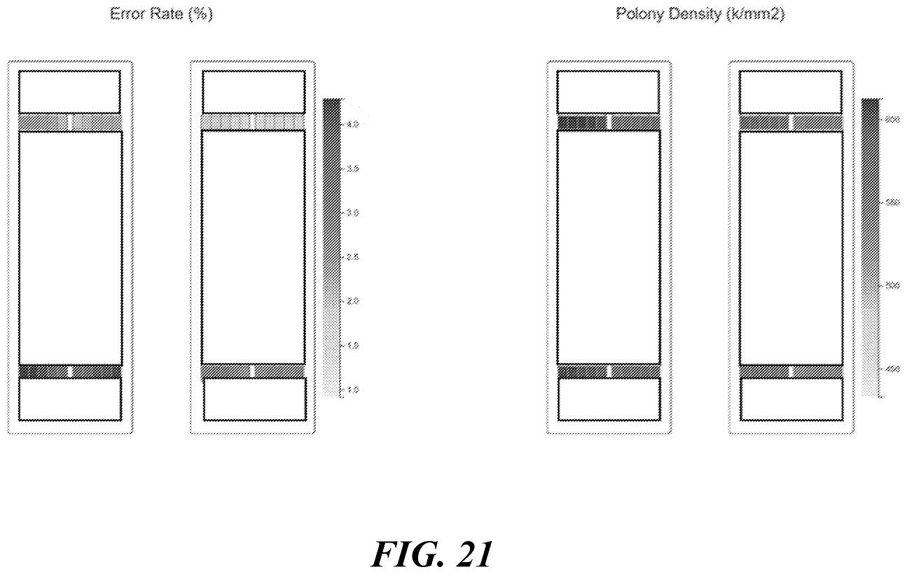

A linear library was mixed with DNA ligase and loaded directly onto flow cells with splint oligos as surface primers. After ligation, the circular library was amplified and sequenced using avidity chemistry for 150 cycles in read 1 and 30 cycles in read 2. FIG. 20 shows the average error rate line in read 1 on the left and line in read 2 on the right) with variation among tiles (grey lines). Plots in FIG. 21 are heat maps of error rate (left) and polony density (right).

Study 2

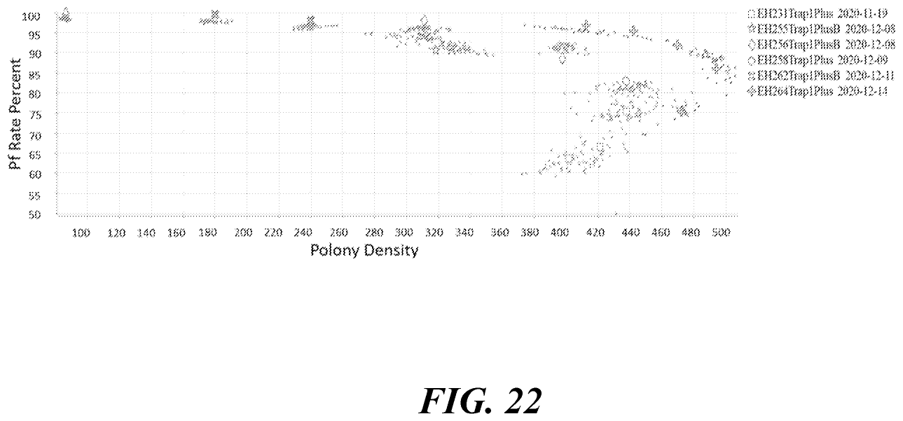

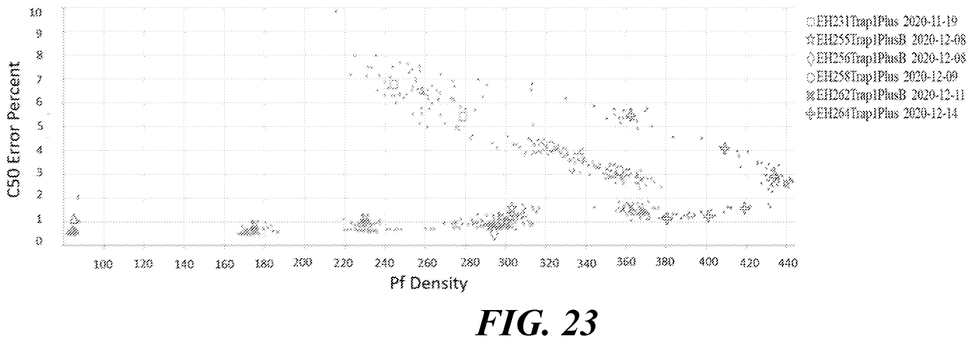

A linear library was mixed with DNA ligase and loaded directly onto flow cells with splint oligos as surface primers. After ligation, the circular library was amplified and sequenced with avidity chemistry for 91 cycles. FIG. 22 shows pass filter rate by tile across flow cells. Boxed data points were high PF tiles from on-FC circularization method comparing circularization in solution (non-boxed plots). FIG. 23 shows error rate at cycle 50 of the same sequencing runs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of nucleic acid sequencing, said method comprising:
(a) bringing a nucleic acid sequence or derivative thereof into contact with a surface under conditions sufficient to couple said nucleic acid sequence or derivative thereof to said surface;
(b) enzymatically circularizing said nucleic acid sequence or derivative thereof to produce a circular nucleic acid sequence;
(c) contacting said circular nucleic acid sequence or derivative thereof with a primer sequence complementary thereto, thereby producing a primed nucleic acid sequence; and
(d) performing a nucleotide binding reaction with said primed nucleic acid sequence or derivative thereof to identify a nucleotide of said primed nucleic acid sequence or derivative thereof, which nucleotide binding reaction is performed in absence of incorporation of a nucleotide into said primed nucleic acid sequence or derivative thereof.

2. The method of claim 1, wherein said enzymatically circularizing said nucleic acid sequence comprises performing splint ligation.

3. The method of claim 1, wherein (a) comprises bringing a fluid comprising said nucleic acid sequence at a concentration of less than or equal to about 1 nanomolar (nM) into contact with said surface.

4. The method of claim 1, wherein (a) comprises bringing a fluid comprising said nucleic acid sequence at a concentration of less than or equal to about 100 picomolar (pM) into contact with said surface.

5. The method of claim 1, wherein (a) comprises bringing a fluid comprising said nucleic acid sequence at a concentration comprising greater than or equal to about 80 picomolar (pM) into contact with said surface.

6. The method of claim 1, wherein (a) comprises bringing a fluid comprising said nucleic acid sequence or derivative thereof at a concentration comprising between about 20 pM and about 1 nM.

7. The method of claim 1, wherein said primed nucleic acid sequence or derivative thereof is coupled to said surface at a surface density of greater than or equal to about 4,000 primed nucleic acid sequences per micrometer (μm)².

8. The method of claim 1 or 7, wherein said primed nucleic acid sequence or derivative thereof is coupled to said surface at a surface density of less than or equal to about 15,000 primed nucleic acid sequences per μm².

9. The method of claim 1, wherein a plurality of colonies comprising said primed nucleic acid sequence or derivative thereof is present at said surface at a density of greater than or equal to about 300 thousand (K)/mm².

10. The method of claim 9, wherein said colony density comprises less than or equal to about 500 K/mm².

11. The method of claim 1, wherein said primed nucleic acid sequence or derivative thereof comprises one or more adaptors comprising an index site having a sequence complementary to at least a portion of a capture nucleic acid molecule coupled to said surface.

12. The method of claim 11, wherein said index site comprises less than or equal to about 25 contiguous nucleotides.

13. The method of claim 11, wherein said index site comprises less than or equal to about 10 contiguous nucleotides.

14. The method of claim 11, wherein said index site comprises between about 5 and 25 contiguous nucleotides.

15. The method of claim 1, wherein said surface comprises a hydrophilic polymer layer coupled thereto.

16. The method of claim 1, wherein said primed nucleic acid sequence or derivative thereof comprises a concatemer of two or more repeats of an identical sequence.

17. The method of claim 1, further comprising amplifying said circular nucleic acid sequence or derivative thereof using rolling circle amplification (RCA) prior to (c).

18. The method of claim 1, further comprising:
(e) performing a primer extension reaction on said primed nucleic acid sequence or derivative thereof; and
(f) repeating (a) to (e) for each successive nucleotide to identify a sequence of said primed nucleic acid sequence or derivative thereof.

19. The method of claim 18, wherein (a)-(f) are performed in less than or equal to about 120 minutes.

20. The method of claim 18, wherein (d)-(e) are performed in less than or equal to about 15 minutes.

21. The method of claim 18, wherein (f) is performed in less than or equal to about 15 minutes.

22. The method of claim 1, wherein performing said nucleotide binding reaction in (d) comprises:
(i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and
(ii) detecting said stable multivalent binding complex to determine identity of said nucleotide of said primed nucleic acid sequence or derivative thereof.

23. The method of claim 22, wherein said one or more polymer-nucleotide conjugates comprises a polymer core and a detectable label coupled thereto.

24. The method of claim 22, wherein said one or more polymer-nucleotide conjugates comprises two or more types of said one or more polymer-nucleotide conjugates.

25. The method of claim 22, wherein said one or more polymer-nucleotide conjugates comprises three or more types of said one or more polymer-nucleotide conjugates.

26. The method of claim 22, wherein said one or more polymer-nucleotide conjugates comprises four types of said one or more polymer-nucleotide conjugates.

27. The method of claim 22, wherein said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type.

28. The method of claim 22, wherein said one or more polymer-nucleotide conjugates comprises a plurality of types of polymer-nucleotide conjugates, and wherein each of said plurality of types of said polymer-nucleotide conjugates comprises a distinct detectable label.

29. The method of claim 1, wherein said enzymatically circularizing said nucleic acid sequence or derivative thereof in (b) comprises: (i) hybridizing a 5′ end of a single-stranded nucleic acid molecule to a 3′ end of said nucleic acid sequence or derivative thereof and hybridizing a 3′ end of said single-stranded nucleic acid molecule to a 5′ end of said nucleic acid sequence or derivative thereof, or (ii) hybridizing a 3′ end of a single-stranded nucleic acid molecule to a 5′ end of said nucleic acid sequence or derivative thereof and hybridizing a 5′ end of said single-stranded nucleic acid molecule to a 3′ end of said nucleic acid sequence or derivative thereof.

30. The method of claim 29, wherein said single-stranded nucleic acid molecule comprises between about 20-30 contiguous nucleotides.

31. The method of claim 1, wherein said nucleic acid sequence or derivative thereof comprises one or more unique molecular identifiers (UMI) at a 5′ end or a 3′ end thereof.

32. The method of claim 1, further comprising: adding one or more adaptors to a 5′ end or a 3′ end of said nucleic acid sequence or derivative thereof comprising an index site having a nucleic acid sequence corresponding to at least a portion of a capture nucleic acid molecule coupled to said surface.

33. The method of claim 32, wherein said index site comprises less than or equal to about 25 contiguous nucleotides.

34. The method of claim 32, wherein said index site comprises less than or equal to about 10 contiguous nucleotides.

35. The method of claim 32, wherein said index site comprises between about 5 and 25 contiguous nucleotides.

36. The method of claim 1, wherein said enzymatically circularizing said nucleic acid sequence or derivative thereof comprises ligating a 5′ end and a 3′ end of said nucleic acid sequence or derivative thereof together under conditions sufficient to produce said circular nucleic acid sequence or derivative thereof.

37. The method of claim 1, further comprising performing (a) to (d) for a plurality of said nucleic acid sequence or derivative thereof.

38. The method of claim 1, further comprising incorporating a nucleotide into said primed nucleic acid sequence.

39. A method of nucleic acid sequencing, said method comprising:
(a) circularizing a nucleic acid sequence to provide a circular nucleic acid sequence coupled to a surface;
(b) contacting said circular nucleic acid sequence or derivative thereof with a primer sequence complementary thereto, thereby producing a primed nucleic acid sequence; and
(c) performing a nucleotide binding reaction with said primed nucleic acid sequence or derivative thereof to identify a nucleotide of said primed nucleic acid sequence or derivative thereof, which nucleotide binding reaction is performed in absence of incorporation of a nucleotide into said primed nucleic acid sequence or derivative thereof.

40. The method of claim 39, wherein said circularizing said nucleic acid sequence thereof comprises performing splint ligation.

41. The method of claim 1, wherein said circular nucleic acid sequence is coupled to said surface at a surface density of greater than or equal to about 4,000 primed nucleic acid sequences per micrometer (μm)².

42. The method of claim 1 or 7, wherein said circular nucleic acid sequence is coupled to said surface at a surface density of less than or equal to about 15,000 primed nucleic acid sequences per μm².

43. The method of claim 1, wherein a plurality of colonies comprising said circular nucleic acid sequence or derivative thereof is present at said surface at a density of greater than or equal to about 300 K/mm².

44. The method of claim 43, wherein said colony density comprises less than or equal to about 500 K/mm².

45. The method of claim 39, wherein said circular nucleic acid sequence or derivative thereof comprises one or more adaptors comprising an index site having a sequence complementary to at least a portion of a capture nucleic acid molecule coupled to said surface.

46. The method of claim 45, wherein said index site comprises less than or equal to about 25 contiguous nucleotides.

47. The method of claim 45, wherein said index site comprises less than or equal to about 10 contiguous nucleotides.

48. The method of claim 45, wherein said index site comprises between about 5 and 25 contiguous nucleotides.

49. The method of claim 39, wherein said surface comprises a hydrophilic polymer layer coupled thereto.

50. The method of claim 39, wherein said circular nucleic acid sequence or derivative thereof comprises a concatemer of two or more repeats of an identical sequence.

51. The method of claim 39, further comprising amplifying said circular nucleic acid sequence or derivative thereof using rolling circle amplification (RCA) prior to (c).

52. The method of claim 51, wherein said rolling circle amplification is performed in at least about 10 minutes to at least about 90 minutes.

53. The method of claim 39, further comprising:
(e) performing a primer extension reaction on said primed nucleic acid sequence or derivative thereof; and
(f) repeating (a) to (e) for each successive nucleotide to identify a sequence of said primed nucleic acid sequence or derivative thereof.

54. The method of claim 53, wherein (a)-(f) are performed in less than or equal to about 120 minutes.

55. The method of claim 53, wherein (d)-(e) are performed in less than or equal to about 15 minutes.

56. The method of claim 53, wherein (f) is performed in less than or equal to about 15 minutes.

57. The method of claim 39, wherein performing said nucleotide binding reaction in (d) comprises:
(i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and
(ii) detecting said stable multivalent binding complex to determine said identity of said nucleotide of said primed nucleic acid sequence or derivative thereof.

58. The method of claim 57, wherein said one or more polymer-nucleotide conjugates comprises a polymer core and a detectable label coupled thereto.

59. The method of claim 57, wherein said one or more polymer-nucleotide conjugates comprises two or more types of said one or more polymer-nucleotide conjugates.

60. The method of claim 57, wherein said one or more polymer-nucleotide conjugates comprises three or more types of said one or more polymer-nucleotide conjugates.

61. The method of claim 57 wherein said one or more polymer-nucleotide conjugates comprises four types of said one or more polymer-nucleotide conjugates.

62. The method of claim 57, wherein said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type.

63. The method of claim 57, wherein said one or more polymer-nucleotide conjugates comprises a plurality of types of polymer-nucleotide conjugates, and wherein each of said plurality of types of said polymer-nucleotide conjugates comprises a distinct detectable label.

64. The method of claim 39, wherein said circularizing said nucleic acid sequence or derivative thereof in (b) comprises: (i) hybridizing a 5′ end of a single-stranded nucleic acid molecule to a 3′ end of said nucleic acid sequence or derivative thereof and hybridizing a 3′ end of said single-stranded nucleic acid molecule to a 5′ end of said nucleic acid sequence or derivative thereof, or (ii) hybridizing a 3′ end of a single-stranded nucleic acid molecule to a 5′ end of said nucleic acid sequence or derivative thereof and hybridizing a 5′ end of said single-stranded nucleic acid molecule to a 3′ end of said nucleic acid sequence or derivative thereof.

65. The method of claim 64, wherein said single-stranded nucleic acid molecule comprises between about 20-30 contiguous nucleotides.

66. The method of claim 39, wherein said nucleic acid sequence or derivative thereof comprises one or more unique molecular identifiers (UMI) at a 5′ end or a 3′ end thereof.

67. The method of claim 39, further comprising: adding one or more adaptors to a 5′ end or a 3′ end of said nucleic acid sequence or a derivative thereof comprising an index site having a nucleic acid sequence corresponding to at least a portion of a capture nucleic acid molecule coupled to said surface.

68. The method of claim 67, wherein said index site comprises less than or equal to about 25 contiguous nucleotides.

69. The method of claim 67, wherein said index site comprises less than or equal to about 10 contiguous nucleotides.

70. The method of claim 67, wherein said index site comprises between about 5 and 25 contiguous nucleotides.

71. The method of claim 1, wherein said enzymatically circularizing said nucleic acid sequence or derivative thereof comprises ligating a 5′ end and a 3′ end of said nucleic acid sequence or derivative thereof together under conditions sufficient to produce said circular nucleic acid sequence or derivative thereof.

72. The method of claim 39, further comprising performing (a) to (d) for a plurality of said nucleic acid sequence or derivative thereof.

73. The method of claim 39, further comprising incorporating a nucleotide into said primed nucleic acid sequence.

74. A system for nucleic acid sequencing, said system comprising:
a surface; and
one or more computer processors individually or collectively programmed to implement a method comprising:
(a) bringing a nucleic acid sequence into contact with said surface under conditions sufficient to couple said nucleic acid sequence or derivative thereof to said surface;
(b) enzymatically circularizing said nucleic acid sequence or a derivative thereof to produce a circular nucleic acid sequence;
(c) contacting said circular nucleic acid sequence or derivative thereof with a primer sequence complementary thereto, thereby producing a primed nucleic acid sequence; and
(d) performing a nucleotide binding reaction with said primed nucleic acid sequence or a derivative thereof to identify a nucleotide of said primed nucleic acid sequence or derivative thereof.

75. The system of claim 74, further comprising: a first fluid comprising a synthetic ligating enzyme or enzymatically-active fragment thereof, and a synthetic splint nucleic acid molecule.

76. The system of claim 74, further comprising: a second fluid comprising one or more nucleotide moieties and a polymerizing enzyme.

77. The system of claim 74, wherein said surface comprises a hydrophilic polymer layer coupled thereto.

78. The system of claim 74, further comprising an imaging module comprising one or more light sources, one or more optical components, and one or more image sensors operably connected to said surface for detecting said binding complex.

79. The system of claim 74, further comprising a fluidics module configured to bring said nucleic acid sequence or derivative thereof into contact with said surface in (b).

80. The system of claim 74, wherein said method further comprises:
(e) performing a primer extension reaction on said primed nucleic acid sequence or derivative thereof; and
(f) repeating (a) to (e) for each successive nucleotide to identify a sequence of said primed nucleic acid sequence or derivative thereof.

81. The system of claim 80, wherein said method is performed in less than or equal to about 30 minutes.

82. The system of claim 74, wherein said method further comprises:
amplifying said circular nucleic acid sequence or a derivative thereof using rolling circle amplification (RCA) prior to (c).

83. The method of claim 82, wherein said rolling circle amplification is performed in at least about 10 minutes to at least about 90 minutes.

84. The system of claim 74, wherein said surface comprises an interior surface of a flow cell.

85. The system of claim 74, wherein performing said nucleotide binding reaction in (d) comprises:
(i) bringing said primed nucleic acid sequence or derivative thereof into contact with one or more polymer-nucleotide conjugates under conditions sufficient to form a stable multivalent binding complex between a nucleotide moiety of said one or more polymer-nucleotide conjugates and a nucleotide of said primed nucleic acid sequence or derivative thereof; and
(ii) detecting said stable multivalent binding complex to determine said identity of said nucleotide of said primed nucleic acid sequence or derivative thereof.

86. The system of claim 85, further comprising said one or more polymer-nucleotide conjugates.

87. The system of claim 85, further comprising two or more types of said one or more polymer-nucleotide conjugates.

88. The system of claim 85, further comprising three or more types of said one or more polymer-nucleotide conjugates.

89. The system of claim 85, further comprising four types of said one or more polymer-nucleotide conjugates.

90. The system of claim 85, wherein said one or more polymer-nucleotide conjugates comprises a plurality of types of polymer-nucleotide conjugates, and wherein each of said plurality of types of said polymer-nucleotide conjugates comprises a nucleotide moiety with a distinct nucleobase type.

91. The system of claim 85, wherein said one or more polymer-nucleotide conjugates comprises a plurality of types of polymer-nucleotide conjugates, and wherein each of said plurality of types of said polymer-nucleotide conjugates comprises a distinct detectable label.

92. The system of claim 85, wherein said polymer-nucleotide composition comprises a detectable label.

93. The system of claim 92, wherein said detectable label comprises a fluorescent label.

94. The system of claim 85, further comprising said nucleic acid sequence or derivative thereof, wherein said nucleic acid sequence or derivative thereof comprises one or more unique molecular identifiers (UMI) at a 5′ end or a 3′ end thereof.

95. The system of claim 85, further comprising said nucleic acid sequence or derivative thereof, wherein said nucleic acid sequence or derivative thereof comprises one or more adaptors comprising an index site having a nucleic acid sequence corresponding to at least a portion of a capture nucleic acid molecule coupled to said surface.

96. The system of claim 95, wherein said index site comprises less than or equal to about 25 contiguous nucleotides.

97. The system of claim 95, wherein said index site comprises less than or equal to about 10 contiguous nucleotides.

98. The system of claim 95, wherein said index site comprises between about 5 and 25 contiguous nucleotides.
